(19) United States
(12) Patent Application Publication
(10) Pub. No.: US 2015/0247865 A1
(43) Pub. Date: Sep. 3, 2015

(54) Polypeptide Markers for the Diagnosis and Evaluation of Vascular Diseases

(71) Applicant: Mosaiques Diagnostics And Therapeutics AG, Hannover (DE)

(72) Inventor: Harald Mischak, Sehnde (DE)

(21) Appl. No.: 14/712,423

(22) Filed: May 14, 2015

Related U.S. Application Data
(63) Continuation of application No. 12/085,633, filed on Jan. 9, 2009, now abandoned.

(30) Foreign Application Priority Data
Nov. 30, 2005 (DE) 102005057382.7
Sep. 19, 2006 (EP) 06120879.9

(51) Int. Cl. G01N 33/68; G01N 27/26; G01N 33/493

(52) U.S. Cl. CPC G01N 33/6848; G01N 33/493; G01N 27/26; G01N 2800/32

(57) ABSTRACT

A process for diagnosing vascular diseases (VD), comprising the step of determining the presence or absence of at least one polypeptide marker in a sample, wherein said polypeptide marker is selected from markers 1 to 526, which are characterized by values for the molecular masses and migration times (CE times).

### CROSS-REFERENCED APPLICATION

This application is a continuation of and claims priority to U.S. patent application Ser. No. 12/085,633, filed Jan. 9, 2009, which claims priority to PCT/EP2006/069096, filed Nov. 30, 2006, which, in turn, claims priority to European application number EP 06120879.9, filed Sep. 19, 2006 and German application number DE 102005057382.7, filed Nov. 30, 2005, the contents of which are all incorporated by reference herein in their entireties.

### BACKGROUND

1. Field of the Disclosure

The present disclosure relates to the use of the presence or absence of one or more peptide markers in a sample from a subject for the diagnosis and evaluation of severity of vascular diseases (VD) and to a method for the diagnosis and evaluation of such vascular disease, wherein the presence or absence of the peptide marker or markers is indicative of the severity of a VD.

2. Discussion of the Background Art

Vascular diseases are diseases affecting the vessels of an organism and consequently organs such as the heart, brain, kidney etc. They include, for example, arteriosclerosis, disturbed circulation, hypertension and cardiac dysrhythmia.

### Blood Vessels:

Arteriosclerosis refers to the hardening of arteries by vascular deposits. Deposits of cholesterol crystals lead to the formation of inflammatory foci (atheromas) in which blood components, lipids, metabolic slags and lime salts tend to settle. So-called plaques are formed, which are two-dimensional scleroses, whereby the vascular wall becomes more rigid and narrower. The artery loses its elasticity and has difficulty in performing its task, i.e., the transport of blood from the heart into the individual regions of the body. Secondary diseases include, for example, angina pectoris, myocardial infarction, circulatory collapse and stroke. Disturbed circulation mostly affects the lower portion of the body, from the ventral aorta to the foot arteries, and leads to a reduction of blood flow and oxygen supply to the muscular tissue, which gradually becomes necrotic. In the last stage, ulcers form and occlude the vessels to such an extent that amputation becomes unavoidable. Hypertension has no definite cause; thus, the intake of medicaments or the excessive secretion of adrenal hormones can cause the blood pressure to surge. High blood pressures are also found in permanent stress, which results in angiospasms. Hypertension damages the vascular walls, so that there is a risk of tearing or obstruction. If the regularity of the heart beat is disturbed, the condition is referred to as cardiac dysrhythmia. The heart beat may be either too fast (tachycardia), too slow (bradycardia) or irregular (arrhythmia). Vascular diseases can be avoided by prevention, because they are also caused by an unhealthy and unnatural conduct of life. By a radical reversion of the way of living, arteriosclerosis in an early stage can be stalled, e.g., by reducing the blood pressure and blood lipid levels. The progress of vascular diseases can additionally be slowed down by medicamentous therapies (e.g., acetylsalicylic acid, beta receptor blockers, ACE inhibitors etc.). However, it is to be noted that damaged vessels are irreparable, and the process in an advanced stage is irreversible. Therefore, early detection of vascular diseases is particularly important.

### Heart:

In a coronary heart disease, the diagnosis of VD is effected at first indirectly by the evaluation of risk factors and by non-invasive examinations, such as measurement of blood pressure, resting and exercise electrocardiograms, and blood pictures for determining the lipid state (LDL cholesterol, HDL cholesterol, triglycerides), fasting blood glucose level and, if necessary, HbA1c. If such examinations yield the presence of high-risk characteristics, i.e., severe vascular events (death, myocardial infarction) are to be expected in the near future, a more exact diagnosis is made by means of invasive diagnostics, e.g., in the form of a catheter examination or coronary angiography. Thus, the heart and coronary vessels and other vessels are examined by means of a catheter or with an X-ray method. X-ray contrast media are used for a better visualization of the heart and vessels on the X-ray image. Indications of coronary angiography include a low or medium preliminary test probability while non-invasive diagnostics failed to provide reliable results, patients in whom non-invasive testing is not possible due to handicaps or diseases, and patients for whom exclusion with certainty of a suspected coronary heart disease is indispensable for work-related reasons (e.g., pilots, fire fighters). However, coronary angiography can be performed only if various complications, such as hyperthyroidism or allergy to contrast media, are excluded, in addition to the above mentioned preliminary examinations. In addition, since the contrast medium is secreted through the kidney, a sufficient renal function must be ensured, or for dialysis-dependent subjects, a dialysis must be performed always subsequent to the examination. Thus, it becomes clear that there is a need for a non-invasive possibility of an early and reliable diagnosis of vascular diseases.

### Kidney:

Vascular diseases of the kidney include:
renal artery stenosis
renal artery thrombosis
renal artery embolism
renal vein thrombosis

A renal artery stenosis is a one-sided or double-sided constriction of the arteria renalis or its main branches. It may be the cause of arterial hypertension, which is then referred to as renovascular hypertension.

Its cause is arteriosclerosis (predominantly in an advanced age) in about 70% of the cases, and fibromuscular dysplasia (an abnormality of connective tissue) in about 20% of the cases. Rarely, aneurysms of the aorta or renal artery, vasculitides, mechanical compression from tumors or cysts, embolisms or thromboses are causally involved.

The constriction of the renal artery leads to a reduced blood flow through the affected kidney. In order to compensate for the presumed (local!) reduction in blood pressure, the kidney enhances the production of renin, which leads to an increase of blood volume and an increase of blood pressure of the whole organism through the angiotensin-aldosterone mechanism and thus in arterial hypertension. Therefore, renal artery stenosis is mostly discovered when a hypertension is worked up, but only about 1-2% of all hypertensions are caused thereby.

In terms of therapy, there are different possibilities:
PTA (percutaneous transluminal catheter angioplasty): the dilatation of the constriction by means of an inserted balloon catheter (balloon dilatation);
stent: insertion of a wire mesh (stent) that is to keep the vessel open;
surgical elimination of the stenosis.

A frequent cause of a renal artery thrombosis is embolisms derived from the heart, for example during atrial fibrillation, which are accompanied by symptoms such as flank pain, proteinuria, very high LDH. Flank pain is also observed in renal vein thrombosis, but additionally proteinuria and, in some cases, hematuria or a nephrotic syndrome is observed.

### Brain:

Constricted vessels in the brain region result in a reduced oxygen supply, and when an artery is occluded (e.g., by an acute clot due to the changes from arterial sclerosis), a stroke occurs with loss of perception, paralyses, disturbed speech etc. In brain arteries, such as in the large arteries, arterial sclerosis may in rare cases lead to aneurysms of the vascular walls, and together with risk factors such as hypertension, the vascular wall may tear and result in a life-threatening inner bleeding. Surprisingly, it has now been found that particular peptide markers in a urine sample from a subject can be used for the diagnosis of VD and thus to decide whether or not a medication therapy is necessary.

### SUMMARY OF THE DISCLOSURE

Thus, the present disclosure relates to the use of the presence or absence of at least one peptide marker, ideally several polypeptide markers, in a urine sample from a subject for the diagnosis of vascular diseases, wherein said polypeptide marker or markers are selected from the polypeptide markers No. 1 to No. 526, which are characterized by the molecular masses and migration times as stated in Table 1.

TABLE 1
Polypeptide markers for the diagnosis of
vascular diseases and their molecular masses
and migration times (CE time in minutes):

| No. | Mass | CE time |
|---|---|---|
| 1 | 1166.61 | 23.88 |
| 2 | 2431.50 | 24.10 |
| 3 | 1922.93 | 31.99 |
| 4 | 2509.16 | 25.76 |
| 5 | 3194.22 | 30.34 |
| 6 | 1705.80 | 40.47 |
| 7 | 1962.95 | 31.77 |
| 8 | 3822.12 | 24.72 |
| 9 | 2212.32 | 24.94 |
| 10 | 3015.78 | 35.86 |
| 11 | 1784.95 | 20.94 |
| 12 | 1902.92 | 31.87 |
| 13 | 2329.15 | 27.17 |
| 14 | 2154.05 | 21.78 |
| 15 | 2166.03 | 27.89 |
| 16 | 2258.27 | 21.99 |
| 17 | 2573.84 | 20.49 |
| 18 | 1270.75 | 37.92 |
| 19 | 1611.84 | 40.12 |
| 20 | 1791.87 | 41.04 |
| 21 | 2030.00 | 25.23 |
| 22 | 1290.40 | 30.87 |
| 23 | 1441.74 | 39.13 |
| 24 | 2924.25 | 24.05 |
| 25 | 816.41 | 21.10 |
| 26 | 963.52 | 21.71 |
| 27 | 1503.74 | 29.63 |
| 28 | 2849.59 | 23.02 |
| 29 | 3133.20 | 31.20 |
| 30 | 1283.62 | 27.30 |
| 31 | 1495.75 | 23.31 |
| 32 | 1513.70 | 29.29 |
| 33 | 1612.83 | 23.36 |
| 34 | 2319.19 | 33.80 |
| 35 | 2436.23 | 22.87 |
| 36 | 2557.42 | 28.22 |
| 37 | 2626.85 | 28.00 |
| 38 | 2933.46 | 27.68 |
| 39 | 2994.09 | 29.50 |
| 40 | 4101.34 | 28.51 |
| 41 | 935.49 | 23.69 |
| 42 | 1521.75 | 30.42 |
| 43 | 1669.79 | 21.48 |
| 44 | 2758.37 | 28.94 |
| 45 | 3546.94 | 26.22 |
| 46 | 3609.63 | 20.22 |
| 47 | 3697.49 | 23.71 |
| 48 | 4278.73 | 23.34 |
| 49 | 4421.04 | 20.73 |
| 50 | 4805.67 | 26.49 |
| 51 | 11058.18 | 21.88 |
| 52 | 1352.61 | 29.86 |
| 53 | 2802.85 | 36.35 |
| 54 | 4890.88 | 26.48 |
| 55 | 5212.06 | 26.98 |
| 56 | 945.45 | 25.80 |
| 57 | 1065.55 | 25.50 |
| 58 | 1137.58 | 26.41 |
| 59 | 1542.77 | 23.91 |
| 60 | 1693.83 | 23.47 |
| 61 | 3361.42 | 24.26 |
| 62 | 3617.74 | 26.97 |
| 63 | 3737.69 | 37.15 |
| 64 | 980.54 | 22.44 |
| 65 | 1221.63 | 26.82 |
| 66 | 2952.27 | 25.14 |
| 67 | 3696.88 | 26.94 |
| 68 | 5574.45 | 23.24 |
| 69 | 1182.59 | 28.34 |
| 70 | 1963.96 | 31.76 |
| 71 | 882.54 | 23.81 |
| 72 | 4002.72 | 20.69 |
| 73 | 4059.96 | 20.44 |
| 74 | 1186.59 | 22.31 |
| 75 | 1825.87 | 31.80 |
| 76 | 3401.66 | 23.42 |
| 77 | 1496.75 | 30.36 |
| 78 | 1832.92 | 31.91 |
| 79 | 2281.35 | 36.34 |
| 80 | 2344.34 | 33.66 |
| 81 | 3944.82 | 24.59 |
| 82 | 3002.23 | 23.80 |
| 83 | 3416.77 | 36.76 |
| 84 | 3501.86 | 31.79 |
| 85 | 6783.03 | 26.61 |
| 86 | 14111.27 | 21.97 |
| 87 | 2616.02 | 28.35 |
| 88 | 2810.45 | 36.73 |
| 89 | 2940.95 | 29.07 |
| 90 | 2946.45 | 34.96 |
| 91 | 1494.72 | 30.40 |
| 92 | 1080.53 | 27.86 |
| 93 | 2349.14 | 27.36 |
| 94 | 3303.00 | 23.07 |
| 95 | 4081.56 | 24.51 |
| 96 | 4670.27 | 25.84 |
| 97 | 4671.99 | 23.33 |
| 98 | 8933.94 | 22.57 |
| 99 | 1523.90 | 29.72 |
| 100 | 3956.82 | 25.20 |
| 101 | 3984.81 | 21.29 |
| 102 | 4830.78 | 26.61 |
| 103 | 3031.39 | 35.93 |
| 104 | 3788.76 | 25.21 |
| 105 | 2567.2 | 34.76 |
| 106 | 1447.8 | 19.49 |
| 107 | 2241.51 | 24.11 |
| 108 | 2461.11 | 30.84 |
| 109 | 1965.96 | 23.62 |
| 110 | 2189.08 | 27.17 |
| 111 | 1127.58 | 20.82 |
| 112 | 1400.71 | 20.35 |
| 113 | 1512.75 | 39.51 |
| 114 | 1860.53 | 34.24 |
| 115 | 1442.69 | 27.72 |
| 116 | 2590.78 | 27.96 |
| 117 | 1556.72 | 27.90 |
| 118 | 2309.15 | 21.95 |
| 119 | 2389.33 | 22.34 |
| 120 | 1478.68 | 39.28 |
| 121 | 1795.90 | 24.66 |
| 122 | 2211.03 | 35.06 |
| 123 | 1223.63 | 19.52 |
| 124 | 1829.04 | 21.22 |
| 125 | 1878.66 | 30.19 |
| 126 | 2009.96 | 32.27 |
| 127 | 2110.00 | 24.10 |
| 128 | 1552.79 | 29.75 |
| 129 | 1577.75 | 40.03 |
| 130 | 1936.94 | 34.71 |
| 131 | 2368.13 | 26.75 |
| 132 | 3633.05 | 33.25 |
| 133 | 1510.72 | 28.30 |
| 134 | 1668.87 | 40.49 |
| 135 | 2227.05 | 33.43 |
| 136 | 1495.75 | 39.41 |
| 137 | 1631.77 | 45.38 |
| 138 | 3158.60 | 29.62 |
| 139 | 1522.78 | 22.76 |
| 140 | 1727.87 | 39.61 |
| 141 | 1883.94 | 40.14 |
| 142 | 1460.71 | 19.83 |
| 143 | 1805.88 | 29.92 |
| 144 | 1898.93 | 40.30 |
| 145 | 2237.06 | 27.12 |
| 146 | 3178.33 | 30.26 |
| 147 | 1844.56 | 34.28 |
| 148 | 1378.57 | 37.16 |
| 149 | 1764.86 | 29.88 |
| 150 | 1791.88 | 30.77 |
| 151 | 2082.01 | 33.67 |
| 152 | 1768.90 | 20.77 |
| 153 | 3442.09 | 33.32 |
| 154 | 876.42 | 35.07 |
| 155 | 2352.14 | 26.74 |
| 156 | 937.50 | 34.12 |
| 157 | 1445.72 | 28.36 |
| 158 | 1893.10 | 28.85 |
| 159 | 2839.43 | 24.14 |
| 160 | 1600.76 | 29.61 |
| 161 | 1565.75 | 26.35 |
| 162 | 1627.76 | 29.47 |
| 163 | 1812.90 | 39.98 |
| 164 | 3137.52 | 30.29 |
| 165 | 1364.67 | 28.65 |
| 166 | 2298.07 | 33.82 |
| 167 | 3017.74 | 49.66 |
| 168 | 1235.61 | 26.67 |
| 169 | 1741.81 | 30.21 |
| 170 | 1818.90 | 30.93 |
| 171 | 1892.95 | 22.22 |
| 172 | 3280.69 | 22.69 |
| 173 | 2658.34 | 19.50 |
| 174 | 1407.71 | 27.46 |
| 175 | 1622.79 | 26.82 |
| 176 | 1684.78 | 29.64 |
| 177 | 1321.65 | 28.39 |
| 178 | 1350.68 | 27.13 |
| 179 | 1549.76 | 39.52 |
| 180 | 2233.10 | 22.47 |
| 181 | 2679.27 | 23.48 |
| 182 | 1835.79 | 20.02 |
| 183 | 3421.66 | 25.96 |
| 184 | 1708.85 | 30.44 |
| 185 | 1993.96 | 32.16 |
| 186 | 2695.31 | 23.46 |
| 187 | 1204.65 | 21.93 |
| 188 | 1467.86 | 24.38 |
| 189 | 1767.07 | 24.10 |
| 190 | 8176.30 | 19.57 |
| 191 | 1143.56 | 36.97 |
| 192 | 1834.90 | 31.05 |
| 193 | 2025.95 | 32.21 |
| 194 | 3489.70 | 31.45 |
| 195 | 1268.62 | 27.29 |
| 196 | 1659.82 | 29.35 |
| 197 | 2405.59 | 22.16 |
| 198 | 2483.21 | 27.54 |
| 199 | 2599.21 | 28.20 |
| 200 | 1750.86 | 23.80 |
| 201 | 1013.41 | 25.33 |
| 202 | 1016.49 | 25.88 |
| 203 | 1493.74 | 22.06 |
| 204 | 2104.04 | 32.97 |
| 205 | 3718.81 | 32.39 |
| 206 | 4251.98 | 28.66 |
| 207 | 4538.67 | 26.20 |
| 208 | 2067.93 | 20.68 |
| 209 | 2292.11 | 27.26 |
| 210 | 3702.39 | 32.39 |
| 211 | 965.46 | 27.84 |
| 212 | 2186.07 | 25.89 |
| 213 | 2584.29 | 35.08 |
| 214 | 2841.13 | 24.50 |
| 215 | 9866.78 | 20.85 |
| 216 | 1099.53 | 28.33 |
| 217 | 2471.25 | 34.69 |
| 218 | 3734.85 | 32.41 |
| 219 | 3927.86 | 33.50 |
| 220 | 3166.32 | 22.10 |
| 221 | 2339.08 | 33.95 |
| 222 | 2563.76 | 22.05 |
| 223 | 3219.35 | 35.00 |
| 224 | 3359.66 | 31.84 |
| 225 | 4097.98 | 24.59 |
| 226 | 4654.14 | 25.81 |
| 227 | 1584.77 | 29.72 |
| 228 | 2148.10 | 25.54 |
| 229 | 2639.45 | 21.33 |
| 230 | 3013.27 | 22.27 |
| 231 | 3205.39 | 19.71 |
| 232 | 3831.86 | 28.39 |
| 233 | 1050.52 | 27.03 |
| 234 | 2157.06 | 22.19 |
| 235 | 2407.16 | 27.65 |
| 236 | 2837.93 | 23.99 |
| 237 | 3058.02 | 30.20 |
| 238 | 1658.67 | 21.53 |
| 239 | 3311.32 | 24.46 |
| 240 | 3556.63 | 23.64 |
| 241 | 1085.50 | 21.70 |
| 242 | 1199.63 | 21.91 |
| 243 | 1247.58 | 22.00 |
| 244 | 1608.76 | 22.36 |
| 245 | 2501.20 | 34.30 |
| 246 | 3021.52 | 23.52 |
| 247 | 4153.75 | 33.41 |
| 248 | 5000.17 | 24.43 |
| 249 | 8917.48 | 22.53 |
| 250 | 4170.01 | 33.51 |
| 251 | 2235.13 | 34.10 |
| 252 | 2644.25 | 21.13 |
| 253 | 3943.96 | 33.53 |
| 254 | 11967.96 | 20.50 |
| 255 | 2553.23 | 34.14 |
| 256 | 1209.58 | 26.31 |
| 257 | 1899.94 | 21.41 |
| 258 | 1680.00 | 23.77 |
| 259 | 2195.06 | 20.15 |
| 260 | 3064.41 | 20.55 |
| 261 | 3554.07 | 31.11 |
| 262 | 3686.03 | 22.16 |
| 263 | 3802.12 | 33.10 |
| 264 | 4048.05 | 25.42 |
| 265 | 2380.16 | 36.48 |
| 266 | 1352.83 | 24.38 |
| 267 | 1638.80 | 20.26 |
| 268 | 2864.18 | 20.19 |
| 269 | 3754.66 | 37.16 |
| 270 | 4185.91 | 33.47 |
| 271 | 858.42 | 23.26 |
| 272 | 1159.64 | 26.05 |
| 273 | 1407.71 | 37.25 |
| 274 | 1439.72 | 29.62 |
| 275 | 1720.76 | 19.72 |
| 276 | 1846.93 | 32.04 |
| 277 | 3177.14 | 22.48 |
| 278 | 4113.80 | 24.58 |
| 279 | 2744.07 | 35.03 |
| 280 | 2767.26 | 21.52 |
| 281 | 1310.64 | 27.11 |
| 282 | 1613.88 | 23.95 |
| 283 | 1703.90 | 33.64 |
| 284 | 2761.40 | 21.46 |
| 285 | 3242.42 | 22.78 |
| 286 | 3338.17 | 23.36 |
| 287 | 3371.74 | 22.96 |
| 288 | 3593.53 | 20.25 |
| 289 | 3677.52 | 24.49 |
| 290 | 1624.80 | 30.81 |
| 291 | 2210.92 | 37.55 |
| 292 | 3290.37 | 24.12 |
| 293 | 4413.76 | 29.03 |
| 294 | 1482.73 | 22.47 |
| 295 | 1813.78 | 31.87 |
| 296 | 1934.87 | 20.04 |
| 297 | 2249.89 | 34.14 |
| 298 | 3280.59 | 25.76 |
| 299 | 1098.56 | 21.46 |
| 300 | 1125.58 | 21.76 |
| 301 | 1649.79 | 19.59 |
| 302 | 4025.68 | 20.73 |
| 303 | 980.33 | 35.59 |
| 304 | 1096.41 | 35.95 |
| 305 | 1698.65 | 37.60 |
| 306 | 2361.21 | 20.77 |
| 307 | 3148.50 | 24.22 |
| 308 | 3157.23 | 34.74 |
| 309 | 1304.59 | 27.95 |
| 310 | 3575.78 | 32.27 |
| 311 | 1510.75 | 20.12 |
| 312 | 2485.20 | 34.25 |
| 313 | 3076.33 | 19.64 |
| 314 | 3343.39 | 31.80 |
| 315 | 1405.71 | 20.16 |
| 316 | 2587.16 | 21.07 |
| 317 | 5213.25 | 22.47 |
| 318 | 2320.16 | 20.73 |
| 319 | 4491.89 | 26.23 |
| 320 | 10199.91 | 21.11 |
| 321 | 854.38 | 34.92 |
| 322 | 1084.56 | 36.85 |
| 323 | 1814.78 | 37.29 |
| 324 | 2078.05 | 22.47 |
| 325 | 2175.08 | 33.26 |
| 326 | 2411.78 | 26.97 |
| 327 | 3738.59 | 24.76 |
| 328 | 3935.57 | 34.15 |
| 329 | 4863.21 | 26.66 |
| 330 | 860.39 | 26.25 |
| 331 | 1567.78 | 20.23 |
| 332 | 2308.11 | 27.32 |
| 333 | 2923.77 | 36.44 |
| 334 | 3295.55 | 25.40 |
| 335 | 3870.85 | 33.39 |
| 336 | 1099.56 | 21.63 |
| 337 | 1359.70 | 22.92 |
| 338 | 2059.02 | 23.12 |
| 339 | 2077.03 | 21.78 |
| 340 | 3349.34 | 35.81 |
| 341 | 8853.85 | 21.08 |
| 342 | 1734.80 | 20.24 |
| 343 | 1847.95 | 43.93 |
| 344 | 2045.95 | 34.04 |
| 345 | 2289.47 | 33.56 |
| 346 | 2421.15 | 34.74 |
| 347 | 2480.67 | 23.00 |
| 348 | 2576.25 | 34.17 |
| 349 | 3353.93 | 23.53 |
| 350 | 1083.52 | 26.24 |
| 351 | 2073.17 | 27.43 |
| 352 | 7958.65 | 34.32 |
| 353 | 1837.88 | 30.54 |
| 354 | 2939.03 | 33.75 |
| 355 | 2977.31 | 19.59 |
| 356 | 3596.46 | 21.54 |
| 357 | 3851.68 | 24.97 |
| 358 | 1135.52 | 27.83 |
| 359 | 3378.05 | 38.81 |
| 360 | 3590.72 | 28.99 |
| 361 | 3959.80 | 19.95 |
| 362 | 1258.41 | 36.10 |
| 363 | 1513.50 | 36.82 |
| 364 | 1716.38 | 20.59 |
| 365 | 2022.97 | 33.38 |
| 366 | 2914.54 | 24.29 |
| 367 | 5527.56 | 27.58 |
| 368 | 931.51 | 20.00 |
| 369 | 973.26 | 35.59 |
| 370 | 1385.67 | 27.92 |
| 371 | 2272.31 | 23.80 |
| 372 | 4024.87 | 33.20 |
| 373 | 2216.11 | 33.79 |
| 374 | 2756.23 | 35.16 |
| 375 | 2777.71 | 21.55 |
| 376 | 3521.02 | 30.73 |
| 377 | 3750.72 | 32.45 |
| 378 | 4229.09 | 29.08 |
| 379 | 4846.50 | 26.40 |
| 380 | 1046.55 | 25.35 |
| 381 | 1608.80 | 30.94 |
| 382 | 1878.78 | 31.58 |
| 383 | 2589.16 | 22.45 |
| 384 | 4369.06 | 20.25 |
| 385 | 12717.08 | 26.92 |
| 386 | 1210.43 | 36.48 |
| 387 | 3092.54 | 36.22 |
| 388 | 3248.61 | 25.65 |
| 389 | 4012.41 | 20.81 |
| 390 | 11016.34 | 21.31 |
| 391 | 1284.61 | 29.17 |
| 392 | 1460.83 | 22.53 |
| 393 | 1807.88 | 23.98 |
| 394 | 2596.33 | 34.86 |
| 395 | 2686.97 | 29.06 |
| 396 | 3871.59 | 27.51 |
| 397 | 4069.63 | 25.30 |
| 398 | 4288.98 | 25.94 |
| 399 | 4426.21 | 20.09 |
| 400 | 1071.55 | 21.41 |
| 401 | 1749.88 | 30.54 |
| 402 | 1956.97 | 21.44 |
| 403 | 2189.12 | 26.54 |
| 404 | 2257.63 | 36.10 |
| 405 | 2917.54 | 28.99 |
| 406 | 3633.69 | 26.99 |
| 407 | 6055.77 | 21.03 |
| 408 | 6186.02 | 24.99 |
| 409 | 1858.92 | 24.17 |
| 410 | 2274.11 | 33.47 |
| 411 | 4522.51 | 26.20 |
| 412 | 6237.35 | 31.39 |
| 413 | 9883.82 | 20.84 |
| 414 | 3385.6 | 25.47 |
| 415 | 3745.6 | 26.65 |
| 416 | 1408.7 | 39.13 |
| 417 | 2551.3 | 34.75 |
| 418 | 3265.3 | 36.02 |
| 419 | 2739.3 | 28.4 |
| 420 | 2065 | 24.48 |
| 421 | 2264.1 | 22.67 |
| 422 | 1058.5 | 24.94 |
| 423 | 4467.9 | 29.05 |
| 424 | 2887.4 | 35.66 |
| 425 | 1635.8 | 40.33 |
| 426 | 2525.2 | 27.72 |
| 427 | 1526.8 | 23.63 |
| 428 | 1664.8 | 29.87 |
| 429 | 2583.3 | 28.31 |
| 430 | 2663.3 | 23.44 |
| 431 | 1878.9 | 42.18 |
| 432 | 1462.7 | 39.31 |
| 433 | 1834.9 | 24 |
| 434 | 1893.1 | 24.64 |
| 435 | 1934 | 21.63 |
| 436 | 1367.7 | 38.87 |
| 437 | 1009.5 | 27.33 |
| 438 | 3405.1 | 25.92 |
| 439 | 2314.1 | 33.67 |
| 440 | 3996.8 | 20.93 |
| 441 | 2823.6 | 29.07 |
| 442 | 1179.6 | 27.15 |
| 443 | 1435.7 | 28.86 |
| 444 | 2430.7 | 35.39 |
| 445 | 1134.6 | 23.68 |
| 446 | 2014 | 25.18 |
| 447 | 2577.3 | 24.55 |
| 448 | 1194.6 | 26.73 |
| 449 | 1588.8 | 30.2 |
| 450 | 2056 | 25.44 |
| 451 | 2442.16 | 34.11 |
| 452 | 1422.66 | 21.72 |
| 453 | 1623.8 | 24.15 |
| 454 | 1624.61 | 37.73 |
| 455 | 3298.48 | 36.06 |
| 456 | 1016.31 | 35.67 |
| 457 | 1580.94 | 24.31 |
| 458 | 1157.58 | 37.41 |
| 459 | 1250.61 | 27.94 |
| 460 | 1378.67 | 28.85 |
| 461 | 1392.68 | 21.75 |
| 462 | 1409.64 | 22.06 |
| 463 | 1425.65 | 22.34 |
| 464 | 1451.71 | 29.19 |
| 465 | 1576.66 | 26.5 |
| 466 | 1651.85 | 40.6 |
| 467 | 1876.94 | 22.29 |
| 468 | 1911.12 | 24.98 |
| 469 | 2064.01 | 21.95 |
| 470 | 2150.04 | 27.76 |
| 471 | 2751.59 | 29.16 |
| 472 | 4289.94 | 28.69 |
| 473 | 4306.05 | 28.78 |
| 474 | 4800.18 | 23.83 |
| 475 | 1111.32 | 35.47 |
| 476 | 1181.49 | 36.79 |
| 477 | 3168.38 | 24.69 |
| 478 | 1229.57 | 36.29 |
| 479 | 1579.78 | 29.83 |
| 480 | 1680.82 | 30.02 |
| 481 | 1725.66 | 38.3 |
| 482 | 5228.15 | 27.04 |
| 483 | 1769.78 | 28.25 |
| 484 | 1114.54 | 25.52 |
| 485 | 1390.5 | 37.05 |
| 486 | 2046.99 | 32.56 |
| 487 | 2899.33 | 49.62 |
| 488 | 1096.53 | 26.12 |
| 489 | 1257.49 | 34.26 |
| 490 | 868.45 | 23.35 |
| 491 | 1160.43 | 35.6 |
| 492 | 1539.8 | 40.36 |
| 493 | 3318.91 | 36.01 |
| 494 | 1084.48 | 25.31 |
| 495 | 1388.39 | 58.99 |
| 496 | 3129.86 | 35.93 |
| 497 | 1255.56 | 36.33 |
| 498 | 1383.69 | 39.02 |
| 499 | 1561.75 | 40.72 |
| 500 | 3108.55 | 31.25 |
| 501 | 1173.583 | 7.51 |
| 502 | 1100.55 | 36.99 |
| 503 | 1128.44 | 33.71 |
| 504 | 3149.6 | 31.22 |
| 505 | 1068.56 | 21.69 |
| 506 | 1349.48 | 36.47 |
| 507 | 1689.81 | 40.57 |
| 508 | 2305.7 | 34.8 |
| 509 | 840.44 | 23.94 |
| 510 | 911.3 | 34.39 |
| 511 | 1299.64 | 22.42 |
| 512 | 911.47 | 25.92 |
| 513 | 1025.51 | 25.44 |
| 514 | 3400.07 | 42.03 |
| 515 | 1901.89 | 43.92 |
| 516 | 1110.42 | 34.37 |
| 517 | 1032.5 | 25.89 |
| 518 | 1040.52 | 25.11 |
| 519 | 1265.64 | 27.14 |
| 520 | 1171.55 | 29.24 |
| 521 | 1012.53 | 35.08 |
| 522 | 1286.49 | 36.78 |
| 523 | 2932.36 | 34.11 |
| 524 | 1215.49 | 27.61 |
| 525 | 1423.68 | 21.47 |
| 526 | 1487.71 | 29.58 |

Preferably, markers 1-104 and/or 107-413 are employed.

With the present disclosure, it is also possible to determine the severity of the VD. This piece of information helps to decide what therapeutic measures are employed.

The migration time is determined by capillary electrophoresis (CE), for example, as set forth in the Example under item 2. Thus, a glass capillary of 90 cm in length and with an inner diameter (ID) of 75 μm and an outer diameter (OD) of 360 μm is operated at a voltage of 30 kV. As the solvent for the sample, 30% methanol, 0.5% formic acid in water is used.

It is known that the CE migration times may vary. Nevertheless, the order in which the polypeptide markers are eluted is typically the same for any CE system employed. In order to balance the differences in the migration time, the system may be normalized using standards for which the migration times are known. These standards may be, for example, the polypeptides stated in the Examples (see the Example, item 3).

### DETAILED DESCRIPTION OF THE PREFERRED EMBODIMENT

The characterization of the polypeptide markers shown in Tables 1 to 3 was determined by means of capillary electrophoresis-mass spectrometry (CE-MS), a method which has been described in detail, for example, by Neuhoff et al. (Rapid Communications in mass spectrometry, 2004, Vol. 20, pp. 149-156). The variation of the molecular masses between individual measurements or between different mass spectrometers is relatively small, typically within a range of ±0.1%, preferably within a range of ±0.05%, more preferably within a range of ±0.03%, even more preferably within a range of ±0.01%.

The polypeptide markers according to the disclosure are proteins or peptides or degradation products of proteins or peptides. They may be chemically modified, for example, by posttranslational modifications, such as glycosylation, phosphorylation, alkylation or disulfide bridges, or by other reactions, for example, within the scope of the degradation. In addition, the polypeptide markers may also be chemically altered, for example, oxidized, within the scope of the purification of the samples.

Proceeding from the parameters that determine the polypeptide markers (molecular weight and migration time), it is possible to identify the sequence of the corresponding polypeptides by methods known in the prior art.

The polypeptides according to the disclosure (see Tables 1 to 4) are used to diagnose the severity of the VD. “Diagnosis” means the process of knowledge gaining by assigning symptoms or phenomena to a disease or injury. In the present case, the severity of the VD is concluded from the presence or absence of particular polypeptide markers. Thus, the polypeptide markers according to the disclosure are determined in a sample from a subject, wherein its presence or absence allows to conclude the severity of the VD. The presence or absence of a polypeptide marker can be measured by any method known in the prior art. Methods which may be known are exemplified below.

A polypeptide marker is considered present if its measured value is at least as high as its threshold value. If the measured value is lower, then the polypeptide marker is considered absent. The threshold value can be determined either by the sensitivity of the measuring method (detection limit) or empirically.

In the context of the present disclosure, the threshold value is considered to be exceeded preferably if the measured value of the sample for a certain molecular mass is at least twice as high as that of a blank sample (for example, only buffer or solvent).

The polypeptide marker or markers is/are used in such a way that its/their presence or absence is measured, wherein the presence or absence is indicative of the severity of the VD (frequency marker). Thus, there are polypeptide markers which are typically present in subjects with VD, but occur less frequently or are absent in subjects with no VD, for example, 1-24 (Table 2). In addition, there are polypeptide markers which are present in patients with no VD, such as polypeptide markers No. 25 to 106, but are less frequently or not at all present in patients with VD.

TABLE 2
Polypeptide markers (frequency markers) for the diagnosis of vascular
diseases, their molecular masses and migration times, and their presence
and absence in patients suffering from VD (VD) and control groups
(control) as a factor (1 = 100%, 0 = 0%; sample processing and
measurement as described in the Example).

| | Occurrence | Occurrence |
|---|---|---|
| No. | Control | CVD |
| 1 | 0.01 | 0.58 |
| 2 | 0.18 | 0.75 |
| 3 | 0.14 | 0.67 |
| 4 | 0.25 | 0.75 |
| 5 | 0.08 | 0.58 |
| 6 | 0.05 | 0.54 |
| 7 | 0.36 | 0.83 |
| 8 | 0.31 | 0.79 |
| 9 | 0.26 | 0.71 |
| 10 | 0.14 | 0.58 |
| 11 | 0.06 | 0.50 |
| 12 | 0.15 | 0.58 |
| 13 | 0.31 | 0.75 |
| 14 | 0.32 | 0.75 |
| 15 | 0.12 | 0.54 |
| 16 | 0.03 | 0.46 |
| 17 | 0.25 | 0.67 |
| 18 | 0.18 | 0.58 |
| 19 | 0.47 | 0.88 |
| 20 | 0.18 | 0.58 |
| 21 | 0.09 | 0.50 |
| 22 | 0.27 | 0.67 |
| 23 | 0.18 | 0.58 |
| 24 | 0.10 | 0.50 |
| 25 | 0.44 | 0.04 |
| 26 | 0.44 | 0.04 |
| 27 | 0.44 | 0.04 |
| 28 | 0.48 | 0.08 |
| 29 | 0.74 | 0.33 |
| 30 | 0.62 | 0.21 |
| 31 | 0.45 | 0.04 |
| 32 | 0.41 | 0.00 |
| 33 | 0.62 | 0.21 |
| 34 | 0.74 | 0.33 |
| 35 | 0.41 | 0.00 |
| 36 | 0.78 | 0.38 |
| 37 | 0.45 | 0.04 |
| 38 | 0.70 | 0.29 |
| 39 | 0.78 | 0.38 |
| 40 | 0.41 | 0.00 |
| 41 | 0.67 | 0.25 |
| 42 | 0.59 | 0.17 |
| 43 | 0.67 | 0.25 |
| 44 | 0.54 | 0.13 |
| 45 | 0.63 | 0.21 |
| 46 | 0.42 | 0.00 |
| 47 | 0.80 | 0.38 |
| 48 | 0.54 | 0.13 |
| 49 | 0.42 | 0.00 |
| 50 | 0.58 | 0.17 |
| 51 | 0.42 | 0.00 |
| 52 | 0.72 | 0.29 |
| 53 | 0.52 | 0.08 |
| 54 | 0.52 | 0.08 |
| 55 | 0.59 | 0.17 |
| 56 | 0.65 | 0.21 |
| 57 | 0.48 | 0.04 |
| 58 | 0.57 | 0.13 |
| 59 | 0.56 | 0.13 |
| 60 | 0.44 | 0.00 |
| 61 | 0.57 | 0.13 |
| 62 | 0.44 | 0.00 |
| 63 | 0.73 | 0.29 |
| 64 | 0.53 | 0.08 |
| 65 | 0.50 | 0.04 |
| 66 | 0.49 | 0.04 |
| 67 | 0.70 | 0.25 |
| 68 | 0.91 | 0.46 |
| 69 | 0.71 | 0.25 |
| 70 | 0.63 | 0.17 |
| 71 | 0.47 | 0.00 |
| 72 | 0.81 | 0.33 |
| 73 | 0.59 | 0.13 |
| 74 | 0.90 | 0.42 |
| 75 | 0.77 | 0.29 |
| 76 | 0.94 | 0.46 |
| 77 | 0.69 | 0.21 |
| 78 | 0.49 | 0.00 |
| 79 | 0.66 | 0.17 |
| 80 | 0.53 | 0.04 |
| 81 | 0.53 | 0.04 |
| 82 | 0.67 | 0.17 |
| 83 | 0.50 | 0.00 |
| 84 | 0.50 | 0.00 |
| 85 | 0.62 | 0.13 |
| 86 | 0.88 | 0.38 |
| 87 | 0.63 | 0.13 |
| 88 | 0.55 | 0.04 |
| 89 | 0.52 | 0.00 |
| 90 | 0.61 | 0.08 |
| 91 | 0.91 | 0.38 |
| 92 | 0.75 | 0.21 |
| 93 | 0.67 | 0.13 |
| 94 | 0.73 | 0.17 |
| 95 | 0.60 | 0.04 |
| 96 | 0.69 | 0.13 |
| 97 | 0.85 | 0.29 |
| 98 | 0.60 | 0.04 |
| 99 | 0.70 | 0.13 |
| 100 | 0.71 | 0.13 |
| 101 | 0.58 | 0.00 |
| 102 | 0.59 | 0.00 |
| 103 | 0.80 | 0.17 |
| 104 | 0.77 | 0.08 |
| 105 | 0.98 | 0.49 |
| 106 | 0.90 | 0.49 |

In addition or also alternatively to the frequency markers (determination of presence or absence), the amplitude markers as stated in Table 3 may also be used for the diagnosis of VD (Nos. 107-526). Amplitude markers are used in such a way that the presence or absence is not critical, but the height of the signal (the amplitude) decides if the signal is present in both groups. In Tables 3 and 4, the mean amplitudes of the corresponding signals (characterized by mass and migration time) averaged over all samples measured are stated. Two normalization methods are possible to achieve comparability between differently concentrated samples or different measuring methods. In the first approach, all peptide signals of a sample are normalized to a total amplitude of 1 million counts. Therefore, the respective mean amplitudes of the individual markers are stated as parts per million (ppm). The amplitude markers obtained by this method are shown in Table 3 (Nos. 107-413).

In addition, it is possible to define further amplitude markers by an alternative normalization method: In this case, all peptide signals of one sample are scaled with a common normalization factor. Thus, a linear regression is formed between the peptide amplitudes of the individual samples and the reference values of all known polypeptides. The slope of the regression line just corresponds to the relative concentration and is used as a normalization factor for this sample. The biomarkers obtained by this normalization method are shown in Table 4 (Nos. 414-526).

All groups employed consist of at least 20 individual patient or control samples in order to obtain reliable mean amplitudes. The decision for a diagnosis (VD or not) is made as a function of how high the amplitude of the respective polypeptide markers in the patient sample is in comparison with the mean amplitudes in the control groups or the VD group. If the amplitude rather corresponds to the mean amplitudes of the VD group, the existence of a vascular disease is to be considered, and if it rather corresponds to the mean amplitudes of the control group, the non-existence of VD is to be considered. The distance between the measured value and the mean amplitude can be considered a probability of the sample's belonging to a certain group. An exemplary explanation shall be given by means of marker No. 137 (Table 3). The mean amplitude of the marker is significantly increased in VD (12044 ppm vs. 5726 ppm in the control group). Now, if the value for this marker in a patient sample is from 0 to 5726 ppm or exceeds this range by a maximum of 20%, i.e., from 0 to 6871 ppm, then this sample belongs to the control group. If the value is 12044 ppm or up to 20% below, or higher, i.e., between 9635 and very high values, this is to be considered an indication of a vascular disease.

Alternatively, the distance between the measured value and the mean amplitude may be considered a probability of the sample's belonging to a certain group. A frequency marker is a variant of an amplitude marker in which the amplitude is low in some samples. It is possible to convert such frequency markers to amplitude markers by including the corresponding samples in which the marker is not found into the calculation of the amplitude with a very small amplitude, on the order of the detection limit.

TABLE 3
Amplitude markers with normalization according to approach 1

| | Mean | Mean |
|---|---|---|
| | Amplitude | Amplitude |
| No. | Control Group | CVD Group |
| 107 | 94 | 253 |
| 108 | 116 | 233 |
| 109 | 50 | 123 |
| 110 | 766 | 1878 |
| 111 | 45 | 175 |
| 112 | 89 | 419 |
| 113 | 69 | 146 |
| 114 | 174 | 418 |
| 115 | 78 | 689 |
| 116 | 47 | 99 |
| 117 | 59 | 188 |
| 118 | 120 | 357 |
| 119 | 317 | 2460 |
| 120 | 121 | 463 |
| 121 | 172 | 380 |
| 122 | 796 | 1674 |
| 123 | 167 | 888 |
| 124 | 1703 | 636 |
| 125 | 768 | 3651 |
| 126 | 340 | 1283 |
| 127 | 193 | 583 |
| 128 | 135 | 320 |
| 129 | 243 | 566 |
| 130 | 95 | 214 |
| 131 | 161 | 768 |
| 132 | 118 | 299 |
| 133 | 116 | 267 |
| 134 | 840 | 1950 |
| 135 | 102 | 288 |
| 136 | 127 | 283 |
| 137 | 5726 | 12044 |
| 138 | 263 | 728 |
| 139 | 506 | 154 |
| 140 | 113 | 289 |
| 141 | 150 | 301 |
| 142 | 136 | 290 |
| 143 | 51 | 189 |
| 144 | 168 | 343 |
| 145 | 196 | 769 |
| 146 | 119 | 250 |
| 147 | 161 | 358 |
| 148 | 227 | 58 |
| 149 | 130 | 289 |
| 150 | 97 | 196 |
| 151 | 192 | 504 |
| 152 | 301 | 128 |
| 153 | 442 | 108 |
| 154 | 154 | 1119 |
| 155 | 197 | 725 |
| 156 | 82 | 201 |
| 157 | 100 | 202 |
| 158 | 189 | 513 |
| 159 | 1054 | 486 |
| 160 | 161 | 412 |
| 161 | 123 | 456 |
| 162 | 229 | 517 |
| 163 | 273 | 554 |
| 164 | 196 | 517 |
| 165 | 197 | 97 |
| 166 | 176 | 506 |
| 167 | 1480 | 686 |
| 168 | 3107 | 880 |
| 169 | 80 | 216 |
| 170 | 203 | 1328 |
| 171 | 344 | 848 |
| 172 | 797 | 206 |
| 173 | 1146 | 2842 |
| 174 | 224 | 568 |
| 175 | 138 | 450 |
| 176 | 258 | 525 |
| 177 | 15571 | 7296 |
| 178 | 367 | 745 |
| 179 | 185 | 575 |
| 180 | 260 | 130 |
| 181 | 1492 | 3433 |
| 182 | 784 | 351 |
| 183 | 1158 | 2826 |
| 184 | 919 | 371 |
| 185 | 156 | 466 |
| 186 | 1694 | 4348 |
| 187 | 201 | 66 |
| 188 | 1787 | 737 |
| 189 | 2810 | 6060 |
| 190 | 1703 | 766 |
| 191 | 461 | 1095 |
| 192 | 707 | 6865 |
| 193 | 493 | 1490 |
| 194 | 346 | 799 |
| 195 | 3338 | 9120 |
| 196 | 240 | 654 |
| 197 | 80 | 203 |
| 198 | 490 | 236 |
| 199 | 259 | 533 |
| 200 | 1142 | 421 |
| 201 | 1241 | 2506 |
| 202 | 1511 | 749 |
| 203 | 294 | 107 |
| 204 | 1090 | 2230 |
| 205 | 1151 | 456 |
| 206 | 983 | 475 |
| 207 | 220 | 44 |
| 208 | 1195 | 546 |
| 209 | 3909 | 1825 |
| 210 | 406 | 167 |
| 211 | 149 | 58 |
| 212 | 1098 | 2400 |
| 213 | 769 | 164 |
| 214 | 527 | 1675 |
| 215 | 1173 | 416 |
| 216 | 711 | 324 |
| 217 | 470 | 181 |
| 218 | 723 | 333 |
| 219 | 213 | 102 |
| 220 | 345 | 169 |
| 221 | 677 | 334 |
| 222 | 2489 | 744 |
| 223 | 132 | 63 |
| 224 | 1451 | 717 |
| 225 | 1324 | 299 |
| 226 | 1689 | 741 |
| 227 | 88 | 238 |
| 228 | 191 | 701 |
| 229 | 361 | 118 |
| 230 | 5095 | 1789 |
| 231 | 601 | 241 |
| 232 | 1200 | 403 |
| 233 | 736 | 245 |
| 234 | 1171 | 297 |
| 235 | 678 | 263 |
| 236 | 1597 | 482 |
| 237 | 115 | 353 |
| 238 | 392 | 146 |
| 239 | 120 | 265 |
| 240 | 1127 | 465 |
| 241 | 623 | 140 |
| 242 | 250 | 115 |
| 243 | 633 | 306 |
| 244 | 224 | 81 |
| 245 | 120 | 60 |
| 246 | 1275 | 438 |
| 247 | 283 | 89 |
| 248 | 1514 | 737 |
| 249 | 264 | 91 |
| 250 | 900 | 278 |
| 251 | 776 | 338 |
| 252 | 411 | 97 |
| 253 | 227 | 103 |
| 254 | 186 | 53 |
| 255 | 890 | 332 |
| 256 | 469 | 1170 |
| 257 | 152 | 70 |
| 258 | 21200 | 7156 |
| 259 | 282 | 115 |
| 260 | 474 | 152 |
| 261 | 117 | 274 |
| 262 | 1359 | 517 |
| 263 | 421 | 195 |
| 264 | 183 | 83 |
| 265 | 151 | 64 |
| 266 | 206 | 99 |
| 267 | 588 | 256 |
| 268 | 304 | 119 |
| 269 | 147 | 61 |
| 270 | 172 | 66 |
| 271 | 338 | 157 |
| 272 | 292 | 138 |
| 273 | 227 | 110 |
| 274 | 142 | 387 |
| 275 | 166 | 79 |
| 276 | 179 | 385 |
| 277 | 200 | 75 |
| 278 | 211 | 81 |
| 279 | 169 | 68 |
| 280 | 359 | 157 |
| 281 | 141 | 284 |
| 282 | 244 | 104 |
| 283 | 882 | 331 |
| 284 | 903 | 324 |
| 285 | 231 | 98 |
| 286 | 1420 | 457 |
| 287 | 2096 | 591 |
| 288 | 676 | 261 |
| 289 | 470 | 234 |
| 290 | 169 | 49 |
| 291 | 234 | 517 |
| 292 | 624 | 309 |
| 293 | 279 | 111 |
| 294 | 444 | 130 |
| 295 | 752 | 1640 |
| 296 | 543 | 191 |
| 297 | 164 | 66 |
| 298 | 785 | 274 |
| 299 | 185 | 79 |
| 300 | 234 | 99 |
| 301 | 179 | 46 |
| 302 | 360 | 141 |
| 303 | 106 | 37 |
| 304 | 146 | 47 |
| 305 | 730 | 323 |
| 306 | 373 | 40 |
| 307 | 97 | 39 |
| 308 | 48 | 104 |
| 309 | 176 | 375 |
| 310 | 87 | 185 |
| 311 | 187 | 91 |
| 312 | 143 | 43 |
| 313 | 381 | 143 |
| 314 | 402 | 194 |
| 315 | 237 | 113 |
| 316 | 519 | 207 |
| 317 | 115 | 56 |
| 318 | 197 | 61 |
| 319 | 254 | 1335 |
| 320 | 283 | 140 |
| 321 | 88 | 201 |
| 322 | 119 | 56 |
| 323 | 129 | 46 |
| 324 | 125 | 50 |
| 325 | 3240 | 7677 |
| 326 | 114 | 51 |
| 327 | 236 | 89 |
| 328 | 163 | 79 |
| 329 | 702 | 204 |
| 330 | 481 | 159 |
| 331 | 407 | 175 |
| 332 | 228 | 79 |
| 333 | 200 | 98 |
| 334 | 356 | 80 |
| 335 | 152 | 72 |
| 336 | 178 | 64 |
| 337 | 281 | 50 |
| 338 | 293 | 104 |
| 339 | 796 | 299 |
| 340 | 174 | 83 |
| 341 | 1025 | 194 |
| 342 | 209 | 95 |
| 343 | 407 | 145 |
| 344 | 144 | 462 |
| 345 | 182 | 74 |
| 346 | 95 | 42 |
| 347 | 92 | 16 |
| 348 | 150 | 36 |
| 349 | 256 | 96 |
| 350 | 130 | 51 |
| 351 | 96 | 46 |
| 352 | 330 | 157 |
| 353 | 248 | 107 |
| 354 | 205 | 69 |
| 355 | 310 | 36 |
| 356 | 411 | 139 |
| 357 | 789 | 179 |
| 358 | 263 | 104 |
| 359 | 184 | 84 |
| 360 | 206 | 52 |
| 361 | 755 | 287 |
| 362 | 246 | 26 |
| 363 | 316 | 106 |
| 364 | 1329 | 142 |
| 365 | 122 | 23 |
| 366 | 105 | 46 |
| 367 | 311 | 127 |
| 368 | 131 | 56 |
| 369 | 206 | 38 |
| 370 | 104 | 41 |
| 371 | 126 | 43 |
| 372 | 345 | 110 |
| 373 | 416 | 151 |
| 374 | 209 | 86 |
| 375 | 268 | 54 |
| 376 | 549 | 188 |
| 377 | 115 | 36 |
| 378 | 353 | 110 |
| 379 | 379 | 135 |
| 380 | 503 | 52 |
| 381 | 753 | 360 |
| 382 | 335 | 2617 |
| 383 | 97 | 31 |
| 384 | 1280 | 178 |
| 385 | 438 | 64 |
| 386 | 374 | 92 |
| 387 | 329 | 109 |
| 388 | 283 | 124 |
| 389 | 273 | 36 |
| 390 | 3045 | 864 |
| 391 | 51 | 25 |
| 392 | 711 | 69 |
| 393 | 187 | 82 |
| 394 | 74 | 29 |
| 395 | 197 | 61 |
| 396 | 320 | 100 |
| 397 | 712 | 156 |
| 398 | 187 | 48 |
| 399 | 337 | 7 |
| 400 | 133 | 59 |
| 401 | 297 | 110 |
| 402 | 164 | 55 |
| 403 | 876 | 4574 |
| 404 | 820 | 309 |
| 405 | 845 | 288 |
| 406 | 475 | 119 |
| 407 | 275 | 91 |
| 408 | 1590 | 291 |
| 409 | 1343 | 334 |
| 410 | 180 | 61 |
| 411 | 149 | 13 |
| 412 | 298 | 135 |
| 413 | 469 | 42 |

TABLE 4
Amplitude markers with normalization according to approach 2

| | Mean Amplitude | Mean Amplitude |
|---|---|---|
| No. | Control Group | CVD Group |
| 414 | 3214 | 2678 |
| 415 | 514 | 250 |
| 416 | 1359 | 615 |
| 417 | 581 | 174 |
| 418 | 630 | 499 |
| 419 | 212 | 141 |
| 420 | 681 | 381 |
| 421 | 445 | 227 |
| 422 | 1178 | 103 |
| 423 | 540 | 348 |
| 424 | 188 | 206 |
| 425 | 1540 | 687 |
| 426 | 569 | 914 |
| 427 | 301 | 106 |
| 428 | 976 | 511 |
| 429 | 972 | 515 |
| 430 | 1320 | 729 |
| 431 | 278 | 210 |
| 432 | 1682 | 1196 |
| 433 | 589 | 287 |
| 434 | 384 | 502 |
| 435 | 1006 | 399 |
| 436 | 1064 | 800 |
| 437 | 270 | 216 |
| 438 | 3453 | 2235 |
| 439 | 837 | 790 |
| 440 | 1353 | 684 |
| 441 | 710 | 733 |
| 442 | 809 | 627 |
| 443 | 8328 | 3904 |
| 444 | 596 | 661 |
| 445 | 380 | 593 |
| 446 | 2389 | 1375 |
| 447 | 297 | 285 |
| 448 | 4154 | 2314 |
| 449 | 532 | 953 |
| 450 | 1145 | 733 |
| 451 | 744 | 845 |
| 452 | 2878 | 2433 |
| 453 | 8725 | 4307 |
| 454 | 1109 | 1620 |
| 455 | 750 | 409 |
| 456 | 687 | 971 |
| 457 | 2155 | 1229 |
| 458 | 1622 | 1964 |
| 459 | 50512 | 45582 |
| 460 | 2259 | 2915 |
| 461 | 4142 | 4664 |
| 462 | 6265 | 8932 |
| 463 | 1969 | 2931 |
| 464 | 36818 | 19376 |
| 465 | 1352 | 1591 |
| 466 | 5789 | 1562 |
| 467 | 2562 | 1358 |
| 468 | 91735 | 90455 |
| 469 | 7723 | 4053 |
| 470 | 2496 | 1342 |
| 471 | 358 | 423 |
| 472 | 3360 | 3317 |
| 473 | 4000 | 2575 |
| 474 | 1388 | 2785 |
| 475 | 335 | 3122 |
| 476 | 302 | 237 |
| 477 | 351 | 847 |
| 478 | 186 | 145 |
| 479 | 3094 | 2397 |
| 480 | 4737 | 2446 |
| 481 | 1468 | 2644 |
| 482 | 566 | 974 |
| 483 | 535 | 429 |
| 484 | 2818 | 4530 |
| 485 | 17423 | 37226 |
| 486 | 3087 | 1793 |
| 487 | 25 | 319 |
| 488 | 3397 | 6633 |
| 489 | 2904 | 6138 |
| 490 | 239 | 198 |
| 491 | 1794 | 3083 |
| 492 | 2558 | 1701 |
| 493 | 428 | 419 |
| 494 | 1326 | 2891 |
| 495 | 181 | 788 |
| 496 | 212 | 207 |
| 497 | 741 | 600 |
| 498 | 135 | 197 |
| 499 | 4632 | 4647 |
| 500 | 331 | 461 |
| 501 | 302 | 414 |
| 502 | 206 | 306 |
| 503 | 1521 | 3346 |
| 504 | 349 | 561 |
| 505 | 211 | 315 |
| 506 | 208 | 247 |
| 507 | 1270 | 1039 |
| 508 | 305 | 334 |
| 509 | 213 | 266 |
| 510 | 2436 | 3827 |
| 511 | 460 | 294 |
| 512 | 389 | 924 |
| 513 | 197 | 273 |
| 514 | 152 | 448 |
| 515 | 743 | 575 |
| 516 | 428 | 713 |
| 517 | 186 | 298 |
| 518 | 219 | 296 |
| 519 | 4218 | 7618 |
| 520 | 75 | 148 |
| 521 | 96 | 214 |
| 522 | 56 | 92 |
| 523 | 208 | 316 |
| 524 | 349 | 729 |
| 525 | 543 | 1220 |
| 526 | 388 | 684 |

The subject from whom the sample in which the presence or absence of one or more polypeptide markers is determined is derived may be any subject which is capable of suffering from VD. Preferably, the subject is a mammal, and most preferably, it is a human.

In a preferred embodiment of the disclosure, not just one polypeptide marker but a combination of polypeptide markers is used to determine the severity of VD, wherein the severity of VD can be concluded from their presence or absence. By comparing a plurality of polypeptide markers, a bias in the overall result from a few individual deviations from the typical presence probability in the sick or control individual can be reduced or avoided.

The sample in which the presence or absence of the peptide marker or markers according to the disclosure is measured may be any sample which is obtained from the body of the subject. The sample is a sample which has a polypeptide composition suitable for providing information about the state of the subject (VD or not). For example, it may be blood, urine, synovial fluid, a tissue fluid, a body secretion, sweat, cerebrospinal fluid, lymph, intestinal, gastric or pancreatic juice, bile, lacrimal fluid, a tissue sample, sperm, vaginal fluid or a feces sample. Preferably, it is a liquid sample. In a preferred embodiment, the sample is a urine sample or blood sample, wherein a blood sample may be a (blood) serum or (blood) plasma sample.

Urine samples can be taken as preferred in the prior art. Preferably, a midstream urine sample is used as said urine sample in the context of the present disclosure. For example, the urine sample may also be taken by means of a urination apparatus as described in WO 01/74275.

Blood samples can be taken by methods known in the prior art, for example, from a vein, artery or capillary. Usually, a blood sample is obtained by withdrawing venous blood by means of a syringe, for example, from an arm of the subject. The term “blood sample” includes samples obtained from blood by further purification and separation methods, such as blood plasma or blood serum.

The presence or absence of a polypeptide marker in the sample may be determined by any method known in the prior art that is suitable for measuring polypeptide markers. Such methods are known to the skilled person. In principle, the presence or absence of a polypeptide marker can be determined by direct methods, such as mass spectrometry, or indirect methods, for example, by means of ligands.

If required or desirable, the sample from the subject, for example, the urine or blood sample, may be pretreated by any suitable means and, for example, purified or separated before the presence or absence of the polypeptide marker or markers is measured. The treatment may comprise, for example, purification, separation, dilution or concentration. The methods may be, for example, centrifugation, filtration, ultrafiltration, dialysis, precipitation or chromatographic methods, such as affinity separation or separation by means of ion-exchange chromatography, electrophoretic separation, i.e., separation by different migration behaviors of electrically charged particles in solution upon application of an electric field. Particular examples thereof are gel electrophoresis, two-dimensional polyacrylamide gel electrophoresis (2D-PAGE), capillary electrophoresis, metal affinity chromatography, immobilized metal affinity chromatography (IMAC), lectin-based affinity chromatography, liquid chromatography, high-performance liquid chromatography (HPLC), normal and reverse-phase HPLC, cation-exchange chromatography and selective binding to surfaces. All these methods are well known to the skilled person, and the skilled person will be able to select the method as a function of the sample employed and the method for determining the presence or absence of the polypeptide marker or markers.

In one embodiment of the disclosure, the sample, before being separated by capillary electrophoresis, is separated, purified by ultracentrifugation and/or divided by ultrafiltration into fractions which contain polypeptide markers of a particular molecular size.

Preferably, a mass-spectrometric method is used to determine the presence or absence of a polypeptide marker, wherein a purification or separation of the sample may be performed upstream from such method. As compared to the currently employed methods, mass-spectrometric analysis has the advantage that the concentration of many (>100) polypeptides of a sample can be determined by a single analysis. Any type of mass spectrometer may be employed. By means of mass spectrometry, it is possible to measure 10 fmol of a polypeptide marker, i.e., 0.1 ng of a 10 kD protein, as a matter of routine with a measuring accuracy of about ±0.01% in a complex mixture. In mass spectrometers, an ion-forming unit is coupled with a suitable analytic device. For example, electrospray-ionization (ESI) interfaces are mostly used to measure ions in liquid samples, whereas MALDI (matrix-assisted laser desorption/ionization) is used for measuring ions from a sample crystallized in a matrix. To analyze the ions formed, quadrupoles, ion traps or time-of-flight (TOF) analyzers may be used, for example.

In electrospray ionization (ESI), the molecules present in solution are atomized, inter alia, under the influence of high voltage (e.g., 1-8 kV), which forms charged droplets at first that become smaller from the evaporation of the solvent. Finally, so-called Coulomb explosions result in the formation of free ions, which can then be analyzed and detected.

In the analysis of the ions by means of TOF, a particular acceleration voltage is applied which confers an equal amount of kinetic energy to the ions. Thereafter, the time that the respective ions take to travel a particular drifting distance through the flying tube is measured very accurately. Since with equal amounts of kinetic energy, the velocity of the ions depends on their mass, the latter can thus be determined. TOF analyzers have a very high scanning speed and therefore reach a good resolution. Preferred methods for the determination of the presence and absence of polypeptide markers include gas-phase ion spectrometry, such as laser desorption/ionization mass spectrometry, MALDI-TOF MS, SELDI-TOF MS (surface-enhanced laser desorption/ionization), LC MS (liquid chromatography/mass spectrometry), 2D-PAGE/MS and capillary electrophoresis-mass spectrometry (CE-MS). All the methods mentioned are known to the skilled person.

A particularly preferred method is CE-MS, in which capillary electrophoresis is coupled with mass spectrometry. This method has been described in some detail, for example, in the German Patent Application DE 10021737, in Kaiser et al. (J. Chromatogr A, 2003, Vol. 1013: 157-171, and Electrophoresis, 2004, 25: 2044-2055) and in Wittke et al. (J. Chromatogr. A, 2003, 1013: 173-181). The CE-MS technology allows the determination if the presence of some hundreds of polypeptide markers of a sample simultaneously within a short time and in a small volume with high sensitivity. After a sample has been measured, a pattern of the measured polypeptide markers is prepared, and this pattern can be compared with reference patterns of a sick or healthy subjects. In most cases, it is sufficient to use a limited number of polypeptide markers for the diagnosis of UAS. A CE-MS method which includes CE coupled on-line to an ESI-TOF MS is further preferred.

For CE-MS, the use of volatile solvents is preferred, and it is best to work under essentially salt-free conditions. Examples of such solvents include acetonitrile, isopropanol, methanol and the like. The solvents can be diluted with water or a weak acid (e.g., 0.1% to 1% formic acid) in order to protonate the analyte, preferably the polypeptides.

By means of capillary electrophoresis, it is possible to separate molecules by their charge and size. Neutral particles will migrate at the speed of the electroosmotic flow upon application of a current, while cations are accelerated towards the cathode, and anions are delayed. The advantage of the capillaries in electrophoresis resides in the favorable ratio of surface to volume, which enables a good dissipation of the Joule heat generated during the current flow. This in turn allows high voltages (usually up to 30 kV) to be applied and thus a high separating performance and short times of analysis.

In capillary electrophoresis, silica glass capillaries having inner diameters of typically from 50 to 75 μm are usually employed. The lengths employed are, for example, 30-100 cm. In addition, the separating capillaries are usually made of plastic-coated silica glass. The capillaries may be either untreated, i.e., expose their hydrophilic groups on the interior surface, or coated on the interior surface. A hydrophobic coating may be used to improve the resolution. In addition to the voltage, a pressure may also be applied, which typically is within a range of from 0 to 1 psi. The pressure may also be applied only during the separation or altered meanwhile.

In a preferred method for measuring polypeptide markers, the markers of the sample are separated by capillary electrophoresis, then directly ionized and transferred on-line into a coupled mass spectrometer for detection.

In the method according to the disclosure, it is advantageous to use several polypeptide markers for diagnosing the VD. In particular, at least three polypeptide markers may be used, for example, markers 1, 2 and 3; 1, 2 and 4; etc.

The use of at least 4, 5 or 6 markers is more preferred.

The use of at least 11 markers, for example, markers 1 to 11, is even more preferred.

The use of all the 526 markers stated in Tables 1 to 4 is most preferred.

In order to determine the probability of the existence of a severe VD when several markers are used, statistic methods known to the skilled person may be used. For example, the Random Forests method described by Weissinger et al. (Kidney Int., 2004, 65: 2426-2434) may be used by using a computer program such as S-Plus, or the support vector machines as described in the same publication.

### EXAMPLE

### 1. Sample Preparation

For detecting the polypeptide markers for diagnosing the VD, urine was employed. Urine was collected from healthy donors (control group) as well as from patients suffering from severe VD.

For the subsequent CE-MS measurement, the proteins which are also contained in the urine of patients in an elevated concentration, such as albumin and immunoglobulins, had to be separated off by ultrafiltration. Thus, 700 μl of urine was collected and admixed with 700 μm of filtration buffer (2 M urea, 10 mM ammonia, 0.02% SDS). This 1.4 ml of sample volume was ultrafiltrated (20 kDa, Sartorius, Gottingen, Germany). The ultrafiltration was performed at 3000 rpm in a centrifuge until 1.1 ml of ultrafiltrate was obtained.

The 1.1 ml of filtrate obtained was then applied to a PD 10 column (Amersham Bioscience, Uppsala, Sweden) and eluted with 2.5 ml of 0.01% NH₄OH, and lyophilized. For the CE-MS measurement, the polypeptides were then resuspended with 20 μl of water (HPLC grade, Merck).

### 2. CE-MS Measurement

The CE-MS measurements were performed with a capillary electrophoresis system from Beckman Coulter (P/ACE MDQ System; Beckman Coulter Inc., Fullerton, Calif., USA) and an ESI-TOF mass spectrometer from Bruker (micro-TOF MS, Bruker Daltonik, Bremen, Germany).

The CE capillaries were supplied by Beckman Coulter and had an ID/OD of 50/360 μm and a length of 90 cm. The mobile phase for the CE separation consisted of 20% acetonitrile and 0.25% formic acid in water. For the “sheath flow” on the MS, 30% isopropanol with 0.5% formic acid was used, here at a flow rate of 2 μl/min. The coupling of CE and MS was realized by a CE-ESI-MS Sprayer Kit (Agilent Technologies, Waldbronn, Germany).

For injecting the sample, a pressure of from 1 to a maximum of 6 psi was applied, and the duration of the injection was 99 seconds. With these parameters, about 150 nl of the sample was injected into the capillary, which corresponds to about 10% of the capillary volume. A stacking technique was used to concentrate the sample in the capillary. Thus, before the sample was injected, a 1 M NH₃ solution was injected for 7 seconds (at 1 psi), and after the sample was injected, a 2 M formic acid solution was injected for 5 seconds. When the separation voltage (30 kV) was applied, the analytes were automatically concentrated between these solutions.

The subsequent CE separation was performed with a pressure method: 40 minutes at 0 psi, then 0.1 psi for 2 min, 0.2 psi for 2 min, 0.3 psi for 2 min, 0.4 psi for 2 min, and finally 0.5 psi for 32 min. The total duration of a separation run was thus 80 minutes.

In order to obtain as good a signal intensity as possible on the side of the MS, the nebulizer gas was turned to the lowest possible value. The voltage applied to the spray needle for generating the electrospray was 3700-4100 V. The remaining settings at the mass spectrometer were optimized for peptide detection according to the manufacturer's instructions. The spectra were recorded over a mass range of m/z 400 to m/z 3000 and accumulated every 3 seconds.

### 3. Standards for the CE Measurement

For checking and standardizing the CE measurement, the following proteins or polypeptides which are characterized by the stated CE migration times were employed:

| | Migration |
|---|---|
| Protein/polypeptide | time |
| Aprotinin (SIGMA, Taufkirchen, | 9.2 min |
| DE, Cat. # Al 153) | |
| Ribonuclease (SIGMA, Taufkirchen, | 10.9 min |
| DE, Cat. # R4875) | |
| Lysozyme (SIGMA, Taufkirchen, | 8.9 min |
| DE, Cat. # L7651) | |
| “REV”, Sequence: REVQSKIGYGRQIIS | 15.6 min |
| “ELM”, Sequence: ELMTGELPYSHINNR | 23.4 min |
| DQIIFMVGR | |
| “KINCON”, Sequence: TGSLPYSHIGSR | 20.0 min |
| DQIIFMVGR | |
| “GIVLY” Sequence: GIVLYELMTGELPYSHIN | 36.8 min |

The proteins/polypeptides were employed at a concentration of 10 pmol/μl each in water. “REV”, “ELM, “KINCON” and “GIVLY” are synthetic peptides.

The molecular masses of the peptides and the m/z ratios of the individual charge states visible in MS are stated in the following Table:

| | 1.0079 | 1.0079 | 1.0079 | 1.0079 | 1.0079 | 1.0079 | 1.0079 |
|---|---|---|---|---|---|---|---|
| H | Aprotinin | Ribonuclease | Lysozym | REV | KINCON | ELM | GIVLY |
| mono | Mono | Mono | Mono | Mono | Mono | Mono | Mono |
| m/z | Mass | Mass | Mass | Mass | Mass | Mass | Mass |
| 0 | 6513.0900 | 13681.3200 | 14303.8800 | 1732.9600 | 2333.1900 | 2832.4100 | 2048.0300 |
| 1 | 6514.0979 | 13682.3279 | 14304.8879 | 1733.9679 | 2334.1979 | 2833.4179 | 2049.0379 |
| 2 | 3257.5529 | 6841.6679 | 7152.9479 | 867.4879 | 1167.6029 | 1417.2729 | 1025.0229 |
| 3 | 2172.0379 | 4561.4479 | 4768.9679 | 578.6612 | 778.7379 | 945.1446 | 683.6846 |
| 4 | 1629.2804 | 3421.3379 | 3576.9779 | 434.2479 | 584.3054 | 709.1104 | 513.0154 |
| 5 | 1303.6259 | 2737.2719 | 2861.7839 | 347.5999 | 467.6459 | 567.4899 | 410.6139 |
| 6 | 1086.5229 | 2281.2279 | 2384.9879 | 289.8346 | 389.8729 | 473.0762 | 342.3462 |
| 7 | 931.4494 | 1955.4822 | 2044.4193 | 248.5736 | 334.3208 | 405.6379 | 293.5836 |
| 8 | 815.1442 | 1711.1729 | 1788.9929 | 217.6279 | 292.6567 | 355.0592 | 257.0117 |
| 9 | 724.6846 | 1521.1546 | 1590.3279 | 193.5590 | 260.2512 | 315.7201 | 228.5668 |
| 10 | 652.3169 | 1369.1399 | 1431.3959 | 174.3039 | 234.3269 | 284.2489 | 205.8109 |
| 11 | 593.1070 | 1244.7643 | 1301.3606 | 158.5497 | 213.1161 | 258.4997 | 187.1924 |
| 12 | 543.7654 | 1141.1179 | 1192.9979 | 145.4212 | 195.4404 | 237.0421 | 171.6771 |
| 13 | 502.0148 | 1053.4171 | 1101.3063 | 134.3125 | 180.4841 | 218.8856 | 158.5486 |

In principle, it is known to the skilled person that slight variations of the migration times may occur in separations by capillary electrophoresis. However, under the conditions described, the order of migration will not change. For the skilled person who knows the stated masses and CE times, it is possible without difficulty to assign their own measurements to the polypeptide markers according to the disclosure. For example, he may proceed as follows: At first, he selects one of the polypeptides found in his measurement (peptide 1) and tries to find one or more identical masses within a time slot of the stated CE time (for example, ±5 min). If only one identical mass is found within this interval, the assignment is completed. If several matching masses are found, a decision about the assignment is still to be made. Thus, another peptide (peptide 2) from the measurement is selected, and it is tried to identify an appropriate polypeptide marker, again taking a corresponding time slot into account.

Again, if several markers can be found with a corresponding mass, the most probable assignment is that in which there is a substantially linear relationship between the shift for peptide 1 and that for peptide 2.

Depending on the complexity of the assignment problem, it suggests itself to the skilled person to optionally use further proteins from his sample for assignment, for example, ten proteins. Typically, the migration times are either extended or shortened by particular absolute values, or compressions or expansions of the whole course occur. However, comigrating peptides will also comigrate under such conditions.

In addition, the skilled person can make use of the migration patterns described by Zuerbig et al. in Electrophoresis 27 (2006), pp. 2111-2125. If he plots his measurement in the form of m/z versus migration time by means of a simple diagram (e.g., with MS Excel), the line patterns described also become visible. Now, a simple assignment of the individual polypeptides is possible by counting the lines.

Other approaches of assignment are also possible. Basically, the skilled person could also use the peptides mentioned above as internal standards for assigning his CE measurements.

What is claimed is:

1. A method of diagnosing vascular diseases (VD) in a subject patient, comprising:
obtaining a urine sample from said subject patient;
purifying said urine sample to remove high concentration proteins;
separating said purified urine sample into a plurality of polypeptides;
identifying said plurality of purified polypeptides based on the separation characteristics of each of said plurality of purified polypeptides in the separating step;
comparing said plurality of purified polypeptides to known polypeptide markers taken from control subjects with VD and without VD to obtain a subset of polypeptide markers from said plurality of purified polypeptides that substantially match said known polypeptide markers, wherein said known polypeptide markers are characterized by the following molecular masses and migration times (CE time):

| No. | Mass | CE time |
|---|---|---|
| 1 | 1166.61 | 23.88 |
| 2 | 2431.50 | 24.10 |
| 3 | 1922.93 | 31.99 |
| 4 | 2509.16 | 25.76 |
| 5 | 3194.22 | 30.34 |
| 6 | 1705.80 | 40.47 |
| 7 | 1962.95 | 31.77 |
| 8 | 3822.12 | 24.72 |
| 9 | 2212.32 | 24.94 |
| 10 | 3015.78 | 35.86 |
| 11 | 1784.95 | 20.94 |
| 12 | 1902.92 | 31.87 |
| 13 | 2329.15 | 27.17 |
| 14 | 2154.05 | 21.78 |
| 15 | 2166.03 | 27.89 |
| 16 | 2258.27 | 21.99 |
| 17 | 2573.84 | 20.49 |
| 18 | 1270.75 | 37.92 |
| 19 | 1611.84 | 40.12 |
| 20 | 1791.87 | 41.04 |
| 21 | 2030.00 | 25.23 |
| 22 | 1290.40 | 30.87 |
| 23 | 1441.74 | 39.13 |
| 24 | 2924.25 | 24.05 |
| 25 | 816.41 | 21.10 |
| 26 | 963.52 | 21.71 |
| 27 | 1503.74 | 29.63 |
| 28 | 2849.59 | 23.02 |
| 29 | 3133.20 | 31.20 |
| 30 | 1283.62 | 27.30 |
| 31 | 1495.75 | 23.31 |
| 32 | 1513.70 | 29.29 |
| 33 | 1612.83 | 23.36 |
| 34 | 2319.19 | 33.80 |
| 35 | 2436.23 | 22.87 |
| 36 | 2557.42 | 28.22 |
| 37 | 2626.85 | 28.00 |
| 38 | 2933.46 | 27.68 |
| 39 | 2994.09 | 29.50 |
| 40 | 4101.34 | 28.51 |
| 41 | 935.49 | 23.69 |
| 42 | 1521.75 | 30.42 |
| 43 | 1669.79 | 21.48 |
| 44 | 2758.37 | 28.94 |
| 45 | 3546.94 | 26.22 |
| 46 | 3609.63 | 20.22 |
| 47 | 3697.49 | 23.71 |
| 48 | 4278.73 | 23.34 |
| 49 | 4421.04 | 20.73 |
| 50 | 4805.67 | 26.49 |
| 51 | 11058.18 | 21.88 |
| 52 | 1352.61 | 29.86 |
| 53 | 2802.85 | 36.35 |
| 54 | 4890.88 | 26.48 |
| 55 | 5212.06 | 26.98 |
| 56 | 945.45 | 25.80 |
| 57 | 1065.55 | 25.50 |
| 58 | 1137.58 | 26.41 |
| 59 | 1542.77 | 23.91 |
| 60 | 1693.83 | 23.47 |
| 61 | 3361.42 | 24.26 |
| 62 | 3617.74 | 26.97 |
| 63 | 3737.69 | 37.15 |
| 64 | 980.54 | 22.44 |
| 65 | 1221.63 | 26.82 |
| 66 | 2952.27 | 25.14 |
| 67 | 3696.88 | 26.94 |
| 68 | 5574.45 | 23.24 |
| 69 | 1182.59 | 28.34 |
| 70 | 1963.96 | 31.76 |
| 71 | 882.54 | 23.81 |
| 72 | 4002.72 | 20.69 |
| 73 | 4059.96 | 20.44 |
| 74 | 1186.59 | 22.31 |
| 75 | 1825.87 | 31.80 |
| 76 | 3401.66 | 23.42 |
| 77 | 1496.75 | 30.36 |
| 78 | 1832.92 | 31.91 |
| 79 | 2281.35 | 36.34 |
| 80 | 2344.34 | 33.66 |
| 81 | 3944.82 | 24.59 |
| 82 | 3002.23 | 23.80 |
| 83 | 3416.77 | 36.76 |
| 84 | 3501.86 | 31.79 |
| 85 | 6783.03 | 26.61 |
| 86 | 14111.27 | 21.97 |
| 87 | 2616.02 | 28.35 |
| 88 | 2810.45 | 36.73 |
| 89 | 2940.95 | 29.07 |
| 90 | 2946.45 | 34.96 |
| 91 | 1494.72 | 30.40 |
| 92 | 1080.53 | 27.86 |
| 93 | 2349.14 | 27.36 |
| 94 | 3303.00 | 23.07 |
| 95 | 4081.56 | 24.51 |
| 96 | 4670.27 | 25.84 |
| 97 | 4671.99 | 23.33 |
| 98 | 8933.94 | 22.57 |
| 99 | 1523.90 | 29.72 |
| 100 | 3956.82 | 25.20 |
| 101 | 3984.81 | 21.29 |
| 102 | 4830.78 | 26.61 |
| 103 | 3031.39 | 35.93 |
| 104 | 3788.76 | 25.21 |
| 105 | 2567.2 | 34.76 |
| 106 | 1447.8 | 19.49 |
| 107 | 2241.51 | 24.11 |
| 108 | 2461.11 | 30.84 |
| 109 | 1965.96 | 23.62 |
| 110 | 2189.08 | 27.17 |
| 111 | 1127.58 | 20.82 |
| 112 | 1400.71 | 20.35 |
| 113 | 1512.75 | 39.51 |
| 114 | 1860.53 | 34.24 |
| 115 | 1442.69 | 27.72 |
| 116 | 2590.78 | 27.96 |
| 117 | 1556.72 | 27.90 |
| 118 | 2309.15 | 21.95 |
| 119 | 2389.33 | 22.34 |
| 120 | 1478.68 | 39.28 |
| 121 | 1795.90 | 24.66 |
| 122 | 2211.03 | 35.06 |
| 123 | 1223.63 | 19.52 |
| 124 | 1829.04 | 21.22 |
| 125 | 1878.66 | 30.19 |
| 126 | 2009.96 | 32.27 |
| 127 | 2110.00 | 24.10 |
| 128 | 1552.79 | 29.75 |
| 129 | 1577.75 | 40.03 |
| 130 | 1936.94 | 34.71 |
| 131 | 2368.13 | 26.75 |
| 132 | 3633.05 | 33.25 |
| 133 | 1510.72 | 28.30 |
| 134 | 1668.87 | 40.49 |
| 135 | 2227.05 | 33.43 |
| 136 | 1495.75 | 39.41 |
| 137 | 1631.77 | 45.38 |
| 138 | 3158.60 | 29.62 |
| 139 | 1522.78 | 22.76 |
| 140 | 1727.87 | 39.61 |
| 141 | 1883.94 | 40.14 |
| 142 | 1460.71 | 19.83 |
| 143 | 1805.88 | 29.92 |
| 144 | 1898.93 | 40.30 |
| 145 | 2237.06 | 27.12 |
| 146 | 3178.33 | 30.26 |
| 147 | 1844.56 | 34.28 |
| 148 | 1378.57 | 37.16 |
| 149 | 1764.86 | 29.88 |
| 150 | 1791.88 | 30.77 |
| 151 | 2082.01 | 33.67 |
| 152 | 1768.90 | 20.77 |
| 153 | 3442.09 | 33.32 |
| 154 | 876.42 | 35.07 |
| 155 | 2352.14 | 26.74 |
| 156 | 937.50 | 34.12 |
| 157 | 1445.72 | 28.36 |
| 158 | 1893.10 | 28.85 |
| 159 | 2839.43 | 24.14 |
| 160 | 1600.76 | 29.61 |
| 161 | 1565.75 | 26.35 |
| 162 | 1627.76 | 29.47 |
| 163 | 1812.90 | 39.98 |
| 164 | 3137.52 | 30.29 |
| 165 | 1364.67 | 28.65 |
| 166 | 2298.07 | 33.82 |
| 167 | 3017.74 | 49.66 |
| 168 | 1235.61 | 26.67 |
| 169 | 1741.81 | 30.21 |
| 170 | 1818.90 | 30.93 |
| 171 | 1892.95 | 22.22 |
| 172 | 3280.69 | 22.69 |
| 173 | 2658.34 | 19.50 |
| 174 | 1407.71 | 27.46 |
| 175 | 1622.79 | 26.82 |
| 176 | 1684.78 | 29.64 |
| 177 | 1321.65 | 28.39 |
| 178 | 1350.68 | 27.13 |
| 179 | 1549.76 | 39.52 |
| 180 | 2233.10 | 22.47 |
| 181 | 2679.27 | 23.48 |
| 182 | 1835.79 | 20.02 |
| 183 | 3421.66 | 25.96 |
| 184 | 1708.85 | 30.44 |
| 185 | 1993.96 | 32.16 |
| 186 | 2695.31 | 23.46 |
| 187 | 1204.65 | 21.93 |
| 188 | 1467.86 | 24.38 |
| 189 | 1767.07 | 24.10 |
| 190 | 8176.30 | 19.57 |
| 191 | 1143.56 | 36.97 |
| 192 | 1834.90 | 31.05 |
| 193 | 2025.95 | 32.21 |
| 194 | 3489.70 | 31.45 |
| 195 | 1268.62 | 27.29 |
| 196 | 1659.82 | 29.35 |
| 197 | 2405.59 | 22.16 |
| 198 | 2483.21 | 27.54 |
| 199 | 2599.21 | 28.20 |
| 200 | 4170.01 | 33.51 |
| 201 | 1013.41 | 25.33 |
| 202 | 1016.49 | 25.88 |
| 203 | 1493.74 | 22.06 |
| 204 | 2104.04 | 32.97 |
| 205 | 3718.81 | 32.39 |
| 206 | 4251.98 | 28.66 |
| 207 | 4538.67 | 26.20 |
| 208 | 2067.93 | 20.68 |
| 209 | 2292.11 | 27.26 |
| 210 | 3702.39 | 32.39 |
| 211 | 965.46 | 27.84 |
| 212 | 2186.07 | 25.89 |
| 213 | 2584.29 | 35.08 |
| 214 | 2841.13 | 24.50 |
| 215 | 9866.78 | 20.85 |
| 216 | 1099.53 | 28.33 |
| 217 | 2471.25 | 34.69 |
| 218 | 3734.85 | 32.41 |
| 219 | 3927.86 | 33.50 |
| 220 | 3166.32 | 22.10 |
| 221 | 2339.08 | 33.95 |
| 222 | 2563.76 | 22.05 |
| 223 | 3219.35 | 35.00 |
| 224 | 3359.66 | 31.84 |
| 225 | 4097.98 | 24.59 |
| 226 | 4654.14 | 25.81 |
| 227 | 1584.77 | 29.72 |
| 228 | 2148.10 | 25.54 |
| 229 | 2639.45 | 21.33 |
| 230 | 3013.27 | 22.27 |
| 231 | 3205.39 | 19.71 |
| 232 | 3831.86 | 28.39 |
| 233 | 1050.52 | 27.03 |
| 234 | 2157.06 | 22.19 |
| 235 | 2407.16 | 27.65 |
| 236 | 2837.93 | 23.99 |
| 237 | 3058.02 | 30.20 |
| 238 | 1658.67 | 21.53 |
| 239 | 3311.32 | 24.46 |
| 240 | 3556.63 | 23.64 |
| 241 | 1085.50 | 21.70 |
| 242 | 1199.63 | 21.91 |
| 243 | 1247.58 | 22.00 |
| 244 | 1608.76 | 22.36 |
| 245 | 2501.20 | 34.30 |
| 246 | 3021.52 | 23.52 |
| 247 | 4153.75 | 33.41 |
| 248 | 5000.17 | 24.43 |
| 249 | 8917.48 | 22.53 |
| 250 | 1750.86 | 23.80 |
| 251 | 2235.13 | 34.10 |
| 252 | 2644.25 | 21.13 |
| 253 | 3943.96 | 33.53 |
| 254 | 11967.96 | 20.50 |
| 255 | 2553.23 | 34.14 |
| 256 | 1209.58 | 26.31 |
| 257 | 1899.94 | 21.41 |
| 258 | 1680.00 | 23.77 |
| 259 | 2195.06 | 20.15 |
| 260 | 3064.41 | 20.55 |
| 261 | 3554.07 | 31.11 |
| 262 | 3686.03 | 22.16 |
| 263 | 3802.12 | 33.10 |
| 264 | 4048.05 | 25.42 |
| 265 | 2380.16 | 36.48 |
| 266 | 1352.83 | 24.38 |
| 267 | 1638.80 | 20.26 |
| 268 | 2864.18 | 20.19 |
| 269 | 3754.66 | 37.16 |
| 270 | 4185.91 | 33.47 |
| 271 | 858.42 | 23.26 |
| 272 | 1159.64 | 26.05 |
| 273 | 1407.71 | 37.25 |
| 274 | 1439.72 | 29.62 |
| 275 | 1720.76 | 19.72 |
| 276 | 1846.93 | 32.04 |
| 277 | 3177.14 | 22.48 |
| 278 | 4113.80 | 24.58 |
| 279 | 2744.07 | 35.03 |
| 280 | 2767.26 | 21.52 |
| 281 | 1310.64 | 27.11 |
| 282 | 1613.88 | 23.95 |
| 283 | 1703.90 | 33.64 |
| 284 | 2761.40 | 21.46 |
| 285 | 3242.42 | 22.78 |
| 286 | 3338.17 | 23.36 |
| 287 | 3371.74 | 22.96 |
| 288 | 3593.53 | 20.25 |
| 289 | 3677.52 | 24.49 |
| 290 | 1624.80 | 30.81 |
| 291 | 2210.92 | 37.55 |
| 292 | 3290.37 | 24.12 |
| 293 | 4413.76 | 29.03 |
| 294 | 1482.73 | 22.47 |
| 295 | 1813.78 | 31.87 |
| 296 | 1934.87 | 20.04 |
| 297 | 2249.89 | 34.14 |
| 298 | 3280.59 | 25.76 |
| 299 | 1098.56 | 21.46 |
| 300 | 1125.58 | 21.76 |
| 301 | 1649.79 | 19.59 |
| 302 | 4025.68 | 20.73 |
| 303 | 980.33 | 35.59 |
| 304 | 1096.41 | 35.95 |
| 305 | 1698.65 | 37.60 |
| 306 | 2361.21 | 20.77 |
| 307 | 3148.50 | 24.22 |
| 308 | 3157.23 | 34.74 |
| 309 | 1304.59 | 27.95 |
| 310 | 3575.78 | 32.27 |
| 311 | 1510.75 | 20.12 |
| 312 | 2485.20 | 34.25 |
| 313 | 3076.33 | 19.64 |
| 314 | 3343.39 | 31.80 |
| 315 | 1405.71 | 20.16 |
| 316 | 2587.16 | 21.07 |
| 317 | 5213.25 | 22.47 |
| 318 | 2320.16 | 20.73 |
| 319 | 4491.89 | 26.23 |
| 320 | 10199.91 | 21.11 |
| 321 | 854.38 | 34.92 |
| 322 | 1084.56 | 36.85 |
| 323 | 1814.78 | 37.29 |
| 324 | 2078.05 | 22.47 |
| 325 | 2175.08 | 33.26 |
| 326 | 2411.78 | 26.97 |
| 327 | 3738.59 | 24.76 |
| 328 | 3935.57 | 34.15 |
| 329 | 4863.21 | 26.66 |
| 330 | 860.39 | 26.25 |
| 331 | 1567.78 | 20.23 |
| 332 | 2308.11 | 27.32 |
| 333 | 2923.77 | 36.44 |
| 334 | 3295.55 | 25.40 |
| 335 | 3870.85 | 33.39 |
| 336 | 1099.56 | 21.63 |
| 337 | 1359.70 | 22.92 |
| 338 | 2059.02 | 23.12 |
| 339 | 2077.03 | 21.78 |
| 340 | 3349.34 | 35.81 |
| 341 | 8853.85 | 21.08 |
| 342 | 1734.80 | 20.24 |
| 343 | 1847.95 | 43.93 |
| 344 | 2045.95 | 34.04 |
| 345 | 2289.47 | 33.56 |
| 346 | 2421.15 | 34.74 |
| 347 | 2480.67 | 23.00 |
| 348 | 2576.25 | 34.17 |
| 349 | 3353.93 | 23.53 |
| 350 | 1083.52 | 26.24 |
| 351 | 2073.17 | 27.43 |
| 352 | 7958.65 | 34.32 |
| 353 | 1837.88 | 30.54 |
| 354 | 2939.03 | 33.75 |
| 355 | 2977.31 | 19.59 |
| 356 | 3596.46 | 21.54 |
| 357 | 3851.68 | 24.97 |
| 358 | 1135.52 | 27.83 |
| 359 | 3378.05 | 38.81 |
| 360 | 3590.72 | 28.99 |
| 361 | 3959.80 | 19.95 |
| 362 | 1258.41 | 36.10 |
| 363 | 1513.50 | 36.82 |
| 364 | 1716.38 | 20.59 |
| 365 | 2022.97 | 33.38 |
| 366 | 2914.54 | 24.29 |
| 367 | 5527.56 | 27.58 |
| 368 | 931.51 | 20.00 |
| 369 | 973.26 | 35.59 |
| 370 | 1385.67 | 27.92 |
| 371 | 2272.31 | 23.80 |
| 372 | 4024.87 | 33.20 |
| 373 | 2216.11 | 33.79 |
| 374 | 2756.23 | 35.16 |
| 375 | 2777.71 | 21.55 |
| 376 | 3521.02 | 30.73 |
| 377 | 3750.72 | 32.45 |
| 378 | 4229.09 | 29.08 |
| 379 | 4846.50 | 26.40 |
| 380 | 1046.55 | 25.35 |
| 381 | 1608.80 | 30.94 |
| 382 | 1878.78 | 31.58 |
| 383 | 2589.16 | 22.45 |
| 384 | 4369.06 | 20.25 |
| 385 | 12717.08 | 26.92 |
| 386 | 1210.43 | 36.48 |
| 387 | 3092.54 | 36.22 |
| 388 | 3248.61 | 25.65 |
| 389 | 4012.41 | 20.81 |
| 390 | 11016.34 | 21.31 |
| 391 | 1284.61 | 29.17 |
| 392 | 1460.83 | 22.53 |
| 393 | 1807.88 | 23.98 |
| 394 | 2596.33 | 34.86 |
| 395 | 2686.97 | 29.06 |
| 396 | 3871.59 | 27.51 |
| 397 | 4069.63 | 25.30 |
| 398 | 4288.98 | 25.94 |
| 399 | 4426.21 | 20.09 |
| 400 | 1071.55 | 21.41 |
| 401 | 1749.88 | 30.54 |
| 402 | 1956.97 | 21.44 |
| 403 | 2189.12 | 26.54 |
| 404 | 2257.63 | 36.10 |
| 405 | 2917.54 | 28.99 |
| 406 | 3633.69 | 26.99 |
| 407 | 6055.77 | 21.03 |
| 408 | 6186.02 | 24.99 |
| 409 | 1858.92 | 24.17 |
| 410 | 2274.11 | 33.47 |
| 411 | 4522.51 | 26.20 |
| 412 | 6237.35 | 31.39 |
| 413 | 9883.82 | 20.84 |
| 414 | 3385.6 | 25.47 |
| 415 | 3745.6 | 26.65 |
| 416 | 1408.7 | 39.13 |
| 417 | 2551.3 | 34.75 |
| 418 | 3265.3 | 36.02 |
| 419 | 2739.3 | 28.4 |
| 420 | 2065 | 24.48 |
| 421 | 2264.1 | 22.67 |
| 422 | 1058.5 | 24.94 |
| 423 | 4467.9 | 29.05 |
| 424 | 2887.4 | 35.66 |
| 425 | 1635.8 | 40.33 |
| 426 | 2525.2 | 27.72 |
| 427 | 1526.8 | 23.63 |
| 428 | 1664.8 | 29.87 |
| 429 | 2583.3 | 28.31 |
| 430 | 2663.3 | 23.44 |
| 431 | 1878.9 | 42.18 |
| 432 | 1462.7 | 39.31 |
| 433 | 1834.9 | 24 |
| 434 | 1893.1 | 24.64 |
| 435 | 1934 | 21.63 |
| 436 | 1367.7 | 38.87 |
| 437 | 1009.5 | 27.33 |
| 438 | 3405.1 | 25.92 |
| 439 | 2314.1 | 33.67 |
| 440 | 3996.8 | 20.93 |
| 441 | 2823.6 | 29.07 |
| 442 | 1179.6 | 27.15 |
| 443 | 1435.7 | 28.86 |
| 444 | 2430.7 | 35.39 |
| 445 | 1134.6 | 23.68 |
| 446 | 2014 | 25.18 |
| 447 | 2577.3 | 24.55 |
| 448 | 1194.6 | 26.73 |
| 449 | 1588.8 | 30.2 |
| 450 | 2056 | 25.44 |
| 451 | 2442.16 | 34.11 |
| 452 | 1422.66 | 21.72 |
| 453 | 1623.8 | 24.15 |
| 454 | 1624.61 | 37.73 |
| 455 | 3298.48 | 36.06 |
| 456 | 1016.31 | 35.67 |
| 457 | 1580.94 | 24.31 |
| 458 | 1157.58 | 37.41 |
| 459 | 1250.61 | 27.94 |
| 460 | 1378.67 | 28.85 |
| 461 | 1392.68 | 21.75 |
| 462 | 1409.64 | 22.06 |
| 463 | 1425.65 | 22.34 |
| 464 | 1451.71 | 29.19 |
| 465 | 1576.66 | 26.5 |
| 466 | 1651.85 | 40.6 |
| 467 | 1876.94 | 22.29 |
| 468 | 1911.12 | 24.98 |
| 469 | 2064.01 | 21.95 |
| 470 | 2150.04 | 27.76 |
| 471 | 2751.59 | 29.16 |
| 472 | 4289.94 | 28.69 |
| 473 | 4306.05 | 28.78 |
| 474 | 4800.18 | 23.83 |
| 475 | 1111.32 | 35.47 |
| 476 | 1181.49 | 36.79 |
| 477 | 3168.38 | 24.69 |
| 478 | 1229.57 | 36.29 |
| 479 | 1579.78 | 29.83 |
| 480 | 1680.82 | 30.02 |
| 481 | 1725.66 | 38.3 |
| 482 | 5228.15 | 27.04 |
| 483 | 1769.78 | 28.25 |
| 484 | 1114.54 | 25.52 |
| 485 | 1390.5 | 37.05 |
| 486 | 2046.99 | 32.56 |
| 487 | 2899.33 | 49.62 |
| 488 | 1096.53 | 26.12 |
| 489 | 1257.49 | 34.26 |
| 490 | 868.45 | 23.35 |
| 491 | 1160.43 | 35.6 |
| 492 | 1539.8 | 40.36 |
| 493 | 3318.91 | 36.01 |
| 494 | 1084.48 | 25.31 |
| 495 | 1388.39 | 58.99 |
| 496 | 3129.86 | 35.93 |
| 497 | 1255.56 | 36.33 |
| 498 | 1383.69 | 39.02 |
| 499 | 1561.75 | 40.72 |
| 500 | 3108.55 | 31.25 |
| 501 | 1173.58 | 37.51 |
| 502 | 1100.55 | 36.99 |
| 503 | 1128.44 | 33.71 |
| 504 | 3149.6 | 31.22 |
| 505 | 1068.56 | 21.69 |
| 506 | 1349.48 | 36.47 |
| 507 | 1689.81 | 40.57 |
| 508 | 2305.7 | 34.8 |
| 509 | 840.44 | 23.94 |
| 510 | 911.3 | 34.39 |
| 511 | 1299.64 | 22.42 |
| 512 | 911.47 | 25.92 |
| 513 | 1025.51 | 25.44 |
| 514 | 3400.07 | 42.03 |
| 515 | 1901.89 | 43.92 |
| 516 | 1110.42 | 34.37 |
| 517 | 1032.5 | 25.89 |
| 518 | 1040.52 | 25.11 |
| 519 | 1265.64 | 27.14 |
| 520 | 1171.55 | 29.24 |
| 521 | 1012.53 | 35.08 |
| 522 | 1286.49 | 36.78 |
| 523 | 2932.36 | 34.11 |
| 524 | 1215.49 | 27.61 |
| 525 | 1423.68 | 21.47 |
| 526 | 1487.71 | 29.58, |

wherein said CE times are based on capillary electrophoresis using a glass capillary of 90 cm in length with an inner diameter (ID) of 50 μm at an applied voltage of 30 kV,
wherein 20% acetonitrile and 0.25% formic acid in water is used as the mobile phase for the capillary electrophoresis at a flow rate of 2 μl/min., and
wherein said CE times are calibrated relative to the following values:

| Protein/Polypeptide | Migration time (minutes) |
|---|---|
| Aprotinin | 9.2 |
| Ribonuclease | 10.9 |
| Lysozyme | 8.9 |
| “REV” SEQ ID NO: 1 | 15.6 |
| “ELM” SEQ ID NO: 2 | 23.4 |
| “KINCON” SEQ ID NO: 3 | 20.0 |
| “GIVLY” SEQ ID NO: 4 | 36.8; |

determining if said subset of polypeptide markers comprises at least a first, second and third biomarker selected from:
a first biomarker that is at least one selected from the group consisting of: (1) markers 1 to 106 (frequency markers) having (a) a frequency of presence in patients with VD of at least 0.4 and (b) a frequency of presence in patients with VD at least three (3) times the frequency of presence in patients without VD; (2) markers 1 to 106 (frequency markers) having (a) a frequency of presence in patients without VD of at least 0.4 and (b) a frequency of presence in patients without VD at least three (3) times the frequency of presence in patients with VD; (3) markers 107 to 526 (amplitude markers) having a mean amplitude in patients with VD of at least 200 and a mean amplitude at least two (2) times greater than the mean amplitude of the same marker in patients without VD; and (4) markers 107 to 526 (amplitude markers) having a mean amplitude in patients without VD of at least 200 and a mean amplitude at least two (2) times greater than the mean amplitude of the same marker in patients with VD;
a second biomarker which is different than said first biomarker and is at least one selected from the group consisting of: (1) markers 1 to 106 (frequency markers) having (a) a frequency of presence in patients with VD of at least 0.4 and (b) a frequency of presence in patients with VD at least three (3) times the frequency of presence in patients without VD; (2) markers 1 to 106 (frequency markers) having (a) a frequency of presence in patients without VD of at least 0.4 and (b) a frequency of presence in patients without VD at least three (3) times the frequency of presence in patients with VD; (3) markers 107 to 526 (amplitude markers) having a mean amplitude in patients with VD of at least 200 and a mean amplitude at least two (2) times greater than the mean amplitude of the same marker in patients without VD; and (4) markers 107 to 526 (amplitude markers) having a mean amplitude in patients without VD of at least 200 and a mean amplitude at least two (2) times greater than the mean amplitude of the same marker in patients with VD; and
a third biomarker which is different than said first and second biomarkers and is at least one selected from the group consisting of markers 1 to 526 (frequency and amplitude markers);
comparing the frequency and/or amplitude of presence of said at least first, second and third biomarkers in said sample of said subject patient to the frequency and/or amplitude of presence of the same at least first, second and third biomarkers from said known polypeptide markers from said control subjects;
ranking said subject patient between said control subjects with VD and without VD based on the second comparing step; and
diagnosing the probability of VD in said subject patient based on said ranking.

2. The method according to claim 1, wherein the frequency of said frequency markers 1 to 106 is compared with the following reference values:

| | Occurrence | Occurrence |
|---|---|---|
| No. | control | CVD |
| 1 | 0.01 | 0.58 |
| 2 | 0.18 | 0.75 |
| 3 | 0.14 | 0.67 |
| 4 | 0.25 | 0.75 |
| 5 | 0.08 | 0.58 |
| 6 | 0.05 | 0.54 |
| 7 | 0.36 | 0.83 |
| 8 | 0.31 | 0.79 |
| 9 | 0.26 | 0.71 |
| 10 | 0.14 | 0.58 |
| 11 | 0.06 | 0.50 |
| 12 | 0.15 | 0.58 |
| 13 | 0.31 | 0.75 |
| 14 | 0.32 | 0.75 |
| 15 | 0.12 | 0.54 |
| 16 | 0.03 | 0.46 |
| 17 | 0.25 | 0.67 |
| 18 | 0.18 | 0.58 |
| 19 | 0.47 | 0.88 |
| 20 | 0.18 | 0.58 |
| 21 | 0.09 | 0.50 |
| 22 | 0.27 | 0.67 |
| 23 | 0.18 | 0.58 |
| 24 | 0.10 | 0.50 |
| 25 | 0.44 | 0.04 |
| 26 | 0.44 | 0.04 |
| 27 | 0.44 | 0.04 |
| 28 | 0.48 | 0.08 |
| 29 | 0.74 | 0.33 |
| 30 | 0.62 | 0.21 |
| 31 | 0.45 | 0.04 |
| 32 | 0.41 | 0.00 |
| 33 | 0.62 | 0.21 |
| 34 | 0.74 | 0.33 |
| 35 | 0.41 | 0.00 |
| 36 | 0.78 | 0.38 |
| 37 | 0.45 | 0.04 |
| 38 | 0.70 | 0.29 |
| 39 | 0.78 | 0.38 |
| 40 | 0.41 | 0.00 |
| 41 | 0.67 | 0.25 |
| 42 | 0.59 | 0.17 |
| 43 | 0.67 | 0.25 |
| 44 | 0.54 | 0.13 |
| 45 | 0.63 | 0.21 |
| 46 | 0.42 | 0.00 |
| 47 | 0.80 | 0.38 |
| 48 | 0.54 | 0.13 |
| 49 | 0.42 | 0.00 |
| 50 | 0.58 | 0.17 |
| 51 | 0.42 | 0.00 |
| 52 | 0.72 | 0.29 |
| 53 | 0.52 | 0.08 |
| 54 | 0.52 | 0.08 |
| 55 | 0.59 | 0.17 |
| 56 | 0.65 | 0.21 |
| 57 | 0.48 | 0.04 |
| 58 | 0.57 | 0.13 |
| 59 | 0.56 | 0.13 |
| 60 | 0.44 | 0.00 |
| 61 | 0.57 | 0.13 |
| 62 | 0.44 | 0.00 |
| 63 | 0.73 | 0.29 |
| 64 | 0.53 | 0.08 |
| 65 | 0.50 | 0.04 |
| 66 | 0.49 | 0.04 |
| 67 | 0.70 | 0.25 |
| 68 | 0.91 | 0.46 |
| 69 | 0.71 | 0.25 |
| 70 | 0.63 | 0.17 |
| 71 | 0.47 | 0.00 |
| 72 | 0.81 | 0.33 |
| 73 | 0.59 | 0.13 |
| 74 | 0.90 | 0.42 |
| 75 | 0.77 | 0.29 |
| 76 | 0.94 | 0.46 |
| 77 | 0.69 | 0.21 |
| 78 | 0.49 | 0.00 |
| 79 | 0.66 | 0.17 |
| 80 | 0.53 | 0.04 |
| 81 | 0.53 | 0.04 |
| 82 | 0.67 | 0.17 |
| 83 | 0.50 | 0.00 |
| 84 | 0.50 | 0.00 |
| 85 | 0.62 | 0.13 |
| 86 | 0.88 | 0.38 |
| 87 | 0.63 | 0.13 |
| 88 | 0.55 | 0.04 |
| 89 | 0.52 | 0.00 |
| 90 | 0.61 | 0.08 |
| 91 | 0.91 | 0.38 |
| 92 | 0.75 | 0.21 |
| 93 | 0.67 | 0.13 |
| 94 | 0.73 | 0.17 |
| 95 | 0.60 | 0.04 |
| 96 | 0.69 | 0.13 |
| 97 | 0.85 | 0.29 |
| 98 | 0.60 | 0.04 |
| 99 | 0.70 | 0.13 |
| 100 | 0.71 | 0.13 |
| 101 | 0.58 | 0.00 |
| 102 | 0.59 | 0.00 |
| 103 | 0.80 | 0.17 |
| 104 | 0.77 | 0.08 |
| 105 | 0.98 | 0.49 |
| 106 | 0.90 | 0.49 |

wherein the amplitude of said amplitude markers 107 to 413 is compared with the following reference values:

| | Mean | |
|---|---|---|
| | amplitude | Mean |
| | control | amplitude |
| No. | group | CVD group |
| 107 | 94 | 253 |
| 108 | 116 | 233 |
| 109 | 50 | 123 |
| 110 | 766 | 1878 |
| 111 | 45 | 175 |
| 112 | 89 | 419 |
| 113 | 69 | 146 |
| 114 | 174 | 418 |
| 115 | 78 | 689 |
| 116 | 47 | 99 |
| 117 | 59 | 188 |
| 118 | 120 | 357 |
| 119 | 317 | 2460 |
| 120 | 121 | 463 |
| 121 | 172 | 380 |
| 122 | 796 | 1674 |
| 123 | 167 | 888 |
| 124 | 1703 | 636 |
| 125 | 768 | 3651 |
| 126 | 340 | 1283 |
| 127 | 193 | 583 |
| 128 | 135 | 320 |
| 129 | 243 | 566 |
| 130 | 95 | 214 |
| 131 | 161 | 768 |
| 132 | 118 | 299 |
| 133 | 116 | 267 |
| 134 | 840 | 1950 |
| 135 | 102 | 288 |
| 136 | 127 | 283 |
| 137 | 5726 | 12044 |
| 138 | 263 | 728 |
| 139 | 506 | 154 |
| 140 | 113 | 289 |
| 141 | 150 | 301 |
| 142 | 136 | 290 |
| 143 | 51 | 189 |
| 144 | 168 | 343 |
| 145 | 196 | 769 |
| 146 | 119 | 250 |
| 147 | 161 | 358 |
| 148 | 227 | 58 |
| 149 | 130 | 289 |
| 150 | 97 | 196 |
| 151 | 192 | 504 |
| 152 | 301 | 128 |
| 153 | 442 | 108 |
| 154 | 154 | 1119 |
| 155 | 197 | 725 |
| 156 | 82 | 201 |
| 157 | 100 | 202 |
| 158 | 189 | 513 |
| 159 | 1054 | 486 |
| 160 | 161 | 412 |
| 161 | 123 | 456 |
| 162 | 229 | 517 |
| 163 | 273 | 554 |
| 164 | 196 | 517 |
| 165 | 197 | 97 |
| 166 | 176 | 506 |
| 167 | 1480 | 686 |
| 168 | 3107 | 880 |
| 169 | 80 | 216 |
| 170 | 203 | 1328 |
| 171 | 344 | 848 |
| 172 | 797 | 206 |
| 173 | 1146 | 2842 |
| 174 | 224 | 568 |
| 175 | 138 | 450 |
| 176 | 258 | 525 |
| 177 | 15571 | 7296 |
| 178 | 367 | 745 |
| 179 | 185 | 575 |
| 180 | 260 | 130 |
| 181 | 1492 | 3433 |
| 182 | 784 | 351 |
| 183 | 1158 | 2826 |
| 184 | 919 | 371 |
| 185 | 156 | 466 |
| 186 | 1694 | 4348 |
| 187 | 201 | 66 |
| 188 | 1787 | 737 |
| 189 | 2810 | 6060 |
| 190 | 1703 | 766 |
| 191 | 461 | 1095 |
| 192 | 707 | 6865 |
| 193 | 493 | 1490 |
| 194 | 346 | 799 |
| 195 | 3338 | 9120 |
| 196 | 240 | 654 |
| 197 | 80 | 203 |
| 198 | 490 | 236 |
| 199 | 259 | 533 |
| 200 | 1142 | 421 |
| 201 | 1241 | 2506 |
| 202 | 1511 | 749 |
| 203 | 294 | 107 |
| 204 | 1090 | 2230 |
| 205 | 1151 | 456 |
| 206 | 983 | 475 |
| 207 | 220 | 44 |
| 208 | 1195 | 546 |
| 209 | 3909 | 1825 |
| 210 | 406 | 167 |
| 211 | 149 | 58 |
| 212 | 1098 | 2400 |
| 213 | 769 | 164 |
| 214 | 527 | 1675 |
| 215 | 1173 | 416 |
| 216 | 711 | 324 |
| 217 | 470 | 181 |
| 218 | 723 | 333 |
| 219 | 213 | 102 |
| 220 | 345 | 169 |
| 221 | 677 | 334 |
| 222 | 2489 | 744 |
| 223 | 132 | 63 |
| 224 | 1451 | 717 |
| 225 | 1324 | 299 |
| 226 | 1689 | 741 |
| 227 | 88 | 238 |
| 228 | 191 | 701 |
| 229 | 361 | 118 |
| 230 | 5095 | 1789 |
| 231 | 601 | 241 |
| 232 | 1200 | 403 |
| 233 | 736 | 245 |
| 234 | 1171 | 297 |
| 235 | 678 | 263 |
| 236 | 1597 | 482 |
| 237 | 115 | 353 |
| 238 | 392 | 146 |
| 239 | 120 | 265 |
| 240 | 1127 | 465 |
| 241 | 623 | 140 |
| 242 | 250 | 115 |
| 243 | 633 | 306 |
| 244 | 224 | 81 |
| 245 | 120 | 60 |
| 246 | 1275 | 438 |
| 247 | 283 | 89 |
| 248 | 1514 | 737 |
| 249 | 264 | 91 |
| 250 | 900 | 278 |
| 251 | 776 | 338 |
| 252 | 411 | 97 |
| 253 | 227 | 103 |
| 254 | 186 | 53 |
| 255 | 890 | 332 |
| 256 | 469 | 1170 |
| 257 | 152 | 70 |
| 258 | 21200 | 7156 |
| 259 | 282 | 115 |
| 260 | 474 | 152 |
| 261 | 117 | 274 |
| 262 | 1359 | 517 |
| 263 | 421 | 195 |
| 264 | 183 | 83 |
| 265 | 151 | 64 |
| 266 | 206 | 99 |
| 267 | 588 | 256 |
| 268 | 304 | 119 |
| 269 | 147 | 61 |
| 270 | 172 | 66 |
| 271 | 338 | 157 |
| 272 | 292 | 138 |
| 273 | 227 | 110 |
| 274 | 142 | 387 |
| 275 | 166 | 79 |
| 276 | 179 | 385 |
| 277 | 200 | 75 |
| 278 | 211 | 81 |
| 279 | 169 | 68 |
| 280 | 359 | 157 |
| 281 | 141 | 284 |
| 282 | 244 | 104 |
| 283 | 882 | 331 |
| 284 | 903 | 324 |
| 285 | 231 | 98 |
| 286 | 1420 | 457 |
| 287 | 2096 | 591 |
| 288 | 676 | 261 |
| 289 | 470 | 234 |
| 290 | 169 | 49 |
| 291 | 234 | 517 |
| 292 | 624 | 309 |
| 293 | 279 | 111 |
| 294 | 444 | 130 |
| 295 | 752 | 1640 |
| 296 | 543 | 191 |
| 297 | 164 | 66 |
| 298 | 785 | 274 |
| 299 | 185 | 79 |
| 300 | 234 | 99 |
| 301 | 179 | 46 |
| 302 | 360 | 141 |
| 303 | 106 | 37 |
| 304 | 146 | 47 |
| 305 | 730 | 323 |
| 306 | 373 | 40 |
| 307 | 97 | 39 |
| 308 | 48 | 104 |
| 309 | 176 | 375 |
| 310 | 87 | 185 |
| 311 | 187 | 91 |
| 312 | 143 | 43 |
| 313 | 381 | 143 |
| 314 | 402 | 194 |
| 315 | 237 | 113 |
| 316 | 519 | 207 |
| 317 | 115 | 56 |
| 318 | 197 | 61 |
| 319 | 254 | 1335 |
| 320 | 283 | 140 |
| 321 | 88 | 201 |
| 322 | 119 | 56 |
| 323 | 129 | 46 |
| 324 | 125 | 50 |
| 325 | 3240 | 7677 |
| 326 | 114 | 51 |
| 327 | 236 | 89 |
| 328 | 163 | 79 |
| 329 | 702 | 204 |
| 330 | 481 | 159 |
| 331 | 407 | 175 |
| 332 | 228 | 79 |
| 333 | 200 | 98 |
| 334 | 356 | 80 |
| 335 | 152 | 72 |
| 336 | 178 | 64 |
| 337 | 281 | 50 |
| 338 | 293 | 104 |
| 339 | 796 | 299 |
| 340 | 174 | 83 |
| 341 | 1025 | 194 |
| 342 | 209 | 95 |
| 343 | 407 | 145 |
| 344 | 144 | 462 |
| 345 | 182 | 74 |
| 346 | 95 | 42 |
| 347 | 92 | 16 |
| 348 | 150 | 36 |
| 349 | 256 | 96 |
| 350 | 130 | 51 |
| 351 | 96 | 46 |
| 352 | 330 | 157 |
| 353 | 248 | 107 |
| 354 | 205 | 69 |
| 355 | 310 | 36 |
| 356 | 411 | 139 |
| 357 | 789 | 179 |
| 358 | 263 | 104 |
| 359 | 184 | 84 |
| 360 | 206 | 52 |
| 361 | 755 | 287 |
| 362 | 246 | 26 |
| 363 | 316 | 106 |
| 364 | 1329 | 142 |
| 365 | 122 | 23 |
| 366 | 105 | 46 |
| 367 | 311 | 127 |
| 368 | 131 | 56 |
| 369 | 206 | 38 |
| 370 | 104 | 41 |
| 371 | 126 | 43 |
| 372 | 345 | 110 |
| 373 | 416 | 151 |
| 374 | 209 | 86 |
| 375 | 268 | 54 |
| 375 | 549 | 188 |
| 377 | 115 | 36 |
| 378 | 353 | 110 |
| 379 | 379 | 135 |
| 380 | 503 | 52 |
| 381 | 753 | 360 |
| 382 | 335 | 2617 |
| 383 | 97 | 31 |
| 384 | 1280 | 178 |
| 385 | 438 | 64 |
| 386 | 374 | 92 |
| 387 | 329 | 109 |
| 388 | 283 | 124 |
| 389 | 273 | 36 |
| 390 | 3045 | 864 |
| 391 | 51 | 25 |
| 392 | 711 | 69 |
| 393 | 187 | 82 |
| 394 | 74 | 29 |
| 395 | 197 | 61 |
| 396 | 320 | 100 |
| 397 | 712 | 156 |
| 398 | 187 | 48 |
| 399 | 337 | 7 |
| 400 | 133 | 59 |
| 401 | 297 | 110 |
| 402 | 164 | 55 |
| 403 | 876 | 4574 |
| 404 | 820 | 309 |
| 405 | 845 | 288 |
| 406 | 475 | 119 |
| 407 | 275 | 91 |
| 403 | 1590 | 291 |
| 409 | 1343 | 334 |
| 410 | 180 | 61 |
| 411 | 149 | 13 |
| 412 | 298 | 135 |
| 413 | 469 | 42 |

and wherein the amplitude of said amplitude markers 414 to 526 is compared with the following reference values:

| | Mean amplitude | Mean amplitude |
|---|---|---|
| No. | control group | CVD group |
| 414 | 3214 | 2678 |
| 415 | 514 | 250 |
| 416 | 1359 | 615 |
| 417 | 581 | 174 |
| 418 | 630 | 499 |
| 419 | 212 | 141 |
| 420 | 681 | 381 |
| 421 | 445 | 227 |
| 422 | 1178 | 103 |
| 423 | 540 | 348 |
| 424 | 188 | 206 |
| 425 | 1540 | 687 |
| 426 | 569 | 914 |
| 427 | 301 | 106 |
| 428 | 976 | 511 |
| 429 | 972 | 515 |
| 430 | 1320 | 729 |
| 431 | 278 | 210 |
| 432 | 1682 | 1196 |
| 433 | 589 | 287 |
| 434 | 384 | 502 |
| 435 | 1006 | 399 |
| 436 | 1064 | 800 |
| 437 | 270 | 216 |
| 438 | 3453 | 2235 |
| 439 | 837 | 790 |
| 440 | 1353 | 684 |
| 441 | 710 | 733 |
| 442 | 809 | 627 |
| 443 | 8328 | 3904 |
| 444 | 596 | 661 |
| 445 | 380 | 593 |
| 446 | 2389 | 1375 |
| 447 | 297 | 285 |
| 448 | 4154 | 2314 |
| 449 | 532 | 953 |
| 450 | 1145 | 733 |
| 451 | 744 | 845 |
| 452 | 2878 | 2433 |
| 453 | 8725 | 4307 |
| 454 | 1109 | 1620 |
| 455 | 750 | 409 |
| 456 | 687 | 971 |
| 457 | 2155 | 1229 |
| 458 | 1622 | 1964 |
| 459 | 50512 | 45582 |
| 460 | 2259 | 2915 |
| 461 | 4142 | 4664 |
| 462 | 6265 | 8932 |
| 463 | 1969 | 2931 |
| 464 | 36818 | 19376 |
| 465 | 1352 | 1591 |
| 466 | 5789 | 1562 |
| 467 | 2562 | 1358 |
| 468 | 91735 | 90455 |
| 469 | 7723 | 4053 |
| 470 | 2496 | 1342 |
| 471 | 358 | 423 |
| 472 | 3360 | 3317 |
| 473 | 4000 | 2575 |
| 474 | 1388 | 2785 |
| 475 | 335 | 3122 |
| 476 | 302 | 237 |
| 477 | 351 | 847 |
| 478 | 186 | 145 |
| 479 | 3094 | 2397 |
| 480 | 4737 | 2446 |
| 481 | 1468 | 2644 |
| 482 | 566 | 974 |
| 483 | 535 | 429 |
| 484 | 2818 | 4530 |
| 485 | 17423 | 37226 |
| 486 | 3087 | 1793 |
| 487 | 25 | 319 |
| 488 | 3397 | 6633 |
| 489 | 2904 | 6138 |
| 490 | 239 | 198 |
| 491 | 1794 | 3083 |
| 492 | 2558 | 1701 |
| 493 | 428 | 419 |
| 494 | 1326 | 2891 |
| 495 | 181 | 788 |
| 496 | 212 | 207 |
| 497 | 741 | 600 |
| 498 | 135 | 197 |
| 499 | 4632 | 4647 |
| 500 | 331 | 461 |
| 501 | 302 | 414 |
| 502 | 206 | 306 |
| 503 | 1521 | 3346 |
| 504 | 349 | 561 |
| 505 | 211 | 315 |
| 506 | 208 | 247 |
| 507 | 1270 | 1039 |
| 508 | 305 | 334 |
| 509 | 213 | 266 |
| 510 | 2436 | 3827 |
| 511 | 460 | 294 |
| 512 | 389 | 924 |
| 513 | 197 | 273 |
| 514 | 152 | 448 |
| 515 | 743 | 575 |
| 516 | 428 | 713 |
| 517 | 186 | 298 |
| 518 | 219 | 296 |
| 519 | 4218 | 7618 |
| 520 | 75 | 148 |
| 521 | 96 | 214 |
| 522 | 56 | 92 |
| 523 | 208 | 316 |
| 524 | 349 | 729 |
| 525 | 543 | 1220 |
| 526 | 388 | 684 |

3. The method according to claim 1, wherein at least five (5) polypeptide markers are used.

4. The method according to claim 1, wherein said capillary electrophoresis is performed before said molecular mass of said polypeptide markers is measured.

5. The method according to claim 1, wherein said mass spectrometry is also used for detecting the presence or absence of said polypeptide markers.

6. The method according to claim 1, wherein at least ten polypeptide markers are used.

7. The method according to claim 1, wherein all 526 polypeptide markers are used.

8. The method according to claim 1, wherein polypeptide markers 1 to 104 are used.

9. The method according to claim 1, wherein polypeptide markers 107 to 413 are used.

10. The method according to claim 1, further comprising identifying the amino acid sequence of said at least three polypeptide markers.

11. A method of diagnosing vascular diseases (VD) in a subject patient, comprising:
obtaining a urine sample from said subject;
pretreating said urine sample by a method selected from the group consisting of purification, separation, dilution, concentration or any combinations of the foregoing;
purifying said pretreated urine sample to remove high concentration proteins;
separating said purified urine sample into a plurality of polypeptides;
identifying said plurality of pretreated and purified polypeptides based on the separation characteristics of each of said plurality of pretreated and purified polypeptides in the separating step;
comparing said plurality of pretreated and purified polypeptides to known polypeptide markers taken from control subjects with VD and without VD to obtain a subset of polypeptide markers from said plurality of pretreated and purified polypeptides that substantially match said known polypeptide markers, wherein said known polypeptide markers are characterized by the following molecular masses and migration times (CE time):

| No. | Mass | CE time |
|---|---|---|
| 1 | 1166.61 | 23.88 |
| 2 | 2431.50 | 24.10 |
| 3 | 1922.93 | 31.99 |
| 4 | 2509.16 | 25.76 |
| 5 | 3194.22 | 30.34 |
| 6 | 1705.80 | 40.47 |
| 7 | 1962.95 | 31.77 |
| 8 | 3822.12 | 24.72 |
| 9 | 2212.32 | 24.94 |
| 10 | 3015.78 | 35.86 |
| 11 | 1784.95 | 20.94 |
| 12 | 1902.92 | 31.87 |
| 13 | 2329.15 | 27.17 |
| 14 | 2154.05 | 21.78 |
| 15 | 2166.03 | 27.89 |
| 16 | 2258.27 | 21.99 |
| 17 | 2573.84 | 20.49 |
| 18 | 1270.75 | 37.92 |
| 19 | 1611.84 | 40.12 |
| 20 | 1791.87 | 41.04 |
| 21 | 2030.00 | 25.23 |
| 22 | 1290.40 | 30.87 |
| 23 | 1441.74 | 39.13 |
| 24 | 2924.25 | 24.05 |
| 25 | 816.41 | 21.10 |
| 26 | 963.52 | 21.71 |
| 27 | 1503.74 | 29.63 |
| 28 | 2849.59 | 23.02 |
| 29 | 3133.20 | 31.20 |
| 30 | 1283.62 | 27.30 |
| 31 | 1495.75 | 23.31 |
| 32 | 1513.70 | 29.29 |
| 33 | 1612.83 | 23.36 |
| 34 | 2319.19 | 33.80 |
| 35 | 2436.23 | 22.87 |
| 36 | 2557.42 | 28.22 |
| 37 | 2626.85 | 28.00 |
| 38 | 2933.46 | 27.68 |
| 39 | 2994.09 | 29.50 |
| 40 | 4101.34 | 28.51 |
| 41 | 935.49 | 23.69 |
| 42 | 1521.75 | 30.42 |
| 43 | 1669.79 | 21.48 |
| 44 | 2758.37 | 28.94 |
| 45 | 3546.94 | 26.22 |
| 46 | 3609.63 | 20.22 |
| 47 | 3697.49 | 23.71 |
| 48 | 4278.73 | 23.34 |
| 49 | 4421.04 | 20.73 |
| 50 | 4805.67 | 26.49 |
| 51 | 11058.18 | 21.88 |
| 52 | 1352.61 | 29.86 |
| 53 | 2802.85 | 36.35 |
| 54 | 4890.88 | 26.48 |
| 55 | 5212.06 | 26.98 |
| 56 | 945.45 | 25.80 |
| 57 | 1065.55 | 25.50 |
| 58 | 1137.58 | 26.41 |
| 59 | 1542.77 | 23.91 |
| 60 | 1693.83 | 23.47 |
| 61 | 3361.42 | 24.26 |
| 62 | 3617.74 | 26.97 |
| 63 | 3737.69 | 37.15 |
| 64 | 980.54 | 22.44 |
| 65 | 1221.63 | 26.82 |
| 66 | 2952.27 | 25.14 |
| 67 | 3696.88 | 26.94 |
| 68 | 5574.45 | 23.24 |
| 69 | 1182.59 | 28.34 |
| 70 | 1963.96 | 31.76 |
| 71 | 882.54 | 23.81 |
| 72 | 4002.72 | 20.69 |
| 73 | 4059.96 | 20.44 |
| 74 | 1186.59 | 22.31 |
| 75 | 1825.87 | 31.80 |
| 76 | 3401.66 | 23.42 |
| 77 | 1496.75 | 30.36 |
| 78 | 1832.92 | 31.91 |
| 79 | 2281.35 | 36.34 |
| 80 | 2344.34 | 33.66 |
| 81 | 3944.82 | 24.59 |
| 82 | 3002.23 | 23.80 |
| 83 | 3416.77 | 36.76 |
| 84 | 3501.86 | 31.79 |
| 85 | 5783.03 | 26.61 |
| 86 | 14111.27 | 21.97 |
| 87 | 2616.02 | 28.35 |
| 88 | 2810.45 | 36.73 |
| 89 | 2940.95 | 29.07 |
| 90 | 2946.45 | 34.96 |
| 91 | 1494.72 | 30.40 |
| 92 | 1080.53 | 27.86 |
| 93 | 2349.14 | 27.36 |
| 94 | 3303.00 | 23.07 |
| 95 | 4081.56 | 24.51 |
| 96 | 4670.27 | 25.84 |
| 97 | 4671.99 | 23.33 |
| 98 | 8933.94 | 22.57 |
| 99 | 1523.90 | 29.72 |
| 100 | 3956.82 | 25.20 |
| 101 | 3984.81 | 21.29 |
| 102 | 4830.78 | 26.61 |
| 103 | 3031.39 | 35.93 |
| 104 | 3788.76 | 25.21 |
| 105 | 2567.2 | 34.76 |
| 106 | 1447.8 | 19.49 |
| 107 | 2241.51 | 24.11 |
| 108 | 2461.11 | 30.84 |
| 109 | 1965.96 | 23.62 |
| 110 | 2189.08 | 27.17 |
| 111 | 1127.58 | 20.82 |
| 112 | 1400.71 | 20.35 |
| 113 | 1512.75 | 39.51 |
| 114 | 1860.53 | 34.24 |
| 115 | 1442.69 | 27.72 |
| 116 | 2590.78 | 27.96 |
| 117 | 1556.72 | 27.90 |
| 118 | 2309.15 | 21.95 |
| 119 | 2389.33 | 22.34 |
| 120 | 1478.68 | 39.28 |
| 121 | 1795.90 | 24.66 |
| 122 | 2211.03 | 35.06 |
| 123 | 1223.63 | 19.52 |
| 124 | 1829.04 | 21.22 |
| 125 | 1878.66 | 30.19 |
| 126 | 2009.96 | 32.27 |
| 127 | 2110.00 | 24.10 |
| 128 | 1552.79 | 29.75 |
| 129 | 1577.75 | 40.03 |
| 130 | 1936.94 | 34.71 |
| 131 | 2368.13 | 26.75 |
| 132 | 3633.05 | 33.25 |
| 133 | 1510.72 | 28.30 |
| 134 | 1668.87 | 40.49 |
| 135 | 2227.05 | 33.43 |
| 136 | 1495.75 | 39.41 |
| 137 | 1631.77 | 45.38 |
| 138 | 3158.60 | 29.62 |
| 139 | 1522.78 | 22.76 |
| 140 | 1727.87 | 39.61 |
| 141 | 1883.94 | 40.14 |
| 142 | 1460.71 | 19.83 |
| 143 | 1805.88 | 29.92 |
| 144 | 1898.93 | 40.30 |
| 145 | 2237.06 | 27.12 |
| 146 | 3178.33 | 30.26 |
| 147 | 1844.56 | 34.28 |
| 148 | 1378.57 | 37.16 |
| 149 | 1764.86 | 29.88 |
| 150 | 1791.88 | 30.77 |
| 151 | 2082.01 | 33.67 |
| 152 | 1768.90 | 20.77 |
| 153 | 3442.09 | 33.32 |
| 154 | 876.42 | 35.07 |
| 155 | 2352.14 | 26.74 |
| 156 | 937.50 | 34.12 |
| 157 | 1445.72 | 28.36 |
| 158 | 1893.10 | 28.85 |
| 159 | 2839.43 | 24.14 |
| 160 | 1600.76 | 29.61 |
| 161 | 1565.75 | 26.35 |
| 162 | 1627.76 | 29.47 |
| 163 | 1812.90 | 39.98 |
| 164 | 3137.52 | 30.29 |
| 165 | 1364.67 | 28.65 |
| 166 | 2298.07 | 33.82 |
| 167 | 3017.74 | 49.66 |
| 168 | 1235.61 | 26.67 |
| 169 | 1741.81 | 30.21 |
| 170 | 1818.90 | 30.93 |
| 171 | 1892.95 | 22.22 |
| 172 | 3280.69 | 22.69 |
| 173 | 2658.34 | 19.50 |
| 174 | 1407.71 | 27.46 |
| 175 | 1622.79 | 26.82 |
| 176 | 1684.78 | 29.64 |
| 177 | 1321.65 | 28.39 |
| 178 | 1350.68 | 27.13 |
| 179 | 1549.76 | 39.52 |
| 180 | 2233.10 | 22.47 |
| 181 | 2679.27 | 23.48 |
| 182 | 1835.79 | 20.02 |
| 183 | 3421.66 | 25.96 |
| 184 | 1708.85 | 30.44 |
| 185 | 1993.96 | 32.16 |
| 186 | 2695.31 | 23.46 |
| 187 | 1204.65 | 21.93 |
| 188 | 1467.86 | 24.38 |
| 189 | 1767.07 | 24.10 |
| 190 | 8176.30 | 19.57 |
| 191 | 1143.56 | 36.97 |
| 192 | 1834.90 | 31.05 |
| 193 | 2025.95 | 32.21 |
| 194 | 3489.70 | 31.45 |
| 195 | 1268.62 | 27.29 |
| 196 | 1659.82 | 29.35 |
| 197 | 2405.59 | 22.16 |
| 198 | 2483.21 | 27.54 |
| 199 | 2599.21 | 28.20 |
| 200 | 4170.01 | 33.51 |
| 201 | 1013.41 | 25.33 |
| 202 | 1016.49 | 25.88 |
| 203 | 1493.74 | 22.06 |
| 204 | 2104.04 | 32.97 |
| 205 | 3718.81 | 32.39 |
| 206 | 4251.98 | 28.66 |
| 207 | 4538.67 | 26.20 |
| 208 | 2067.93 | 20.68 |
| 209 | 2292.11 | 27.26 |
| 210 | 3702.39 | 32.39 |
| 211 | 965.46 | 27.84 |
| 212 | 2186.07 | 25.89 |
| 213 | 2584.29 | 35.08 |
| 214 | 2841.13 | 24.50 |
| 215 | 9866.78 | 20.85 |
| 216 | 1099.53 | 28.33 |
| 217 | 2471.25 | 34.69 |
| 218 | 3734.85 | 32.41 |
| 219 | 3927.86 | 33.50 |
| 220 | 3166.32 | 22.10 |
| 221 | 2339.08 | 33.95 |
| 222 | 2563.76 | 22.05 |
| 223 | 3219.35 | 35.00 |
| 224 | 3359.66 | 31.84 |
| 225 | 4097.98 | 24.59 |
| 226 | 4654.14 | 25.81 |
| 227 | 1584.77 | 29.72 |
| 228 | 2148.10 | 25.54 |
| 229 | 2639.45 | 21.33 |
| 230 | 3013.27 | 22.27 |
| 231 | 3205.39 | 19.71 |
| 232 | 3831.86 | 28.39 |
| 233 | 1050.52 | 27.03 |
| 234 | 2157.06 | 22.19 |
| 235 | 2407.16 | 27.65 |
| 236 | 2837.93 | 23.99 |
| 237 | 3058.02 | 30.20 |
| 238 | 1658.67 | 21.53 |
| 239 | 3311.32 | 24.46 |
| 240 | 3556.63 | 23.64 |
| 241 | 1085.50 | 21.70 |
| 242 | 1199.63 | 21.91 |
| 243 | 1247.58 | 22.00 |
| 244 | 1608.76 | 22.36 |
| 245 | 2501.20 | 34.30 |
| 246 | 3021.52 | 23.52 |
| 247 | 4153.75 | 33.41 |
| 248 | 5000.17 | 24.43 |
| 249 | 8917.48 | 22.53 |
| 250 | 1750.86 | 23.80 |
| 251 | 2235.13 | 34.10 |
| 252 | 2644.25 | 21.13 |
| 253 | 3943.96 | 33.53 |
| 254 | 11967.96 | 20.50 |
| 255 | 2553.23 | 34.14 |
| 256 | 1209.58 | 26.31 |
| 257 | 1899.94 | 121.41 |
| 258 | 1680.00 | 23.77 |
| 259 | 2195.06 | 20.15 |
| 260 | 3064.41 | 20.55 |
| 261 | 3554.07 | 31.11 |
| 262 | 3686.03 | 22.16 |
| 263 | 3802.12 | 33.10 |
| 264 | 4048.05 | 25.42 |
| 265 | 2380.16 | 36.48 |
| 266 | 1352.83 | 24.38 |
| 267 | 1638.80 | 20.26 |
| 268 | 2864.18 | 20.19 |
| 269 | 3754.66 | 37.16 |
| 270 | 4185.91 | 33.47 |
| 271 | 858.42 | 23.26 |
| 272 | 1159.64 | 26.05 |
| 273 | 1407.71 | 37.25 |
| 274 | 1439.72 | 29.62 |
| 275 | 1720.76 | 19.72 |
| 276 | 1846.93 | 32.04 |
| 277 | 3177.14 | 22.48 |
| 278 | 4113.80 | 24.58 |
| 279 | 2744.07 | 35.03 |
| 280 | 2767.26 | 21.52 |
| 281 | 1310.64 | 27.11 |
| 282 | 1613.88 | 23.95 |
| 283 | 1703.90 | 33.64 |
| 284 | 2761.40 | 21.46 |
| 285 | 3242.42 | 22.78 |
| 286 | 3338.17 | 23.36 |
| 287 | 3371.74 | 22.96 |
| 288 | 3593.53 | 20.25 |
| 289 | 3677.52 | 24.49 |
| 290 | 1624.80 | 30.81 |
| 291 | 2210.92 | 37.55 |
| 292 | 3290.37 | 24.12 |
| 293 | 4413.76 | 29.03 |
| 294 | 1482.73 | 22.47 |
| 295 | 1813.78 | 31.87 |
| 296 | 1934.87 | 20.04 |
| 297 | 2249.89 | 34.14 |
| 298 | 3280.59 | 25.76 |
| 299 | 1098.56 | 21.46 |
| 300 | 1125.58 | 21.76 |
| 301 | 1649.79 | 19.59 |
| 302 | 4025.68 | 20.73 |
| 303 | 980.33 | 35.59 |
| 304 | 1096.41 | 35.95 |
| 305 | 1698.65 | 37.60 |
| 306 | 2361.21 | 20.77 |
| 307 | 3148.50 | 24.22 |
| 308 | 3157.23 | 34.74 |
| 309 | 1304.59 | 27.95 |
| 310 | 3575.78 | 32.27 |
| 311 | 1510.75 | 20.12 |
| 312 | 2485.20 | 34.25 |
| 313 | 3076.33 | 19.64 |
| 314 | 3343.39 | 31.80 |
| 315 | 1405.71 | 20.16 |
| 316 | 2587.16 | 21.07 |
| 317 | 5213.25 | 22.47 |
| 318 | 2320.16 | 20.73 |
| 319 | 4491.89 | 26.23 |
| 320 | 10199.91 | 21.11 |
| 321 | 854.38 | 34.92 |
| 322 | 1084.56 | 36.85 |
| 323 | 1814.78 | 37.29 |
| 324 | 2078.05 | 22.47 |
| 325 | 2175.08 | 33.26 |
| 326 | 2411.78 | 26.97 |
| 327 | 3738.59 | 24.76 |
| 328 | 3935.57 | 34.15 |
| 329 | 4863.21 | 26.66 |
| 330 | 860.39 | 26.25 |
| 331 | 1567.78 | 20.23 |
| 332 | 2308.11 | 27.32 |
| 333 | 2923.77 | 36.44 |
| 334 | 3295.55 | 25.40 |
| 335 | 3870.85 | 33.39 |
| 336 | 1099.56 | 21.63 |
| 337 | 1359.70 | 22.92 |
| 338 | 2059.02 | 23.12 |
| 339 | 2077.03 | 21.78 |
| 340 | 3349.34 | 35.81 |
| 341 | 8853.85 | 21.08 |
| 342 | 1734.80 | 20.24 |
| 343 | 1847.95 | 43.93 |
| 344 | 2045.95 | 34.04 |
| 345 | 2289.47 | 33.56 |
| 346 | 2421.15 | 34.74 |
| 347 | 2480.67 | 23.00 |
| 348 | 2576.25 | 34.17 |
| 349 | 3353.93 | 23.53 |
| 350 | 1083.52 | 26.24 |
| 351 | 2073.17 | 27.43 |
| 352 | 7958.65 | 34.32 |
| 353 | 1837.88 | 30.54 |
| 354 | 2939.03 | 33.75 |
| 355 | 2977.31 | 19.59 |
| 356 | 3596.46 | 21.54 |
| 657 | 3851.68 | 24.97 |
| 358 | 1135.52 | 27.83 |
| 359 | 3378.05 | 38.81 |
| 360 | 3590.72 | 28.99 |
| 361 | 3959.80 | 19.95 |
| 362 | 1258.41 | 36.10 |
| 363 | 1513.50 | 36.82 |
| 364 | 1716.38 | 20.59 |
| 365 | 2022.97 | 33.38 |
| 366 | 2914.54 | 24.29 |
| 367 | 5527.56 | 27.58 |
| 368 | 931.51 | 20.00 |
| 369 | 973.26 | 35.59 |
| 370 | 1385.67 | 27.92 |
| 371 | 2272.31 | 23.80 |
| 372 | 4024.87 | 33.20 |
| 373 | 2216.11 | 33.79 |
| 374 | 2756.23 | 35.16 |
| 375 | 2777.71 | 21.55 |
| 376 | 3521.02 | 30.73 |
| 377 | 3750.72 | 32.45 |
| 378 | 4229.09 | 29.08 |
| 379 | 4846.50 | 26.40 |
| 380 | 1046.55 | 25.35 |
| 381 | 1608.80 | 30.94 |
| 382 | 1878.78 | 31.58 |
| 383 | 2589.16 | 22.45 |
| 384 | 4369.06 | 20.25 |
| 385 | 12717.08 | 26.92 |
| 386 | 1210.43 | 36.48 |
| 387 | 3092.54 | 36.22 |
| 388 | 3248.61 | 25.65 |
| 389 | 4012.41 | 20.81 |
| 390 | 11016.34 | 21.31 |
| 391 | 1284.61 | 29.17 |
| 392 | 1460.83 | 22.53 |
| 393 | 1807.88 | 23.98 |
| 394 | 2596.33 | 34.86 |
| 395 | 2686.97 | 29.06 |
| 396 | 3871.59 | 27.51 |
| 397 | 4069.63 | 25.30 |
| 398 | 4288.98 | 25.94 |
| 399 | 4426.21 | 20.09 |
| 400 | 1071.55 | 21.41 |
| 401 | 1749.88 | 30.54 |
| 402 | 1956.97 | 21.44 |
| 403 | 2189.12 | 26.54 |
| 404 | 2257.63 | 36.10 |
| 405 | 2917.54 | 28.99 |
| 406 | 3633.69 | 26.99 |
| 407 | 6055.77 | 21.03 |
| 408 | 6186.02 | 24.99 |
| 409 | 1858.92 | 24.17 |
| 410 | 2274.11 | 33.47 |
| 411 | 4522.51 | 26.20 |
| 412 | 6237.35 | 31.39 |
| 413 | 9883.82 | 20.84 |
| 414 | 3385.6 | 25.47 |
| 415 | 3745.6 | 26.65 |
| 416 | 1408.7 | 39.13 |
| 417 | 2551.3 | 34.75 |
| 418 | 3265.3 | 36.02 |
| 419 | 2739.3 | 28.4 |
| 420 | 2065 | 24.48 |
| 421 | 2264.1 | 22.67 |
| 422 | 1058.5 | 24.94 |
| 423 | 4467.9 | 29.05 |
| 424 | 2887.4 | 35.66 |
| 425 | 1635.8 | 40.33 |
| 426 | 2525.2 | 27.72 |
| 427 | 1526.8 | 23.63 |
| 428 | 1664.8 | 29.87 |
| 429 | 2583.3 | 28.31 |
| 430 | 2663.3 | 23.44 |
| 431 | 1878.9 | 42.18 |
| 432 | 1462.7 | 39.31 |
| 433 | 1834.9 | 24 |
| 434 | 1893.1 | 24.64 |
| 435 | 1934 | 21.63 |
| 436 | 1367.7 | 38.87 |
| 437 | 1009.5 | 27.33 |
| 438 | 3405.1 | 25.92 |
| 439 | 2314.1 | 33.67 |
| 440 | 3996.8 | 20.93 |
| 441 | 2823.6 | 29.07 |
| 442 | 1179.6 | 27.15 |
| 443 | 1435.7 | 28.86 |
| 444 | 2430.7 | 35.39 |
| 445 | 1134.6 | 23.68 |
| 446 | 2014 | 25.18 |
| 447 | 2577.3 | 24.55 |
| 448 | 1194.6 | 26.73 |
| 449 | 1588.8 | 30.2 |
| 450 | 2056 | 25.44 |
| 451 | 2442.16 | 34.11 |
| 452 | 1422.66 | 21.72 |
| 453 | 1623.8 | 24.15 |
| 454 | 1624.61 | 37.73 |
| 455 | 3298.48 | 36.06 |
| 456 | 1016.31 | 35.67 |
| 457 | 1580.94 | 24.31 |
| 458 | 1157.58 | 37.41 |
| 459 | 1250.61 | 27.94 |
| 460 | 1378.67 | 28.85 |
| 461 | 1392.68 | 21.75 |
| 462 | 1409.64 | 22.06 |
| 463 | 1425.65 | 22.34 |
| 464 | 1451.71 | 29.19 |
| 465 | 1576.66 | 26.5 |
| 466 | 1651.85 | 40.6 |
| 467 | 1876.94 | 22.29 |
| 468 | 1911.12 | 24.98 |
| 469 | 2064.01 | 21.95 |
| 470 | 2150.04 | 27.76 |
| 471 | 2751.59 | 29.16 |
| 472 | 4289.94 | 28.69 |
| 473 | 4306.05 | 28.78 |
| 474 | 4800.18 | 23.83 |
| 475 | 1111.32 | 35.47 |
| 476 | 1181.49 | 36.79 |
| 477 | 3168.38 | 24.69 |
| 478 | 1229.57 | 36.29 |
| 479 | 1579.78 | 29.83 |
| 480 | 1680.82 | 30.02 |
| 481 | 1725.66 | 38.3 |
| 482 | 5228.15 | 27.04 |
| 483 | 1769.78 | 28.25 |
| 484 | 1114.54 | 25.52 |
| 485 | 1390.5 | 37.05 |
| 486 | 2046.99 | 32.56 |
| 487 | 2899.33 | 49.62 |
| 488 | 1096.53 | 26.12 |
| 489 | 1257.49 | 34.26 |
| 490 | 868.45 | 23.35 |
| 491 | 1160.43 | 35.6 |
| 492 | 1539.8 | 40.36 |
| 493 | 3318.91 | 36.01 |
| 494 | 1084.48 | 25.31 |
| 495 | 1388.39 | 58.99 |
| 496 | 3129.86 | 35.93 |
| 497 | 1255.56 | 36.33 |
| 498 | 1383.69 | 39.02 |
| 499 | 1561.75 | 40.72 |
| 500 | 3108.55 | 31.25 |
| 501 | 1173.58 | 37.51 |
| 502 | 1100.55 | 36.99 |
| 503 | 1128.44 | 33.71 |
| 504 | 3149.6 | 31.22 |
| 505 | 1068.56 | 21.69 |
| 506 | 1349.48 | 36.47 |
| 507 | 1689.81 | 40.57 |
| 508 | 2305.7 | 34.8 |
| 509 | 840.44 | 23.94 |
| 510 | 911.3 | 34.39 |
| 511 | 1299.64 | 22.42 |
| 512 | 911.47 | 25.92 |
| 513 | 1025.51 | 25.44 |
| 514 | 3400.07 | 42.03 |
| 515 | 1901.89 | 43.92 |
| 516 | 1110.42 | 34.37 |
| 517 | 1032.5 | 25.89 |
| 518 | 1040.52 | 25.11 |
| 519 | 1265.64 | 27.14 |
| 520 | 1171.55 | 29.24 |
| 521 | 1012.53 | 35.08 |
| 522 | 1286.49 | 36.78 |
| 523 | 2932.36 | 34.11 |
| 524 | 1215.49 | 27.61 |
| 525 | 1423.68 | 21.47 |
| 526 | 1487.71 | 29.58, |

wherein said CE times are based on capillary electrophoresis using a glass capillary of 90 cm in length with an inner diameter (ID) of 50 μm at an applied voltage of 30 kV,
wherein 20% acetonitrile and 0.25% formic acid in water is used as the mobile phase for the capillary electrophoresis at a flow rate of 2 μl/min., and
wherein said CE times are calibrated relative to the following values:

| Protein/Polypeptide | Migration time (minutes) |
|---|---|
| Aprotinin | 9.2 |
| Ribonuclease | 10.9 |
| Lysozyme | 8.9 |
| “REV” SEQ ID NO: 1 | 15.6 |
| “ELM” SEQ ID NO: 2 | 23.4 |
| “KINCON” SEQ ID NO: 3 | 20.0 |
| “GIVLY” SEQ ID NO: 4 | 36.8; |

determining if said subset of polypeptide markers comprises at least a first, second and third biomarker selected from:
a first biomarker that is at least one selected from the group consisting of: (1) markers 1 to 106 (frequency markers) having (a) a frequency of presence in patients with VD of at least 0.4 and (b) a frequency of presence in patients with VD at least three (3) times the frequency of presence in patients without VD; (2) markers 1 to 106 (frequency markers) having (a) a frequency of presence in patients without VD of at least 0.4 and (b) a frequency of presence in patients without VD at least three (3) times the frequency of presence in patients with VD; (3) markers 107 to 526 (amplitude markers) having a mean amplitude in patients with VD of at least 200 and a mean amplitude at least two (2) times greater than the mean amplitude of the same marker in patients without VD; and (4) markers 107 to 526 (amplitude markers) having a mean amplitude in patients without VD of at least 200 and a mean amplitude at least two (2) times greater than the mean amplitude of the same marker in patients with VD;
a second biomarker which is different than said first biomarker and is at least one selected from the group consisting of: (1) markers 1 to 106 (frequency markers) having (a) a frequency of presence in patients with VD of at least 0.4 and (b) a frequency of presence in patients with VD at least three (3) times the frequency of presence in patients without VD; (2) markers 1 to 106 (frequency markers) having (a) a frequency of presence in patients without VD of at least 0.4 and (b) a frequency of presence in patients without VD at least three (3) times the frequency of presence in patients with VD; (3) markers 107 to 526 (amplitude markers) having a mean amplitude in patients with VD of at least 200 and a mean amplitude at least two (2) times greater than the mean amplitude of the same marker in patients without VD; and (4) markers 107 to 526 (amplitude markers) having a mean amplitude in patients without VD of at least 200 and a mean amplitude at least two (2) times greater than the mean amplitude of the same marker in patients with VD; and
a third biomarker which is different than said first and second biomarkers and is at least one selected from the group consisting of markers 1 to 526 (frequency and amplitude markers);
comparing the frequency and/or amplitude of presence of said at least first, second and third biomarkers in said sample of said subject patient to the frequency and/or amplitude of presence of the same at least first, second and third biomarkers from said known polypeptide markers from said control subjects,
ranking said subject patient between said control subjects with VD and without VD based on the second comparing step; and
diagnosing the probability of VD in said subject patient based on said ranking.

12. The method according to claim 1, wherein the frequency of said frequency markers 1 to 106 is compared with the following reference values:

| | Occurrence | Occurrence |
|---|---|---|
| No. | control | CVD |
| 1 | 0.01 | 0.58 |
| 2 | 0.18 | 0.75 |
| 3 | 0.14 | 0.67 |
| 4 | 0.25 | 0.75 |
| 5 | 0.08 | 0.58 |
| 6 | 0.05 | 0.54 |
| 7 | 0.36 | 0.83 |
| 8 | 0.31 | 0.79 |
| 9 | 0.26 | 0.71 |
| 10 | 0.14 | 0.58 |
| 11 | 0.06 | 0.50 |
| 12 | 0.15 | 0.58 |
| 13 | 0.31 | 0.75 |
| 14 | 0.32 | 0.75 |
| 15 | 0.12 | 0.54 |
| 16 | 0.03 | 0.46 |
| 17 | 0.25 | 0.67 |
| 18 | 0.18 | 0.58 |
| 19 | 0.47 | 0.88 |
| 20 | 0.18 | 0.58 |
| 21 | 0.09 | 0.50 |
| 22 | 0.27 | 0.67 |
| 23 | 0.18 | 0.58 |
| 24 | 0.10 | 0.50 |
| 25 | 0.44 | 0.04 |
| 26 | 0.44 | 0.04 |
| 27 | 0.44 | 0.04 |
| 28 | 0.48 | 0.08 |
| 29 | 0.74 | 0.33 |
| 30 | 0.62 | 0.21 |
| 31 | 0.45 | 0.04 |
| 32 | 0.41 | 0.00 |
| 33 | 0.62 | 0.21 |
| 34 | 0.74 | 0.33 |
| 35 | 0.41 | 0.00 |
| 36 | 0.78 | 0.38 |
| 37 | 0.45 | 0.04 |
| 38 | 0.70 | 0.29 |
| 39 | 0.78 | 0.38 |
| 40 | 0.41 | 0.00 |
| 41 | 0.67 | 0.25 |
| 42 | 0.59 | 0.17 |
| 43 | 0.67 | 0.25 |
| 44 | 0.54 | 0.13 |
| 45 | 0.63 | 0.21 |
| 46 | 0.42 | 0.00 |
| 47 | 0.80 | 0.38 |
| 48 | 0.54 | 0.13 |
| 49 | 0.42 | 0.00 |
| 50 | 0.58 | 0.17 |
| 51 | 0.42 | 0.00 |
| 52 | 0.72 | 0.29 |
| 53 | 0.52 | 0.08 |
| 54 | 0.52 | 0.08 |
| 55 | 0.59 | 0.17 |
| 56 | 0.65 | 0.21 |
| 57 | 0.48 | 0.04 |
| 58 | 0.57 | 0.13 |
| 59 | 0.56 | 0.13 |
| 60 | 0.44 | 0.00 |
| 61 | 0.57 | 0.13 |
| 62 | 0.44 | 0.00 |
| 63 | 0.73 | 0.29 |
| 64 | 0.53 | 0.08 |
| 65 | 0.50 | 0.04 |
| 66 | 0.49 | 0.04 |
| 67 | 0.70 | 0.25 |
| 68 | 0.91 | 0.46 |
| 69 | 0.71 | 0.25 |
| 70 | 0.63 | 0.17 |
| 71 | 0.47 | 0.00 |
| 72 | 0.81 | 0.33 |
| 73 | 0.59 | 0.13 |
| 74 | 0.90 | 0.42 |
| 75 | 0.77 | 0.29 |
| 76 | 0.94 | 0.46 |
| 77 | 0.69 | 0.21 |
| 78 | 0.49 | 0.00 |
| 79 | 0.66 | 0.17 |
| 80 | 0.53 | 0.04 |
| 81 | 0.53 | 0.04 |
| 82 | 0.67 | 0.17 |
| 83 | 0.50 | 0.00 |
| 84 | 0.50 | 0.00 |
| 85 | 0.62 | 0.13 |
| 86 | 0.88 | 0.38 |
| 87 | 0.63 | 0.13 |
| 88 | 0.55 | 0.04 |
| 89 | 0.52 | 0.00 |
| 90 | 0.61 | 0.08 |
| 91 | 0.91 | 0.38 |
| 92 | 0.75 | 0.21 |
| 93 | 0.67 | 0.13 |
| 94 | 0.73 | 0.17 |
| 95 | 0.60 | 0.04 |
| 96 | 0.69 | 0.13 |
| 97 | 0.85 | 0.29 |
| 98 | 0.60 | 0.04 |
| 99 | 0.70 | 0.13 |
| 100 | 0.71 | 0.13 |
| 101 | 0.58 | 0.00 |
| 102 | 0.59 | 0.00 |
| 103 | 0.80 | 0.17 |
| 104 | 0.77 | 0.08 |
| 105 | 0.98 | 0.49 |
| 106 | 0.90 | 0.49 |

wherein the amplitude of said amplitude markers 107 to 413 is compared with the following reference values:

| | Mean | |
|---|---|---|
| | amplitude | Mean |
| | control | amplitude |
| No. | group | CVD group |
| 107 | 94 | 253 |
| 108 | 116 | 233 |
| 109 | 50 | 123 |
| 110 | 766 | 1878 |
| 111 | 45 | 175 |
| 112 | 89 | 419 |
| 113 | 69 | 146 |
| 114 | 174 | 418 |
| 115 | 78 | 689 |
| 116 | 47 | 99 |
| 117 | 59 | 188 |
| 118 | 120 | 357 |
| 119 | 317 | 2460 |
| 120 | 121 | 463 |
| 121 | 172 | 380 |
| 122 | 796 | 1674 |
| 123 | 167 | 888 |
| 124 | 1703 | 636 |
| 125 | 768 | 3651 |
| 126 | 340 | 1283 |
| 127 | 193 | 583 |
| 128 | 135 | 320 |
| 129 | 243 | 566 |
| 130 | 95 | 214 |
| 131 | 161 | 768 |
| 132 | 118 | 299 |
| 133 | 116 | 267 |
| 134 | 840 | 1950 |
| 135 | 102 | 288 |
| 136 | 127 | 283 |
| 137 | 5726 | 12044 |
| 138 | 263 | 728 |
| 139 | 506 | 154 |
| 140 | 113 | 289 |
| 141 | 150 | 301 |
| 142 | 136 | 290 |
| 143 | 51 | 189 |
| 144 | 168 | 343 |
| 145 | 196 | 769 |
| 146 | 119 | 250 |
| 147 | 161 | 358 |
| 148 | 227 | 58 |
| 149 | 130 | 289 |
| 150 | 97 | 196 |
| 151 | 192 | 504 |
| 152 | 301 | 128 |
| 153 | 442 | 108 |
| 154 | 154 | 1119 |
| 155 | 197 | 725 |
| 156 | 82 | 201 |
| 157 | 100 | 202 |
| 158 | 189 | 513 |
| 159 | 1054 | 486 |
| 160 | 161 | 412 |
| 161 | 123 | 456 |
| 162 | 229 | 517 |
| 163 | 273 | 554 |
| 164 | 196 | 517 |
| 165 | 197 | 97 |
| 166 | 176 | 506 |
| 167 | 1480 | 686 |
| 168 | 3107 | 880 |
| 169 | 80 | 216 |
| 170 | 203 | 1328 |
| 171 | 344 | 848 |
| 172 | 797 | 206 |
| 173 | 1146 | 2842 |
| 174 | 224 | 568 |
| 175 | 138 | 450 |
| 176 | 258 | 525 |
| 177 | 15571 | 7296 |
| 178 | 367 | 745 |
| 179 | 185 | 575 |
| 180 | 260 | 130 |
| 181 | 1492 | 3433 |
| 182 | 784 | 351 |
| 183 | 1158 | 2826 |
| 184 | 919 | 371 |
| 185 | 156 | 466 |
| 186 | 1694 | 4348 |
| 187 | 201 | 66 |
| 188 | 1787 | 737 |
| 189 | 2810 | 6060 |
| 190 | 1703 | 766 |
| 191 | 461 | 1095 |
| 192 | 707 | 6865 |
| 193 | 493 | 1490 |
| 194 | 346 | 799 |
| 195 | 3338 | 9120 |
| 196 | 240 | 654 |
| 197 | 80 | 203 |
| 198 | 490 | 236 |
| 199 | 259 | 533 |
| 200 | 1142 | 421 |
| 201 | 1241 | 2506 |
| 202 | 1511 | 749 |
| 203 | 294 | 107 |
| 204 | 1090 | 2230 |
| 205 | 1151 | 456 |
| 206 | 983 | 475 |
| 207 | 220 | 44 |
| 208 | 1195 | 546 |
| 209 | 3909 | 1825 |
| 210 | 406 | 167 |
| 211 | 149 | 58 |
| 212 | 1098 | 2400 |
| 213 | 769 | 164 |
| 214 | 527 | 1675 |
| 215 | 1173 | 416 |
| 216 | 711 | 324 |
| 217 | 470 | 181 |
| 218 | 723 | 333 |
| 219 | 213 | 102 |
| 220 | 345 | 169 |
| 221 | 677 | 334 |
| 222 | 2489 | 744 |
| 223 | 132 | 63 |
| 224 | 1451 | 717 |
| 225 | 1324 | 299 |
| 226 | 1689 | 741 |
| 227 | 88 | 238 |
| 228 | 191 | 701 |
| 229 | 361 | 118 |
| 230 | 5095 | 1789 |
| 231 | 601 | 241 |
| 232 | 1200 | 403 |
| 233 | 736 | 245 |
| 234 | 1171 | 297 |
| 235 | 678 | 263 |
| 236 | 1597 | 482 |
| 237 | 115 | 353 |
| 238 | 392 | 146 |
| 239 | 120 | 265 |
| 240 | 1127 | 465 |
| 241 | 623 | 140 |
| 242 | 250 | 115 |
| 243 | 633 | 306 |
| 244 | 224 | 81 |
| 245 | 120 | 60 |
| 246 | 1275 | 438 |
| 247 | 283 | 89 |
| 248 | 1514 | 737 |
| 249 | 264 | 91 |
| 250 | 900 | 278 |
| 251 | 776 | 338 |
| 252 | 411 | 97 |
| 253 | 227 | 103 |
| 254 | 186 | 53 |
| 255 | 890 | 332 |
| 256 | 469 | 1170 |
| 257 | 152 | 70 |
| 258 | 21200 | 7156 |
| 259 | 282 | 115 |
| 260 | 474 | 152 |
| 261 | 117 | 274 |
| 262 | 1359 | 517 |
| 263 | 421 | 195 |
| 264 | 183 | 83 |
| 265 | 151 | 64 |
| 266 | 206 | 99 |
| 267 | 588 | 256 |
| 268 | 304 | 119 |
| 269 | 147 | 61 |
| 270 | 172 | 66 |
| 271 | 338 | 157 |
| 272 | 292 | 138 |
| 273 | 227 | 110 |
| 274 | 142 | 387 |
| 275 | 166 | 79 |
| 276 | 179 | 385 |
| 277 | 200 | 75 |
| 278 | 211 | 81 |
| 279 | 169 | 68 |
| 280 | 359 | 157 |
| 281 | 141 | 284 |
| 282 | 244 | 104 |
| 283 | 882 | 331 |
| 284 | 903 | 324 |
| 285 | 231 | 98 |
| 286 | 1420 | 457 |
| 287 | 2096 | 591 |
| 288 | 676 | 261 |
| 289 | 470 | 234 |
| 290 | 169 | 49 |
| 291 | 234 | 517 |
| 292 | 624 | 309 |
| 293 | 279 | 111 |
| 294 | 444 | 130 |
| 295 | 752 | 1640 |
| 296 | 543 | 191 |
| 297 | 164 | 66 |
| 298 | 785 | 274 |
| 299 | 185 | 79 |
| 300 | 234 | 99 |
| 301 | 179 | 46 |
| 302 | 360 | 141 |
| 303 | 106 | 37 |
| 304 | 146 | 47 |
| 305 | 730 | 323 |
| 306 | 373 | 40 |
| 307 | 97 | 39 |
| 308 | 48 | 104 |
| 309 | 176 | 375 |
| 310 | 87 | 185 |
| 311 | 187 | 91 |
| 312 | 143 | 43 |
| 313 | 381 | 143 |
| 314 | 402 | 194 |
| 315 | 237 | 113 |
| 316 | 519 | 207 |
| 317 | 115 | 56 |
| 318 | 197 | 61 |
| 319 | 254 | 1335 |
| 320 | 283 | 140 |
| 321 | 88 | 201 |
| 322 | 119 | 56 |
| 323 | 129 | 46 |
| 324 | 125 | 50 |
| 325 | 3240 | 7677 |
| 326 | 114 | 51 |
| 327 | 236 | 89 |
| 328 | 163 | 79 |
| 329 | 702 | 204 |
| 330 | 481 | 159 |
| 331 | 407 | 175 |
| 332 | 228 | 79 |
| 333 | 200 | 98 |
| 334 | 356 | 80 |
| 335 | 152 | 72 |
| 336 | 178 | 64 |
| 337 | 281 | 50 |
| 338 | 293 | 104 |
| 339 | 796 | 299 |
| 340 | 174 | 83 |
| 341 | 1025 | 194 |
| 342 | 209 | 95 |
| 343 | 407 | 145 |
| 344 | 144 | 462 |
| 345 | 182 | 74 |
| 346 | 95 | 42 |
| 347 | 92 | 16 |
| 348 | 150 | 36 |
| 349 | 256 | 96 |
| 350 | 130 | 51 |
| 351 | 96 | 46 |
| 352 | 330 | 157 |
| 353 | 248 | 107 |
| 354 | 205 | 69 |
| 355 | 310 | 36 |
| 356 | 411 | 139 |
| 357 | 789 | 179 |
| 358 | 263 | 104 |
| 359 | 184 | 84 |
| 360 | 206 | 52 |
| 361 | 755 | 287 |
| 362 | 246 | 26 |
| 363 | 316 | 106 |
| 364 | 1329 | 142 |
| 365 | 122 | 23 |
| 366 | 105 | 46 |
| 367 | 311 | 127 |
| 368 | 131 | 56 |
| 369 | 206 | 38 |
| 370 | 104 | 41 |
| 371 | 126 | 43 |
| 372 | 345 | 110 |
| 373 | 416 | 151 |
| 374 | 209 | 86 |
| 375 | 268 | 54 |
| 376 | 549 | 188 |
| 377 | 115 | 36 |
| 378 | 353 | 110 |
| 379 | 379 | 135 |
| 380 | 503 | 52 |
| 381 | 753 | 360 |
| 382 | 335 | 2617 |
| 383 | 97 | 31 |
| 384 | 1280 | 178 |
| 385 | 438 | 64 |
| 386 | 374 | 92 |
| 387 | 329 | 109 |
| 388 | 283 | 124 |
| 389 | 273 | 36 |
| 390 | 3045 | 864 |
| 391 | 51 | 25 |
| 392 | 711 | 69 |
| 393 | 187 | 82 |
| 394 | 74 | 29 |
| 395 | 197 | 61 |
| 396 | 320 | 100 |
| 397 | 712 | 156 |
| 398 | 187 | 48 |
| 399 | 337 | 7 |
| 400 | 133 | 59 |
| 401 | 297 | 110 |
| 402 | 164 | 55 |
| 403 | 876 | 4574 |
| 404 | 820 | 309 |
| 405 | 845 | 288 |
| 406 | 475 | 119 |
| 407 | 275 | 91 |
| 408 | 1590 | 291 |
| 409 | 1343 | 334 |
| 410 | 180 | 61 |
| 411 | 149 | 13 |
| 412 | 298 | 135 |
| 413 | 469 | 42 |

and wherein the amplitude of said amplitude markers 414 to 526 is compared with the following reference values:

| | Mean amplitude | Mean amplitude |
|---|---|---|
| No. | control group | CVD group |
| 414 | 3214 | 2678 |
| 415 | 514 | 250 |
| 416 | 1359 | 615 |
| 417 | 581 | 174 |
| 418 | 630 | 499 |
| 419 | 212 | 141 |
| 420 | 681 | 381 |
| 421 | 445 | 227 |
| 422 | 1178 | 103 |
| 423 | 540 | 348 |
| 424 | 188 | 206 |
| 425 | 1540 | 687 |
| 426 | 569 | 914 |
| 427 | 301 | 106 |
| 428 | 976 | 511 |
| 429 | 972 | 515 |
| 430 | 1320 | 729 |
| 431 | 278 | 210 |
| 432 | 1682 | 1196 |
| 433 | 589 | 287 |
| 434 | 384 | 502 |
| 435 | 1006 | 399 |
| 436 | 1064 | 800 |
| 437 | 270 | 216 |
| 438 | 3453 | 2235 |
| 439 | 837 | 790 |
| 440 | 1353 | 684 |
| 441 | 710 | 733 |
| 442 | 809 | 627 |
| 443 | 8328 | 3904 |
| 444 | 596 | 661 |
| 445 | 380 | 593 |
| 446 | 2389 | 1375 |
| 447 | 297 | 285 |
| 448 | 4154 | 2314 |
| 449 | 532 | 953 |
| 450 | 1145 | 733 |
| 451 | 744 | 845 |
| 452 | 2878 | 2433 |
| 453 | 8725 | 4307 |
| 454 | 1109 | 1620 |
| 455 | 750 | 409 |
| 456 | 687 | 971 |
| 457 | 2155 | 1229 |
| 458 | 1622 | 1964 |
| 459 | 50512 | 45582 |
| 460 | 2259 | 2915 |
| 461 | 4142 | 4664 |
| 462 | 6265 | 8932 |
| 463 | 1969 | 2931 |
| 464 | 36818 | 19376 |
| 465 | 1352 | 1591 |
| 466 | 5789 | 1562 |
| 467 | 2562 | 1358 |
| 468 | 91735 | 90455 |
| 469 | 7723 | 4053 |
| 470 | 2496 | 1342 |
| 471 | 358 | 423 |
| 472 | 3360 | 3317 |
| 473 | 4000 | 2575 |
| 474 | 1388 | 2785 |
| 475 | 335 | 3122 |
| 476 | 302 | 237 |
| 477 | 351 | 847 |
| 478 | 186 | 145 |
| 479 | 3094 | 2397 |
| 480 | 4737 | 2446 |
| 481 | 1468 | 2644 |
| 482 | 566 | 974 |
| 483 | 535 | 429 |
| 484 | 2818 | 4530 |
| 485 | 17423 | 37226 |
| 486 | 3087 | 1793 |
| 487 | 25 | 319 |
| 488 | 3397 | 6633 |
| 489 | 2904 | 6138 |
| 490 | 239 | 198 |
| 491 | 1794 | 3083 |
| 492 | 2558 | 1701 |
| 493 | 428 | 419 |
| 494 | 1326 | 2891 |
| 495 | 181 | 788 |
| 496 | 212 | 207 |
| 497 | 741 | 600 |
| 498 | 135 | 197 |
| 499 | 4632 | 4647 |
| 500 | 331 | 461 |
| 501 | 302 | 414 |
| 502 | 206 | 306 |
| 503 | 1521 | 3346 |
| 504 | 349 | 561 |
| 505 | 211 | 315 |
| 506 | 208 | 247 |
| 507 | 1270 | 1039 |
| 508 | 305 | 334 |
| 509 | 213 | 266 |
| 510 | 2436 | 3827 |
| 511 | 460 | 294 |
| 512 | 389 | 924 |
| 513 | 197 | 273 |
| 514 | 152 | 448 |
| 515 | 743 | 575 |
| 516 | 428 | 713 |
| 517 | 186 | 298 |
| 518 | 219 | 296 |
| 519 | 4218 | 7618 |
| 520 | 75 | 148 |
| 521 | 96 | 214 |
| 522 | 56 | 92 |
| 523 | 208 | 316 |
| 524 | 349 | 729 |
| 525 | 543 | 1220 |
| 526 | 388 | 684 |

13. A method of diagnosing vascular diseases (VD) in a subject patient, comprising:
obtaining a urine sample from said subject;
pretreating said urine sample by a method selected from the group consisting of purification, separation, dilution, concentration or any combinations of the foregoing;
purifying said pretreated urine sample to remove high concentration proteins;
separating said pretreated and purified urine sample into a plurality of polypeptides using a combination of mass spectrometry (MS) and capillary electrophoresis (CE);
identifying said plurality of pretreated and purified polypeptides based on the separation characteristics of each of said plurality of pretreated and purified polypeptides in the separating step, wherein said separation characteristics comprise molecular mass and elution time;
comparing said plurality of pretreated and purified polypeptides to known polypeptide markers taken from control subjects with VD and without VD to obtain a subset of polypeptide markers from said plurality of pretreated and purified polypeptides that substantially match said known polypeptide markers, wherein said known polypeptide markers are characterized by the following molecular masses and migration times (CE time):

| No. | Mass | CE time |
|---|---|---|
| 1 | 1166.61 | 23.88 |
| 2 | 2431.50 | 24.10 |
| 3 | 1922.93 | 31.99 |
| 4 | 2509.16 | 25.76 |
| 5 | 3194.22 | 30.34 |
| 6 | 1705.80 | 40.47 |
| 7 | 1962.95 | 31.77 |
| 8 | 3822.12 | 24.72 |
| 9 | 2212.32 | 24.94 |
| 10 | 3015.78 | 35.86 |
| 11 | 1784.95 | 20.94 |
| 12 | 1902.92 | 31.87 |
| 13 | 2329.15 | 27.17 |
| 14 | 2154.05 | 21.78 |
| 15 | 2166.03 | 27.89 |
| 16 | 2258.27 | 21.99 |
| 17 | 2573.84 | 20.49 |
| 18 | 1270.75 | 37.92 |
| 19 | 1611.84 | 40.12 |
| 20 | 1791.87 | 41.04 |
| 21 | 2030.00 | 25.23 |
| 22 | 1290.40 | 30.87 |
| 23 | 1441.74 | 39.13 |
| 24 | 2924.25 | 24.05 |
| 25 | 816.41 | 21.10 |
| 26 | 963.52 | 21.71 |
| 27 | 1503.74 | 29.63 |
| 28 | 2849.59 | 23.02 |
| 29 | 3133.20 | 31.20 |
| 30 | 1283.62 | 27.30 |
| 31 | 1495.75 | 23.31 |
| 32 | 1513.70 | 29.29 |
| 33 | 1612.83 | 23.36 |
| 34 | 2319.19 | 33.80 |
| 35 | 2436.23 | 22.87 |
| 36 | 2557.42 | 28.22 |
| 37 | 2626.85 | 28.00 |
| 38 | 2933.46 | 27.68 |
| 39 | 2994.09 | 29.50 |
| 40 | 4101.34 | 28.51 |
| 41 | 935.49 | 23.69 |
| 42 | 1521.75 | 30.42 |
| 43 | 1669.79 | 21.48 |
| 44 | 2758.37 | 28.94 |
| 45 | 3546.94 | 26.22 |
| 46 | 3609.63 | 20.22 |
| 47 | 3697.49 | 23.71 |
| 48 | 4278.73 | 23.34 |
| 49 | 4421.04 | 20.73 |
| 50 | 4805.67 | 26.49 |
| 51 | 11058.18 | 21.88 |
| 52 | 1352.61 | 29.86 |
| 53 | 2802.85 | 36.35 |
| 54 | 4890.88 | 26.48 |
| 55 | 5212.06 | 26.98 |
| 56 | 945.45 | 25.80 |
| 57 | 1065.55 | 25.50 |
| 58 | 1137.58 | 26.41 |
| 59 | 1542.77 | 23.91 |
| 60 | 1693.83 | 23.47 |
| 61 | 3361.42 | 24.26 |
| 62 | 3617.74 | 26.97 |
| 63 | 3737.69 | 37.15 |
| 64 | 980.54 | 22.44 |
| 65 | 1221.63 | 26.82 |
| 66 | 2952.27 | 25.14 |
| 67 | 3696.88 | 26.94 |
| 68 | 5574.45 | 23.24 |
| 69 | 1182.59 | 28.34 |
| 70 | 1963.96 | 31.76 |
| 71 | 882.54 | 23.81 |
| 72 | 4002.72 | 20.69 |
| 73 | 4059.96 | 20.44 |
| 74 | 1186.59 | 22.31 |
| 75 | 1825.87 | 31.80 |
| 76 | 3401.66 | 23.42 |
| 77 | 1496.75 | 30.36 |
| 78 | 1832.92 | 31.91 |
| 79 | 2281.35 | 36.34 |
| 80 | 2344.34 | 33.66 |
| 81 | 3944.82 | 24.59 |
| 82 | 3002.23 | 23.80 |
| 83 | 3416.77 | 36.76 |
| 84 | 3501.86 | 31.79 |
| 85 | 6783.03 | 26.61 |
| 86 | 14111.27 | 21.97 |
| 87 | 2616.02 | 28.35 |
| 88 | 2810.45 | 36.73 |
| 89 | 2940.95 | 29.07 |
| 30 | 2946.45 | 34.96 |
| 91 | 1494.72 | 30.40 |
| 92 | 1080.53 | 27.86 |
| 33 | 2349.14 | 27.36 |
| 94 | 3303.00 | 23.07 |
| 95 | 4081.56 | 24.51 |
| 96 | 4670.27 | 25.84 |
| 97 | 4671.99 | 23.33 |
| 98 | 8933.94 | 22.57 |
| 99 | 1523.90 | 29.72 |
| 100 | 3956.82 | 25.20 |
| 101 | 3984.81 | 21.29 |
| 102 | 4830.78 | 26.61 |
| 103 | 3031.39 | 35.93 |
| 104 | 3788.76 | 25.21 |
| 105 | 2567.2 | 34.76 |
| 106 | 1447.8 | 19.49 |
| 107 | 2241.51 | 24.11 |
| 108 | 2461.11 | 30.84 |
| 109 | 1965.96 | 23.62 |
| 110 | 2189.08 | 27.17 |
| 111 | 1127.58 | 20.82 |
| 112 | 1400.71 | 20.35 |
| 113 | 1512.75 | 39.51 |
| 114 | 1860.53 | 34.24 |
| 115 | 1442.69 | 27.72 |
| 116 | 2590.78 | 27.96 |
| 117 | 1556.72 | 27.90 |
| 118 | 2309.15 | 21.95 |
| 119 | 2389.33 | 22.34 |
| 120 | 1478.68 | 39.28 |
| 121 | 1795.90 | 24.66 |
| 122 | 2211.03 | 35.06 |
| 123 | 1223.63 | 19.52 |
| 124 | 1829.04 | 21.22 |
| 125 | 1878.66 | 30.19 |
| 126 | 2009.96 | 32.27 |
| 127 | 2110.00 | 24.10 |
| 128 | 1552.79 | 29.75 |
| 129 | 1577.75 | 40.03 |
| 130 | 1936.94 | 34.71 |
| 131 | 2368.13 | 26.75 |
| 132 | 3633.05 | 33.25 |
| 133 | 1510.72 | 28.30 |
| 134 | 1668.87 | 40.49 |
| 135 | 2227.05 | 33.43 |
| 136 | 1495.75 | 39.41 |
| 137 | 1631.77 | 45.38 |
| 138 | 3158.60 | 29.62 |
| 139 | 1522.78 | 22.76 |
| 140 | 1727.87 | 39.61 |
| 141 | 1883.94 | 40.14 |
| 142 | 1460.71 | 19.83 |
| 143 | 1805.88 | 29.92 |
| 144 | 1898.93 | 40.30 |
| 145 | 2237.06 | 27.12 |
| 146 | 3178.33 | 30.26 |
| 147 | 1844.56 | 34.28 |
| 148 | 1378.57 | 37.16 |
| 149 | 1764.86 | 29.88 |
| 150 | 1791.88 | 30.77 |
| 151 | 2082.01 | 33.67 |
| 152 | 1768.90 | 20.77 |
| 153 | 3442.09 | 33.32 |
| 154 | 876.42 | 35.07 |
| 155 | 2352.14 | 26.74 |
| 156 | 937.50 | 34.12 |
| 157 | 1445.72 | 28.36 |
| 158 | 1893.10 | 28.85 |
| 159 | 2839.43 | 24.14 |
| 160 | 1600.76 | 29.61 |
| 161 | 1565.75 | 26.35 |
| 162 | 1627.76 | 29.47 |
| 163 | 1812.90 | 39.98 |
| 164 | 3137.52 | 30.29 |
| 165 | 1364.67 | 28.65 |
| 166 | 2298.07 | 33.82 |
| 167 | 3017.74 | 49.66 |
| 168 | 1235.61 | 26.67 |
| 169 | 1741.81 | 30.21 |
| 170 | 1818.90 | 30.93 |
| 171 | 1892.95 | 22.22 |
| 172 | 3280.69 | 22.69 |
| 173 | 2658.34 | 19.50 |
| 174 | 1407.71 | 27.46 |
| 175 | 1622.79 | 26.82 |
| 176 | 1684.78 | 29.64 |
| 177 | 1321.65 | 28.39 |
| 178 | 1350.68 | 27.13 |
| 179 | 1549.76 | 39.52 |
| 180 | 2233.10 | 22.47 |
| 181 | 2679.27 | 23.48 |
| 182 | 1835.79 | 20.02 |
| 183 | 3421.66 | 25.96 |
| 184 | 1708.85 | 30.44 |
| 185 | 1993.96 | 32.16 |
| 186 | 2695.31 | 23.46 |
| 187 | 1204.65 | 21.93 |
| 188 | 1467.86 | 24.38 |
| 189 | 1767.07 | 24.10 |
| 190 | 8176.30 | 19.57 |
| 191 | 1143.56 | 36.97 |
| 192 | 1834.90 | 31.05 |
| 193 | 2025.95 | 32.21 |
| 194 | 3489.70 | 31.45 |
| 195 | 1268.62 | 27.29 |
| 196 | 1659.82 | 29.35 |
| 197 | 2405.59 | 22.16 |
| 198 | 2483.21 | 27.54 |
| 199 | 2599.21 | 28.20 |
| 200 | 4170.01 | 33.51 |
| 201 | 1013.41 | 25.33 |
| 202 | 1016.49 | 25.88 |
| 203 | 1493.74 | 22.06 |
| 204 | 2104.04 | 32.97 |
| 205 | 3718.81 | 32.39 |
| 206 | 4251.98 | 28.66 |
| 207 | 4538.67 | 26.20 |
| 208 | 2067.93 | 20.68 |
| 209 | 2292.11 | 27.26 |
| 210 | 3702.39 | 32.39 |
| 211 | 965.46 | 27.84 |
| 212 | 2186.07 | 25.89 |
| 213 | 2584.29 | 35.08 |
| 214 | 2841.13 | 24.50 |
| 215 | 9866.78 | 20.85 |
| 216 | 1099.53 | 28.33 |
| 217 | 2471.25 | 34.69 |
| 218 | 3734.85 | 32.41 |
| 219 | 3927.86 | 33.50 |
| 220 | 3166.32 | 22.10 |
| 221 | 2339.08 | 33.95 |
| 222 | 2563.76 | 22.05 |
| 223 | 3219.35 | 35.00 |
| 224 | 3359.66 | 31.84 |
| 225 | 4097.98 | 24.59 |
| 226 | 4654.14 | 25.81 |
| 227 | 1584.77 | 29.72 |
| 228 | 2148.10 | 25.54 |
| 229 | 2639.45 | 21.33 |
| 230 | 3013.27 | 22.27 |
| 231 | 3205.39 | 19.71 |
| 232 | 3831.86 | 28.39 |
| 233 | 1050.52 | 27.03 |
| 234 | 2157.06 | 22.19 |
| 235 | 2407.16 | 27.65 |
| 236 | 2837.93 | 23.99 |
| 237 | 3058.02 | 30.20 |
| 238 | 1658.67 | 21.53 |
| 239 | 3311.32 | 24.46 |
| 240 | 3556.63 | 23.64 |
| 241 | 1085.50 | 21.70 |
| 242 | 1199.63 | 21.91 |
| 243 | 1247.58 | 22.00 |
| 244 | 1608.76 | 22.36 |
| 245 | 2501.20 | 34.30 |
| 246 | 3021.52 | 23.52 |
| 247 | 4153.75 | 33.41 |
| 248 | 5000.17 | 24.43 |
| 249 | 8917.48 | 22.53 |
| 250 | 1750.86 | 23.80 |
| 251 | 2235.13 | 34.10 |
| 252 | 2644.25 | 21.13 |
| 253 | 3943.96 | 33.53 |
| 254 | 11967.96 | 20.50 |
| 255 | 2553.23 | 34.14 |
| 256 | 1209.58 | 26.31 |
| 257 | 1899.94 | 21.41 |
| 258 | 1680.00 | 23.77 |
| 259 | 2195.06 | 20.15 |
| 260 | 3064.41 | 20.55 |
| 261 | 3554.07 | 31.11 |
| 262 | 3686.03 | 22.16 |
| 263 | 3802.12 | 33.10 |
| 264 | 4048.05 | 25.42 |
| 265 | 2380.16 | 36.48 |
| 266 | 1352.83 | 24.38 |
| 267 | 1638.80 | 20.26 |
| 268 | 2864.18 | 20.19 |
| 269 | 3754.66 | 37.16 |
| 270 | 4185.91 | 33.47 |
| 271 | 858.42 | 23.26 |
| 272 | 1159.64 | 26.05 |
| 273 | 1407.71 | 37.25 |
| 274 | 1439.72 | 29.62 |
| 275 | 1720.76 | 19.72 |
| 276 | 1846.93 | 32.04 |
| 277 | 3177.14 | 22.48 |
| 278 | 4113.80 | 24.58 |
| 279 | 2744.07 | 35.03 |
| 280 | 2767.26 | 21.52 |
| 281 | 1310.64 | 27.11 |
| 282 | 1613.88 | 23.95 |
| 283 | 1703.90 | 33.64 |
| 284 | 2761.40 | 21.46 |
| 285 | 3242.42 | 22.78 |
| 286 | 3338.17 | 23.36 |
| 287 | 3371.74 | 22.96 |
| 288 | 3593.53 | 20.25 |
| 289 | 3677.52 | 24.49 |
| 290 | 1624.80 | 30.81 |
| 291 | 2210.92 | 37.55 |
| 292 | 3290.37 | 24.12 |
| 293 | 4413.76 | 29.03 |
| 294 | 1482.73 | 22.47 |
| 295 | 1813.78 | 31.87 |
| 296 | 1934.87 | 20.04 |
| 297 | 2249.89 | 34.14 |
| 298 | 3280.59 | 25.76 |
| 299 | 1098.56 | 21.46 |
| 300 | 1125.58 | 21.76 |
| 301 | 1649.79 | 19.59 |
| 302 | 4025.68 | 20.73 |
| 303 | 980.33 | 35.59 |
| 304 | 1096.41 | 35.95 |
| 305 | 1698.65 | 37.60 |
| 306 | 2361.21 | 20.77 |
| 307 | 3148.50 | 24.22 |
| 308 | 3157.23 | 34.74 |
| 309 | 1304.59 | 27.95 |
| 310 | 3575.78 | 32.27 |
| 311 | 1510.75 | 20.12 |
| 312 | 2485.20 | 34.25 |
| 313 | 3076.33 | 19.64 |
| 314 | 3343.39 | 31.80 |
| 315 | 1405.71 | 20.16 |
| 316 | 2587.16 | 21.07 |
| 317 | 5213.25 | 22.47 |
| 318 | 2320.16 | 20.73 |
| 319 | 4491.89 | 26.23 |
| 320 | 10199.91 | 21.11 |
| 321 | 854.38 | 34.92 |
| 322 | 1084.56 | 36.85 |
| 323 | 1814.78 | 37.29 |
| 324 | 2078.05 | 22.47 |
| 325 | 2175.08 | 33.26 |
| 326 | 2411.78 | 26.97 |
| 327 | 3738.59 | 24.76 |
| 328 | 3935.57 | 34.15 |
| 329 | 4863.21 | 26.66 |
| 330 | 860.39 | 26.25 |
| 331 | 1567.78 | 20.23 |
| 332 | 2308.11 | 27.32 |
| 333 | 2923.77 | 36.44 |
| 334 | 3295.55 | 25.40 |
| 335 | 3870.85 | 33.39 |
| 336 | 1099.56 | 21.63 |
| 337 | 1359.70 | 22.92 |
| 338 | 2059.02 | 23.12 |
| 339 | 2077.03 | 21.78 |
| 340 | 3349.34 | 35.81 |
| 341 | 8853.85 | 21.08 |
| 342 | 1734.80 | 20.24 |
| 343 | 1847.95 | 43.93 |
| 344 | 2045.95 | 34.04 |
| 345 | 2289.47 | 33.56 |
| 346 | 2421.15 | 34.74 |
| 347 | 2480.67 | 23.00 |
| 348 | 2576.25 | 34.17 |
| 349 | 3353.93 | 23.53 |
| 350 | 1083.52 | 26.24 |
| 351 | 2073.17 | 27.43 |
| 352 | 7958.65 | 34.32 |
| 353 | 1837.88 | 30.54 |
| 354 | 2939.03 | 33.75 |
| 355 | 2977.31 | 19.59 |
| 356 | 3596.46 | 21.54 |
| 357 | 3851.68 | 24.97 |
| 358 | 1135.52 | 27.83 |
| 359 | 3378.05 | 38.81 |
| 360 | 3590.72 | 28.99 |
| 361 | 3959.80 | 19.95 |
| 362 | 1258.41 | 36.10 |
| 363 | 1513.50 | 36.82 |
| 364 | 1716.38 | 20.59 |
| 365 | 2022.97 | 33.38 |
| 366 | 2914.54 | 24.29 |
| 367 | 5527.56 | 27.58 |
| 368 | 931.51 | 20.00 |
| 369 | 973.26 | 35.59 |
| 370 | 1385.67 | 27.92 |
| 371 | 2272.31 | 23.80 |
| 372 | 4024.87 | 33.20 |
| 373 | 2216.11 | 33.79 |
| 374 | 2756.23 | 35.16 |
| 375 | 2777.71 | 21.55 |
| 376 | 3521.02 | 30.73 |
| 377 | 3750.72 | 32.45 |
| 378 | 4229.09 | 29.08 |
| 379 | 4846.50 | 26.40 |
| 380 | 1046.55 | 25.35 |
| 381 | 1608.80 | 30.94 |
| 382 | 1878.78 | 31.58 |
| 383 | 2589.16 | 22.45 |
| 384 | 4369.06 | 20.25 |
| 385 | 12717.08 | 26.92 |
| 386 | 1210.43 | 36.48 |
| 387 | 3092.54 | 36.22 |
| 388 | 3248.61 | 25.65 |
| 389 | 4012.41 | 20.81 |
| 390 | 11016.34 | 21.31 |
| 391 | 1284.61 | 29.17 |
| 392 | 1460.83 | 22.53 |
| 393 | 1807.88 | 23.98 |
| 394 | 2596.33 | 34.86 |
| 395 | 2686.97 | 29.06 |
| 396 | 3871.59 | 27.51 |
| 397 | 4069.63 | 25.30 |
| 398 | 4288.98 | 25.94 |
| 399 | 4426.21 | 20.09 |
| 400 | 1071.55 | 21.41 |
| 401 | 1749.88 | 30.54 |
| 402 | 1956.97 | 21.44 |
| 403 | 2189.12 | 26.54 |
| 404 | 2257.63 | 36.10 |
| 405 | 2917.54 | 28.99 |
| 406 | 3633.69 | 26.99 |
| 407 | 6055.77 | 21.03 |
| 408 | 6186.02 | 24.99 |
| 409 | 1858.92 | 24.17 |
| 410 | 2274.11 | 33.47 |
| 411 | 4522.51 | 26.20 |
| 412 | 6237.35 | 31.39 |
| 413 | 9883.82 | 20.84 |
| 414 | 3385.6 | 25.47 |
| 415 | 3745.6 | 26.65 |
| 416 | 1408.7 | 39.13 |
| 417 | 2551.3 | 34.75 |
| 418 | 3265.3 | 36.02 |
| 419 | 2739.3 | 28.4 |
| 420 | 2065 | 24.48 |
| 421 | 2264.1 | 22.67 |
| 422 | 1058.5 | 24.94 |
| 423 | 4467.9 | 29.05 |
| 424 | 2887.4 | 35.66 |
| 425 | 1635.8 | 40.33 |
| 426 | 2525.2 | 27.72 |
| 427 | 1526.8 | 23.63 |
| 428 | 1664.8 | 29.87 |
| 429 | 2583.3 | 28.31 |
| 430 | 2663.3 | 23.44 |
| 431 | 1878.9 | 42.18 |
| 432 | 1462.7 | 39.31 |
| 433 | 1834.9 | 24 |
| 434 | 1893.1 | 24.64 |
| 435 | 1934 | 21.63 |
| 436 | 1367.7 | 38.87 |
| 437 | 1009.5 | 27.33 |
| 438 | 3405.1 | 25.92 |
| 439 | 2314.1 | 33.67 |
| 440 | 3996.8 | 20.93 |
| 441 | 2823.6 | 29.07 |
| 442 | 1179.6 | 27.15 |
| 443 | 1435.7 | 28.86 |
| 444 | 2430.7 | 35.39 |
| 445 | 1134.6 | 23.68 |
| 446 | 2014 | 25.18 |
| 447 | 2577.3 | 24.55 |
| 448 | 1194.6 | 26.73 |
| 449 | 1588.8 | 30.2 |
| 450 | 2056 | 25.44 |
| 451 | 2442.16 | 34.11 |
| 452 | 1422.66 | 21.72 |
| 453 | 1623.8 | 24.15 |
| 454 | 1624.61 | 37.73 |
| 455 | 3298.48 | 36.06 |
| 456 | 1016.31 | 35.67 |
| 457 | 1580.94 | 24.31 |
| 458 | 1157.58 | 37.41 |
| 459 | 1250.61 | 27.94 |
| 460 | 1378.67 | 28.85 |
| 461 | 1392.68 | 21.75 |
| 462 | 1409.64 | 22.06 |
| 463 | 1425.65 | 22.34 |
| 464 | 1451.71 | 29.19 |
| 465 | 1576.66 | 26.5 |
| 466 | 1651.85 | 40.6 |
| 467 | 1876.94 | 22.29 |
| 468 | 1911.12 | 24.98 |
| 469 | 2064.01 | 21.95 |
| 470 | 2150.04 | 27.76 |
| 471 | 2751.59 | 29.16 |
| 472 | 4289.94 | 28.69 |
| 473 | 4306.05 | 28.78 |
| 474 | 4800.18 | 23.83 |
| 475 | 1111.32 | 35.47 |
| 476 | 1181.49 | 36.79 |
| 477 | 3168.38 | 24.69 |
| 478 | 1229.57 | 36.29 |
| 479 | 1579.78 | 29.83 |
| 480 | 1680.82 | 30.02 |
| 481 | 1725.66 | 38.3 |
| 482 | 5228.15 | 27.04 |
| 483 | 1769.78 | 28.25 |
| 484 | 1114.54 | 25.52 |
| 485 | 1390.5 | 37.05 |
| 486 | 2046.99 | 32.56 |
| 487 | 2899.33 | 49.62 |
| 488 | 1096.53 | 26.12 |
| 489 | 1257.49 | 34.26 |
| 490 | 868.45 | 23.35 |
| 491 | 1160.43 | 35.6 |
| 492 | 1539.8 | 40.36 |
| 493 | 3318.91 | 36.01 |
| 494 | 1084.48 | 25.31 |
| 495 | 1388.39 | 58.99 |
| 496 | 3129.86 | 35.93 |
| 497 | 1255.56 | 36.33 |
| 498 | 1383.69 | 39.02 |
| 499 | 1561.75 | 40.72 |
| 500 | 3108.55 | 31.25 |
| 501 | 1173.58 | 37.51 |
| 502 | 1100.55 | 36.99 |
| 503 | 1128.44 | 33.71 |
| 504 | 3149.6 | 31.22 |
| 505 | 1068.56 | 21.69 |
| 506 | 1349.48 | 36.47 |
| 507 | 1689.81 | 40.57 |
| 508 | 2305.7 | 34.8 |
| 509 | 840.44 | 23.94 |
| 510 | 911.3 | 34.39 |
| 511 | 1299.64 | 22.42 |
| 512 | 911.47 | 25.92 |
| 513 | 1025.51 | 25.44 |
| 514 | 3400.07 | 42.03 |
| 515 | 1901.89 | 43.92 |
| 516 | 1110.42 | 34.37 |
| 517 | 1032.5 | 25.89 |
| 518 | 1040.52 | 25.11 |
| 519 | 1265.64 | 27.14 |
| 520 | 1171.55 | 29.24 |
| 521 | 1012.53 | 35.08 |
| 522 | 1286.49 | 36.78 |
| 523 | 2932.36 | 34.11 |
| 524 | 1215.49 | 27.61 |
| 525 | 1423.68 | 21.47 |
| 526 | 1487.71 | 29.58, |

wherein said CE times are based on capillary electrophoresis using a glass capillary of 90 cm in length with an inner diameter (ID) of 50 μm at an applied voltage of 30 kV,
wherein 20% acetonitrile and 0.25% formic acid in water is used as the mobile phase for the capillary electrophoresis at a flow rate of 2 μl/min., and
wherein said CE times are calibrated relative to the following values:

| Protein/Polypeptide | Migration time (minutes) |
|---|---|
| Aprotinin | 9.2 |
| Ribonuclease | 10.9 |
| Lysozyme | 8.9 |
| “REV” SEQ ID NO: 1 | 15.6 |
| “ELM” SEQ ID NO: 2 | 23.4 |
| “KINCON” SEQ ID NO: 3 | 20.0 |
| “GIVLY” SEQ ID NO: 4 | 36.8; |

determining if said subset of polypeptide markers comprises at least a first, second and third biomarker selected from:
a first biomarker that is at least one selected from the group consisting of:
(1) markers 1 to 106 (frequency markers) having (a) a frequency of presence in patients with VD of at least 0.4 and (b) a frequency of presence in patients with VD at least three (3) times the frequency of presence in patients without VD; (2) markers 1 to 106 (frequency markers) having (a) a frequency of presence in patients without VD of at least 0.4 and (b) a frequency of presence in patients without VD at least three (3) times the frequency of presence in patients with VD; (3) markers 107 to 526 (amplitude markers) having a mean amplitude in patients with VD of at least 200 and a mean amplitude at least two (2) times greater than the mean amplitude of the same marker in patients without VD; and (4) markers 107 to 526 (amplitude markers) having a mean amplitude in patients without VD of at least 200 and a mean amplitude at least two (2) times greater than the mean amplitude of the same marker in patients with VD;
a second biomarker which is different than said first biomarker and is at least one selected from the group consisting of: (1) markers 1 to 106 (frequency markers) having (a) a frequency of presence in patients with VD of at least 0.4 and (b) a frequency of presence in patients with VD at least three (3) times the frequency of presence in patients without VD; (2) markers 1 to 106 (frequency markers) having (a) a frequency of presence in patients without VD of at least 0.4 and (b) a frequency of presence in patients without VD at least three (3) times the frequency of presence in patients with VD; (3) markers 107 to 526 (amplitude markers) having a mean amplitude in patients with VD of at least 200 and a mean amplitude at least two (2) times greater than the mean amplitude of the same marker in patients without VD; and (4) markers 107 to 526 (amplitude markers) having a mean amplitude in patients without VD of at least 200 and a mean amplitude at least two (2) times greater than the mean amplitude of the same marker in patients with VD; and
a third biomarker which is different than said first and second biomarkers and is at least one selected from the group consisting of markers 1 to 526 (frequency and amplitude markers);
comparing the frequency and/or amplitude of presence of said at least first, second and third biomarkers in said sample of said subject patient to the frequency and/or amplitude of presence of the same at least first, second and third biomarkers from said known polypeptide markers from said control subjects,
ranking said subject patient between said control subjects with VD and without VD based on the second comparing step; and
diagnosing the probability of VD in said subject patient based on said ranking.

14. A method of diagnosing vascular diseases (VD) in a subject patient, comprising:
obtaining a urine sample from said subject;
pretreating said urine sample by a method selected from the group consisting of purification, separation, dilution, concentration or any combinations of the foregoing;
purifying said pretreated urine sample to remove high concentration proteins;
separating said pretreated and purified urine sample into a plurality of polypeptides using a combination of mass spectrometry (MS) and capillary electrophoresis (CE);
identifying said plurality of pretreated and purified polypeptides based on the separation characteristics of each of said plurality of pretreated and purified polypeptides in the separating step, wherein said separation characteristics comprise molecular mass and elution time;
comparing said plurality of pretreated and purified polypeptides to known polypeptide markers taken from control subjects with VD and without VD to obtain a subset of polypeptide markers from said plurality of pretreated and purified polypeptides that substantially match said known polypeptide markers, wherein said known polypeptide markers are characterized by the following molecular masses and migration times (CE time):

| No. | Mass | CE time |
|---|---|---|
| 1 | 1166.61 | 23.88 |
| 2 | 2431.50 | 24.10 |
| 3 | 1922.93 | 31.99 |
| 4 | 2509.16 | 25.76 |
| 5 | 3194.22 | 30.34 |
| 6 | 1705.80 | 40.47 |
| 7 | 1962.95 | 31.77 |
| 8 | 3822.12 | 24.72 |
| 9 | 2212.32 | 24.94 |
| 10 | 3015.78 | 35.86 |
| 11 | 1784.95 | 20.94 |
| 12 | 1902.92 | 31.87 |
| 13 | 2329.15 | 27.17 |
| 14 | 2154.05 | 21.78 |
| 15 | 2166.03 | 27.89 |
| 16 | 2258.27 | 21.99 |
| 17 | 2573.84 | 20.49 |
| 18 | 1270.75 | 37.92 |
| 19 | 1611.84 | 40.12 |
| 20 | 1791.87 | 41.04 |
| 21 | 2030.00 | 25.23 |
| 22 | 1290.40 | 30.87 |
| 23 | 1441.74 | 39.13 |
| 24 | 2924.25 | 24.05 |
| 25 | 816.41 | 21.10 |
| 26 | 963.52 | 21.71 |
| 27 | 1503.74 | 29.63 |
| 28 | 2849.59 | 23.02 |
| 29 | 3133.20 | 31.20 |
| 30 | 1283.62 | 27.30 |
| 31 | 1495.75 | 23.31 |
| 32 | 1513.70 | 29.29 |
| 33 | 1612.83 | 23.36 |
| 34 | 2319.19 | 33.80 |
| 35 | 2436.23 | 22.87 |
| 36 | 2557.42 | 28.22 |
| 37 | 2626.85 | 28.00 |
| 38 | 2933.46 | 27.68 |
| 39 | 2994.09 | 29.50 |
| 40 | 4101.34 | 28.51 |
| 41 | 935.49 | 23.69 |
| 42 | 1521.75 | 30.42 |
| 43 | 1669.79 | 21.48 |
| 44 | 2758.37 | 28.94 |
| 45 | 3546.94 | 26.22 |
| 46 | 3609.63 | 20.22 |
| 47 | 3697.49 | 23.71 |
| 48 | 4278.73 | 23.34 |
| 49 | 4421.04 | 20.73 |
| 50 | 4805.67 | 26.49 |
| 51 | 11058.18 | 21.88 |
| 52 | 1352.61 | 29.86 |
| 53 | 2802.85 | 36.35 |
| 54 | 4890.88 | 26.48 |
| 55 | 5212.06 | 26.98 |
| 56 | 945.45 | 25.80 |
| 57 | 1065.55 | 25.50 |
| 58 | 1137.58 | 26.41 |
| 59 | 1542.77 | 23.91 |
| 60 | 1693.83 | 23.47 |
| 61 | 3361.42 | 24.26 |
| 62 | 3617.74 | 26.97 |
| 63 | 3737.69 | 37.15 |
| 64 | 980.54 | 22.44 |
| 65 | 1221.63 | 26.82 |
| 66 | 2952.27 | 25.14 |
| 67 | 3696.88 | 26.94 |
| 38 | 5574.45 | 23.24 |
| 69 | 1182.59 | 28.34 |
| 70 | 1963.96 | 31.76 |
| 71 | 882.54 | 23.81 |
| 72 | 4002.72 | 20.69 |
| 73 | 4059.96 | 20.44 |
| 74 | 1186.59 | 22.31 |
| 75 | 1825.87 | 31.80 |
| 76 | 3401.66 | 23.42 |
| 77 | 1496.75 | 30.36 |
| 78 | 1832.92 | 31.91 |
| 79 | 2281.35 | 36.34 |
| 80 | 2344.34 | 33.66 |
| 81 | 3944.82 | 24.59 |
| 82 | 3002.23 | 23.80 |
| 83 | 3416.77 | 36.76 |
| 84 | 3501.86 | 31.79 |
| 85 | 6783.03 | 26.61 |
| 86 | 14111.27 | 21.97 |
| 87 | 2616.02 | 28.35 |
| 88 | 2810.45 | 36.73 |
| 89 | 2940.95 | 29.07 |
| 90 | 2946.45 | 34.96 |
| 91 | 1494.72 | 30.40 |
| 92 | 1080.53 | 27.86 |
| 93 | 2349.14 | 27.36 |
| 94 | 3303.00 | 23.07 |
| 95 | 4081.56 | 24.51 |
| 96 | 4670.27 | 25.84 |
| 37 | 4671.99 | 23.33 |
| 98 | 8933.94 | 22.57 |
| 99 | 1523.90 | 29.72 |
| 100 | 3956.82 | 25.20 |
| 101 | 3984.81 | 21.29 |
| 102 | 4830.78 | 26.61 |
| 103 | 3031.39 | 35.93 |
| 104 | 3788.76 | 25.21 |
| 105 | 2567.2 | 34.76 |
| 106 | 1447.8 | 19.49 |
| 107 | 2241.51 | 24.11 |
| 108 | 2461.11 | 30.84 |
| 109 | 1965.96 | 23.62 |
| 110 | 2189.08 | 27.17 |
| 111 | 1127.58 | 20.82 |
| 112 | 1400.71 | 20.35 |
| 113 | 1512.75 | 39.51 |
| 114 | 1860.53 | 34.24 |
| 115 | 1442.69 | 27.72 |
| 116 | 2590.78 | 27.96 |
| 117 | 1556.72 | 27.90 |
| 118 | 2309.15 | 21.95 |
| 119 | 2389.33 | 22.34 |
| 120 | 1478.68 | 39.28 |
| 121 | 1795.90 | 24.66 |
| 122 | 2211.03 | 35.06 |
| 123 | 1223.63 | 19.52 |
| 124 | 1829.04 | 21.22 |
| 125 | 1878.66 | 30.19 |
| 126 | 2009.96 | 32.27 |
| 127 | 2110.00 | 24.10 |
| 128 | 1552.79 | 29.75 |
| 129 | 1577.75 | 40.03 |
| 130 | 1936.94 | 34.71 |
| 131 | 2368.13 | 26.75 |
| 132 | 3633.05 | 33.25 |
| 133 | 1510.72 | 28.30 |
| 134 | 1668.87 | 40.49 |
| 135 | 2227.05 | 33.43 |
| 136 | 1495.75 | 39.41 |
| 137 | 1631.77 | 45.38 |
| 138 | 3158.60 | 29.62 |
| 139 | 1522.78 | 22.76 |
| 140 | 1727.87 | 39.61 |
| 141 | 1883.94 | 40.14 |
| 142 | 1460.71 | 19.83 |
| 143 | 1805.88 | 29.92 |
| 144 | 1898.93 | 40.30 |
| 145 | 2237.06 | 27.12 |
| 146 | 3178.33 | 30.26 |
| 147 | 1844.56 | 34.28 |
| 148 | 1378.57 | 37.16 |
| 149 | 1764.86 | 29.88 |
| 150 | 1791.88 | 30.77 |
| 151 | 2082.01 | 33.67 |
| 152 | 1768.90 | 20.77 |
| 153 | 3442.09 | 33.32 |
| 154 | 876.42 | 35.07 |
| 155 | 2352.14 | 26.74 |
| 156 | 937.50 | 34.12 |
| 157 | 1445.72 | 28.36 |
| 158 | 1893.10 | 28.85 |
| 159 | 2839.43 | 24.14 |
| 160 | 1600.76 | 29.61 |
| 161 | 1565.75 | 26.35 |
| 162 | 1627.76 | 29.47 |
| 163 | 1812.90 | 39.98 |
| 164 | 3137.52 | 30.29 |
| 165 | 1364.67 | 28.65 |
| 166 | 2298.07 | 33.82 |
| 167 | 3017.74 | 49.66 |
| 168 | 1235.61 | 26.67 |
| 169 | 1741.81 | 30.21 |
| 170 | 1818.90 | 30.93 |
| 171 | 1892.95 | 22.22 |
| 172 | 3280.69 | 22.69 |
| 173 | 2658.34 | 19.50 |
| 174 | 1407.71 | 27.46 |
| 175 | 1622.79 | 26.82 |
| 176 | 1684.78 | 29.64 |
| 177 | 1321.65 | 28.39 |
| 178 | 1350.68 | 27.13 |
| 179 | 1549.76 | 39.52 |
| 180 | 2233.10 | 22.47 |
| 181 | 2679.27 | 23.48 |
| 182 | 1835.79 | 20.02 |
| 183 | 3421.66 | 25.96 |
| 184 | 1708.85 | 30.44 |
| 185 | 1993.96 | 32.16 |
| 186 | 2695.31 | 23.46 |
| 187 | 1204.65 | 21.93 |
| 188 | 1467.86 | 24.38 |
| 189 | 1767.07 | 24.10 |
| 190 | 8176.30 | 19.57 |
| 191 | 1143.56 | 36.97 |
| 192 | 1834.90 | 31.05 |
| 193 | 2025.95 | 32.21 |
| 194 | 3489.70 | 31.45 |
| 195 | 1268.62 | 27.29 |
| 196 | 1659.82 | 29.35 |
| 197 | 2405.59 | 22.16 |
| 198 | 2483.21 | 27.54 |
| 199 | 2599.21 | 28.20 |
| 200 | 4170.01 | 33.51 |
| 201 | 1013.41 | 25.33 |
| 202 | 1016.49 | 25.88 |
| 203 | 1493.74 | 22.06 |
| 204 | 2104.04 | 32.97 |
| 205 | 3718.81 | 32.39 |
| 206 | 4251.98 | 28.66 |
| 207 | 4538.67 | 26.20 |
| 208 | 2067.93 | 20.68 |
| 209 | 2292.11 | 27.26 |
| 210 | 3702.39 | 32.39 |
| 211 | 965.46 | 27.84 |
| 212 | 2186.07 | 25.89 |
| 213 | 2584.29 | 35.08 |
| 214 | 2841.13 | 24.50 |
| 215 | 9866.78 | 20.85 |
| 216 | 1099.53 | 28.33 |
| 217 | 2471.25 | 34.69 |
| 218 | 3734.85 | 32.41 |
| 219 | 3927.86 | 33.50 |
| 220 | 3166.32 | 22.10 |
| 221 | 2339.08 | 33.95 |
| 222 | 2563.76 | 22.05 |
| 223 | 3219.35 | 35.00 |
| 224 | 3359.66 | 31.84 |
| 225 | 4097.98 | 24.59 |
| 226 | 4654.14 | 25.81 |
| 227 | 1584.77 | 29.72 |
| 228 | 2148.10 | 25.54 |
| 229 | 2639.45 | 21.33 |
| 230 | 3013.27 | 22.27 |
| 231 | 3205.39 | 19.71 |
| 232 | 3831.86 | 28.39 |
| 233 | 1050.52 | 27.03 |
| 234 | 2157.06 | 22.19 |
| 235 | 2407.16 | 27.65 |
| 236 | 2837.93 | 23.99 |
| 237 | 3058.02 | 30.20 |
| 238 | 1658.67 | 21.53 |
| 239 | 3311.32 | 24.46 |
| 240 | 3556.63 | 23.64 |
| 241 | 1085.50 | 21.70 |
| 242 | 1199.63 | 21.91 |
| 243 | 1247.58 | 22.00 |
| 244 | 1608.76 | 22.36 |
| 245 | 2501.20 | 34.30 |
| 246 | 3021.52 | 23.52 |
| 247 | 4153.75 | 33.41 |
| 248 | 5000.17 | 24.43 |
| 249 | 8917.48 | 22.53 |
| 250 | 1750.86 | 23.80 |
| 251 | 2235.13 | 34.10 |
| 252 | 2644.25 | 21.13 |
| 253 | 3943.96 | 33.53 |
| 254 | 11967.96 | 20.50 |
| 255 | 2553.23 | 34.14 |
| 256 | 1209.58 | 26.31 |
| 257 | 1899.94 | 21.41 |
| 258 | 1680.00 | 23.77 |
| 259 | 2195.06 | 20.15 |
| 260 | 3064.41 | 20.55 |
| 261 | 3554.07 | 31.11 |
| 262 | 3686.03 | 22.16 |
| 263 | 3802.12 | 33.10 |
| 264 | 4048.05 | 25.42 |
| 265 | 2380.16 | 36.48 |
| 266 | 1352.83 | 24.38 |
| 267 | 1638.80 | 20.26 |
| 268 | 2864.18 | 20.19 |
| 269 | 3754.66 | 37.16 |
| 270 | 4185.91 | 33.47 |
| 271 | 858.42 | 23.26 |
| 272 | 1159.64 | 26.05 |
| 273 | 1407.71 | 37.25 |
| 274 | 1439.72 | 29.62 |
| 275 | 1720.76 | 19.72 |
| 276 | 1846.93 | 32.04 |
| 277 | 3177.14 | 22.48 |
| 278 | 4113.80 | 24.58 |
| 279 | 2744.07 | 35.03 |
| 280 | 2767.26 | 21.52 |
| 281 | 1310.64 | 27.11 |
| 282 | 1613.88 | 23.95 |
| 283 | 1703.90 | 33.64 |
| 284 | 2761.40 | 21.46 |
| 285 | 3242.42 | 22.78 |
| 286 | 3338.17 | 23.36 |
| 287 | 3371.74 | 22.96 |
| 288 | 3593.53 | 20.25 |
| 289 | 3677.52 | 24.49 |
| 290 | 1624.80 | 30.81 |
| 291 | 2210.92 | 37.55 |
| 292 | 3290.37 | 24.12 |
| 293 | 4413.76 | 29.03 |
| 294 | 1482.73 | 22.47 |
| 295 | 1813.78 | 31.87 |
| 296 | 1934.87 | 20.04 |
| 297 | 2249.89 | 34.14 |
| 298 | 3280.59 | 25.76 |
| 299 | 1098.56 | 21.46 |
| 300 | 1125.58 | 21.76 |
| 301 | 1649.79 | 19.59 |
| 302 | 4025.68 | 20.73 |
| 303 | 980.33 | 35.59 |
| 304 | 1096.41 | 35.95 |
| 305 | 1698.65 | 37.60 |
| 306 | 2361.21 | 20.77 |
| 307 | 3148.50 | 24.22 |
| 308 | 3157.23 | 34.74 |
| 309 | 1304.59 | 27.95 |
| 310 | 3575.78 | 32.27 |
| 311 | 1510.75 | 20.12 |
| 312 | 2485.20 | 34.25 |
| 313 | 3076.33 | 19.64 |
| 314 | 3343.39 | 31.80 |
| 315 | 1405.71 | 20.16 |
| 316 | 2587.16 | 21.07 |
| 317 | 5213.25 | 22.47 |
| 318 | 2320.16 | 20.73 |
| 319 | 4491.89 | 26.23 |
| 320 | 10199.91 | 21.11 |
| 321 | 854.38 | 34.92 |
| 322 | 1084.56 | 36.85 |
| 323 | 1814.78 | 37.29 |
| 324 | 2078.05 | 22.47 |
| 325 | 2175.08 | 33.26 |
| 326 | 2411.78 | 26.97 |
| 327 | 3738.59 | 24.76 |
| 328 | 3935.57 | 34.15 |
| 329 | 4863.21 | 26.66 |
| 330 | 860.39 | 26.25 |
| 331 | 1567.78 | +0.23 |
| 332 | 2308.11 | 27.32 |
| 333 | 2923.77 | 36.44 |
| 334 | 3295.55 | 25.40 |
| 335 | 3870.85 | 33.39 |
| 336 | 1099.56 | 21.63 |
| 337 | 1359.70 | 22.92 |
| 338 | 2059.02 | 23.12 |
| 339 | 2077.03 | 21.78 |
| 340 | 3349.34 | 35.81 |
| 341 | 8853.85 | 21.08 |
| 342 | 1734.80 | 20.24 |
| 343 | 1847.95 | 43.93 |
| 344 | 2045.95 | 34.04 |
| 345 | 2289.47 | 33.56 |
| 346 | 2421.15 | 34.74 |
| 347 | 2480.67 | 23.00 |
| 348 | 2576.25 | 34.17 |
| 349 | 3353.93 | 23.53 |
| 350 | 1083.52 | 26.24 |
| 351 | 2073.17 | 27.43 |
| 352 | 7958.65 | 34.32 |
| 353 | 1837.88 | 30.54 |
| 354 | 2939.03 | 33.75 |
| 355 | 2977.31 | 19.59 |
| 356 | 3596.46 | 21.54 |
| 357 | 3851.68 | 24.97 |
| 358 | 1135.52 | 27.83 |
| 359 | 3378.05 | 38.81 |
| 360 | 3590.72 | 28.99 |
| 361 | 3959.80 | 19.95 |
| 362 | 1258.41 | 36.10 |
| 363 | 1513.50 | 36.82 |
| 364 | 1716.38 | 20.59 |
| 365 | 2022.97 | 33.38 |
| 366 | 2914.54 | 24.29 |
| 367 | 5527.56 | 27.58 |
| 368 | 931.51 | 20.00 |
| 369 | 973.26 | 35.59 |
| 370 | 1385.67 | 27.92 |
| 371 | 2272.31 | 23.80 |
| 372 | 4024.87 | 33.20 |
| 373 | 2216.11 | 33.79 |
| 374 | 2756.23 | 35.16 |
| 375 | 2777.71 | 21.55 |
| 376 | 3521.02 | 30.73 |
| 377 | 3750.72 | 32.45 |
| 378 | 4229.09 | 29.08 |
| 379 | 4846.50 | 26.40 |
| 380 | 1046.55 | 25.35 |
| 381 | 1608.80 | 30.94 |
| 382 | 1878.78 | 31.58 |
| 383 | 2589.16 | 22.45 |
| 384 | 4369.06 | 20.25 |
| 385 | 12717.08 | 26.92 |
| 386 | 1210.43 | 36.48 |
| 387 | 3092.54 | 36.22 |
| 388 | 3248.61 | 25.65 |
| 389 | 4012.41 | 20.81 |
| 390 | 11016.34 | 21.31 |
| 391 | 1284.61 | 29.17 |
| 392 | 1460.83 | 22.53 |
| 393 | 1807.88 | 23.98 |
| 394 | 2596.33 | 34.86 |
| 395 | 2686.97 | 29.06 |
| 396 | 3871.59 | 27.51 |
| 397 | 4069.63 | 25.30 |
| 398 | 4288.98 | 25.94 |
| 399 | 4426.21 | 20.09 |
| 400 | 1071.55 | 21.41 |
| 401 | 1749.88 | 30.54 |
| 402 | 1956.97 | 21.44 |
| 403 | 2189.12 | 26.54 |
| 404 | 2257.63 | 36.10 |
| 405 | 2917.54 | 28.99 |
| 406 | 3633.69 | 26.99 |
| 407 | 6055.77 | 21.03 |
| 408 | 6186.02 | 24.99 |
| 409 | 1858.92 | 24.17 |
| 410 | 2274.11 | 33.47 |
| 411 | 4522.51 | 26.20 |
| 412 | 6237.35 | 31.39 |
| 413 | 9883.82 | 20.84 |
| 414 | 3385.6 | 25.47 |
| 415 | 3745.6 | 26.65 |
| 416 | 1408.7 | 39.13 |
| 417 | 2551.3 | 34.75 |
| 418 | 3265.3 | 36.02 |
| 419 | 2739.3 | 28.4 |
| 420 | 2065 | 24.48 |
| 421 | 2264.1 | 22.67 |
| 422 | 1058.5 | 24.94 |
| 423 | 4467.9 | 29.05 |
| 424 | 2887.4 | 35.66 |
| 425 | 1635.8 | 40.33 |
| 426 | 2525.2 | 27.72 |
| 427 | 1526.8 | 23.63 |
| 428 | 1664.8 | 29.87 |
| 429 | 2583.3 | 28.31 |
| 430 | 2663.3 | 23.44 |
| 431 | 1878.9 | 42.18 |
| 432 | 1462.7 | 39.31 |
| 433 | 1834.9 | 24 |
| 434 | 1893.1 | 24.64 |
| 435 | 1934 | 21.63 |
| 436 | 1367.7 | 38.87 |
| 437 | 1009.5 | 27.33 |
| 438 | 3405.1 | 25.92 |
| 439 | 2314.1 | 33.67 |
| 440 | 3996.8 | 20.93 |
| 441 | 2823.6 | 29.07 |
| 442 | 1179.6 | 27.15 |
| 443 | 1435.7 | 28.86 |
| 444 | 2430.7 | 35.39 |
| 445 | 1134.6 | 23.68 |
| 446 | 2014 | 25.18 |
| 447 | 2577.3 | 24.55 |
| 448 | 1194.6 | 26.73 |
| 449 | 1588.8 | 30.2 |
| 450 | 2056 | 25.44 |
| 451 | 2442.16 | 34.11 |
| 452 | 1422.66 | 21.72 |
| 453 | 1623.8 | 24.15 |
| 454 | 1624.61 | 37.73 |
| 455 | 3298.48 | 36.06 |
| 456 | 1016.31 | 35.67 |
| 457 | 1580.94 | 24.31 |
| 458 | 1157.58 | 37.41 |
| 459 | 1250.61 | 27.94 |
| 460 | 1378.67 | 28.85 |
| 461 | 1392.68 | 21.75 |
| 462 | 1409.64 | 22.06 |
| 463 | 1425.65 | 22.34 |
| 464 | 1451.71 | 29.19 |
| 465 | 1576.66 | 26.5 |
| 466 | 1651.85 | 40.6 |
| 467 | 1876.94 | 22.29 |
| 468 | 1911.12 | 24.98 |
| 469 | 2064.01 | 21.95 |
| 470 | 2150.04 | 27.76 |
| 471 | 2751.59 | 29.16 |
| 472 | 4289.94 | 28.69 |
| 473 | 4306.05 | 28.78 |
| 474 | 4800.18 | 23.83 |
| 475 | 1111.32 | 35.47 |
| 476 | 1181.49 | 36.79 |
| 477 | 3168.38 | 24.69 |
| 478 | 1229.57 | 36.29 |
| 479 | 1579.78 | 29.83 |
| 480 | 1680.82 | 30.02 |
| 481 | 1725.66 | 38.3 |
| 482 | 5228.15 | 27.04 |
| 483 | 1769.78 | 28.25 |
| 484 | 1114.54 | 25.52 |
| 485 | 1390.5 | 37.05 |
| 486 | 2046.99 | 32.56 |
| 487 | 2899.33 | 49.62 |
| 488 | 1096.53 | 26.12 |
| 489 | 1257.49 | 34.26 |
| 490 | 868.45 | 23.35 |
| 491 | 1160.43 | 35.6 |
| 492 | 1539.8 | 40.36 |
| 493 | 3318.91 | 36.01 |
| 494 | 1084.48 | 25.31 |
| 495 | 1388.39 | 58.99 |
| 496 | 3129.86 | 35.93 |
| 497 | 1255.56 | 36.33 |
| 498 | 1383.69 | 39.02 |
| 499 | 1561.75 | 40.72 |
| 500 | 3108.55 | 31.25 |
| 501 | 1173.58 | 37.51 |
| 502 | 1100.55 | 36.99 |
| 503 | 1128.44 | 33.71 |
| 504 | 3149.6 | 31.22 |
| 505 | 1068.56 | 21.69 |
| 506 | 1349.48 | 36.47 |
| 507 | 1689.81 | 40.57 |
| 508 | 2305.7 | 34.8 |
| 509 | 840.44 | 23.94 |
| 510 | 911.3 | 34.39 |
| 511 | 1299.64 | 22.42 |
| 512 | 911.47 | 25.92 |
| 513 | 1025.51 | 25.44 |
| 514 | 3400.07 | 42.03 |
| 515 | 1901.89 | 43.92 |
| 516 | 1110.42 | 34.37 |
| 517 | 1032.5 | 25.89 |
| 518 | 1040.52 | 25.11 |
| 519 | 1265.64 | 27.14 |
| 520 | 1171.55 | 29.24 |
| 521 | 1012.53 | 35.08 |
| 522 | 1286.49 | 36.78 |
| 523 | 2932.36 | 34.11 |
| 524 | 1215.49 | 27.61 |
| 525 | 1423.68 | 21.47 |
| 526 | 1487.71 | 29.58, |

wherein said CE times are based on capillary electrophoresis using a glass capillary of 90 cm in length with an inner diameter (ID) of 50 μm at an applied voltage of 30 kV,
wherein 20% acetonitrile and 0.25% formic acid in water is used as the mobile phase for the capillary electrophoresis at a flow rate of 2 μl/min., and
wherein said CE times are calibrated relative to the following values:

| Protein/Polypeptide | Migration time (minutes) |
|---|---|
| Aprotinin | 9.2 |
| Ribonuclease | 10.9 |
| Lysozyme | 8.9 |
| “REV” SEQ ID NO: 1 | 15.6 |
| “ELM” SEQ ID NO: 2 | 23.4 |
| “KINCON” SEQ ID NO: 3 | 20.0 |
| “GIVLY” SEQ ID NO: 4 | 36.8; |

determining if said subset of polypeptide markers comprises at least a first, second and third biomarker selected from:
a first biomarker that is at least one selected from the group consisting of:
(1) markers 1-6, 10-12, 15-16, 18, 20-21 and 23-24 (frequency markers); (2) markers 25-28, 31-32, 35, 37, 40, 42, 44-46, 48-51, 53-62, 64-66, 70-71, 73, 77-85, 87-90, 92-96 and 98-104 (frequency markers); (3) markers 107-108, 110, 112, 114-115, 118-123, 125-138, 140-142, 144-147, 149, 151, 154-158, 160-164, 166, 169-171, 173-176, 178-179, 181, 183, 185-186, 189, 191-197, 199, 201, 204, 212, 214, 227-228, 237, 256, 261, 274, 276, 281, 291, 295, 309, 319, 321, 325, 344, 382, 474-475, 477, 485, 487, 489, 494-495, 503, 512, 514, 521 and 524-525 (amplitude markers); and (4) markers 124, 139, 148, 152-153, 159, 167-168, 172, 177, 180, 182, 184, 187-188, 190, 198, 200, 202-203, 204-205, 207-210, 213, 215-222, 224-226, 229-236, 238, 240-244, 246-255, 258-260, 262-263, 266-268, 271-273, 277-278, 280, 282-289, 292-294, 296, 298, 300, 302, 305-306, 313-316, 320, 327, 329-334, 337-339, 341-343, 349, 352-356, 397, 399, 401, 404-406, 415-417, 422, 425, 433, 435, 443, 453 and 466 (amplitude markers);
a second biomarker which is different than said first biomarker and is at least one selected from the group consisting of: (1) markers 1-6, 10-12, 15-16, 18, 20-21 and 23-24 (frequency markers); (2) markers 25-28, 31-32, 35, 37, 40, 42, 44-46, 48-51, 53-62, 64-66, 70-71, 73, 77-85, 87-90, 92-96 and 98-104 (frequency markers); (3) markers 107-108, 110, 112, 114-115, 118-123, 125-138, 140-142, 144-147, 149, 151, 154-158, 160-164, 166, 169-171, 173-176, 178-179, 181, 183, 185-186, 189, 191-197, 199, 201, 204, 212, 214, 227-228, 237, 256, 261, 274, 276, 281, 291, 295, 309, 319, 321, 325, 344, 382, 474-475, 477, 485, 487, 489, 494-495, 503, 512, 514, 521 and 524-525 (amplitude markers); and (4) markers 124, 139, 148, 152-153, 159, 167-168, 172, 177, 180, 182, 184, 187-188, 190, 198, 200, 202-203, 204-205, 207-210, 213, 215-222, 224-226, 229-236, 238, 240-244, 246-255, 258-260, 262-263, 266-268, 271-273, 277-278, 280, 282-289, 292-294, 296, 298, 300, 302, 305-306, 313-316, 320, 327, 329-334, 337-339, 341-343, 349, 352-356, 397, 399, 401, 404-406, 415-417, 422, 425, 433, 435, 443, 453 and 466 amplitude makers; and
a third biomarker which is different than said first and second biomarkers and is at least one selected from the group consisting of markers 1 to 526 (frequency and amplitude markers);
comparing the frequency and/or amplitude of presence of said at least first, second and third biomarkers in said sample of said subject patient to the frequency and/or amplitude of presence of the same at least first, second and third biomarkers from said known polypeptide markers from said control subjects,
ranking said subject patient between said control subjects with VD and without VD based on the second comparing step; and
diagnosing the probability of VD in said subject patient based on said ranking.


SEQUENCE LISTING (4 sequences)

SEQ ID NO: 1 - protein, 15 aa, Artificial
  Synthetic peptide
REVQSKIGYGRQIIS

SEQ ID NO: 2 - protein, 24 aa, Artificial
  Synthetic peptide
ELMTGELPYSHINNRDQIIFMVGR

SEQ ID NO: 3 - protein, 21 aa, Artificial
  Synthetic peptide
TGSLPYSHIGSRDQIIFMVGR

SEQ ID NO: 4 - protein, 18 aa, Artificial
  Synthetic peptide
GIVLYELMTGELPYSHIN